(19) United States
(12) Patent Application Publication
(10) Pub. No.: US 2026/0272926 A1
(43) Pub. Date: Sep. 17, 2026

(54) HIV-1P ROTEASE INHIBITORS AND USES THEREOF

(71) Applicant: Purdue Research Foundation, West Lafayette, IN (US)

(72) Inventors: Arun K. Ghosh, West Lafayette, IN (US); Hiroaki Mitsuya, Kumamoto (JP)

(21) Appl. No.: 19/168,690

(22) PCT Filed: Mar. 22, 2024
(86) PCT No.: PCT/US2024/021035

§ 371 Date: Sep. 24, 2025

Related U.S. Application Data
(60) Provisional application No. 63/492,139, filed on Mar. 24, 2023.

(51) Int. Cl. A61K 31/506; A61K 31/426; A61K 31/4436; A61P 31/18; C07D 239/42; C07D 277/42; C07D 401/04; C07D 417/04; C07D 417/14

(52) U.S. Cl. CPC A61K 31/506; A61K 31/426; A61K 31/4436; A61P 31/18; C07D 239/42; C07D 277/42; C07D 401/04; C07D 417/04; C07D 417/14

(57) ABSTRACT

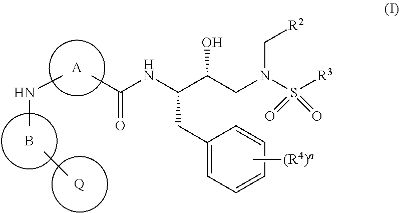

Compounds of the formula (I) or pharmaceutically acceptable salts thereof, wherein A is aryl; B is heterocyclyl; Q is alkyl or heterocyclyl; R² is alkyl; R³ is aryl, benzthiazole, benzoxazole, benzofuranyl or indolyl; each R⁴ is, independently, alkyl, alkoxy, aryl, heterocyclyl, halo, hydroxy or amino; and n is an integer from 0 to 3; methods for treating an HIV infection or AIDS comprising administering a therapeutically effective amount of one or more compounds of formula (I) to a subject in need thereof.

### CROSS-REFERENCE TO RELATED APPLICATIONS

This application claims the benefit of U.S. Provisional Appl. No. 63/492,139, filed Mar. 24, 2023.

### STATEMENT OF GOVERNMENT SUPPORT

This invention was made with government support under AI150466 awarded by the National Institutes of Health. The government has certain rights in the invention.

### BACKGROUND

Therapeutic inhibition of virally encoded HIV-1 protease is one of the most important strategies for the treatment of HIV-1 infection and AIDS. The development of HIV-1 protease inhibitor drugs using protein X-ray structure-based design is regarded as a major achievement in medicinal chemistry. Protease inhibitors have been an integral part of current antiretroviral therapies (cART) with reverse transcriptase inhibitors, which emerged in the mid-1990s. The CART treatment regimens have dramatically transformed HIV/AIDS into a manageable chronic ailment. As a result, the HIV-related mortality rates have continued to decline due to improved treatment regimens. Despite these major improvements, the growing emergence of drug-resistant strains, high pill burden and drug side effects are becoming major concerns regarding the long-term prospects of HIV/AIDS management. In particular, the first generation of HIV-1 protease inhibitors were associated with peptide-like features, which contributed to poor oral bioavailability, high metabolic degradation, and other debilitating side effects. The development of second-generation protease inhibitor drugs alleviated some of these major issues; however, current treatments are far from ideal due to the emergence of drug resistance and cardiovascular and CNS (central nervous system) problems. In our continuing efforts towards the development of improved treatment options, our protease inhibitor design strategy has been aimed at maximizing hydrogen bonding interactions with the active site protease backbone atoms. This strategy has resulted in a range of highly potent inhibitors, including the FDA-approved inhibitor drug darunavir and related compound TMC-126. Darunavir exhibits a dual mechanism of action in which it inhibits the catalytically active dimeric HIV-1 protease and also inhibits dimerization of the individual protease monomers, preventing the formation of catalytically active protease enzyme.

Darunavir utilizes a unique design strategy that involved promoting a network of hydrogen-bonding interactions with protease backbone S2 to S2′ sites of the active site. These properties most likely contributed to darunavir's high genetic barrier to the development of drug-resistant variants compared to other approved protease inhibitor drugs. While darunavir is used widely as a first line therapy for rescue treatment, darunavir-resistant HIV-1 variants have emerged. Therefore, further development of a new class of protease inhibitors with structurally novel features is important for future treatment options.

### SUMMARY

Making reference to Scheme 1, one of the important features of darunavir is the bicyclic polyether-like P2 ligand, the bis-tetrahydrofuranyl heterocycle. Both oxygens of this polyether scaffold form tight hydrogen bonds with backbone amide NHs of Asp29 and Asp30 in the S2 subsite. To design new functionalities that can accommodate similar hydrogen bonding interactions with the backbone atoms the pyridylpyrimidine scaffold of imatinib 3 or nilotinib 4 can be incorporated with substituted 3-aminobenzoic acid to mimic hydrogen bonding interactions in the S2 subsite similar to the bis-THF ligand of darunavir. Nilotinib is a selective tyrosine kinase receptor inhibitor used for the treatment of chronic myelogenous leukemia. Nilotinib binds to and stabilizes the inactive conformation of the kinase domain of the Abl protein of the Bcr-Abl fusion protein. This results in the inhibition of the Bcr-Abl-mediated proliferation of Philadelphia chromosome-positive (Ph+) chronic myeloid leukemia (CML) cells. Nilotinib is orally bioavailable, and incorporation of its structural features may improve pharmacological properties of the resulting protease inhibitors.

The X-ray structural studies of imatinib- and nilotinib-bound Abl-Bcr kinase revealed donor-acceptor abilities of pyridylpirimidine hetereocycles. The major advantage is that such heterocyclic ligands do not have stereochemical complexities related to bis-THF ligand. We describe here our effort towards the design of a new class of HIV-1 protease inhibitors by incorporating a variety of hinge-binding heterocycles that would maintain the critical hydrogen bonding interactions in the S2 subsite of HIV-1 protease. The structural class is represented in compound 5. These heterocyclic scaffolds are key templates for kinase inhibitor drugs, imatinib and dasatinib. Described in greater detail herein, new ligand scaffolds in combination with the hydroxyethylamine sulfonamide isostere inherent to darunavir and TMC-126 were developed.

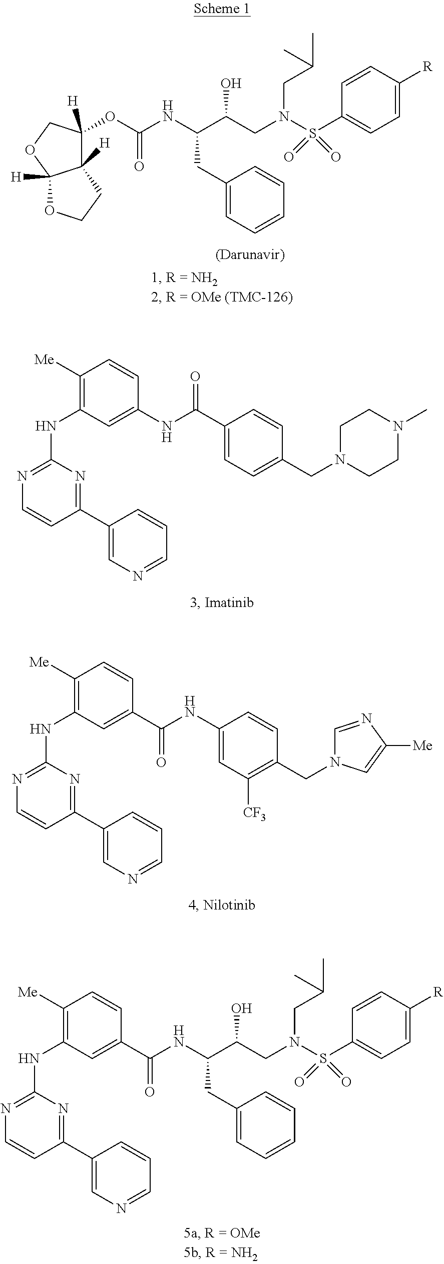

### DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

The disclosure relates to a compound of the formula (I):

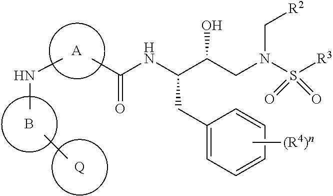

or a pharmaceutically acceptable salt thereof, wherein:
A is aryl;
B is heterocyclyl;
Q is alkyl or heterocyclyl;
R² is alkyl;
R³ is aryl, benzthiazole, benzoxazole, benzofuranyl or indolyl;
each R⁴ is, independently, alkyl, alkoxy, aryl, heterocyclyl, halo, hydroxy or amino; and
n is an integer from 0 to 3.

In one example, A is a monocyclic aryl group, such as a substituted monocyclic aryl group. Thus, for example, A can be an alkyl-substituted monocyclic aryl group. In one example, A can be phenyl.

In addition, or alternatively, to the foregoing, B can be a five- or six-membered monocyclic heterocyclyl group. For example, B can be a monocyclic heteroaryl group of the formula:

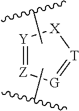

wherein X, Y, Z, G, and T are each, independently, C, CR⁵, N, NR⁶, S or O, wherein each R⁵ is independently H, alkyl or amino; and each R⁶ is independently H or alkyl. For example, X can be S and G can be N, such as when X is S, G is N, and Z is CR⁵, wherein each R⁵ can independently be H or (C₁-C₃)alkyl. In one example, each R⁵ is H. Thus, for example, B can be of the formula:

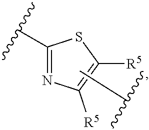

wherein each R⁵ can be H, it being understood that one R⁵ would be absent if the bond with the wavy line is attached that carbon atom. B can be, for example, of the formula:

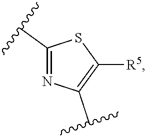

such that B can be attached to A and Q as shown:

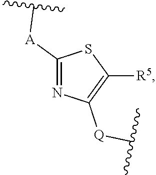

wherein R⁵ can be H.

Alternatively, B can be a six-membered monocyclic heterocyclyl group. The six-membered monocyclic heterocyclyl group B can be a monocyclic heteroaryl group of the formula:

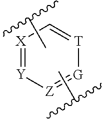

wherein X, Y, Z, G, and T are each, independently, C, CR⁵ or N, wherein each R⁵ is independently H, alkyl or amino. In one example, at least one of X, Y, Z, G, and T is N. In another example, at least two of X, Y, Z, G, and T is N. In examples wherein X, Y, Z, G, and T are CR⁵, R⁵ can be H or (C₁-C₃)alkyl. In examples wherein X, Y, Z, G, and T are CR⁵, R⁵ can be H.

The group B can be of the formula:

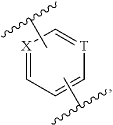

such as a group of the formula:

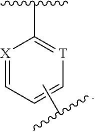

In one example, at least one of X and T is N. In another example X and T are N. Thus, for example B can be a group of the formula:

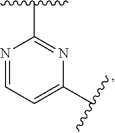 [SMILES: *c1ccnc(*)n1]

such that B can be attached to A and Q as shown:

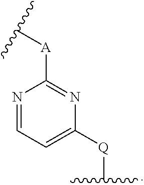

In addition, or alternatively, to the foregoing, Q can be a six-membered monocyclic heterocyclyl group, such as a six-membered monocyclic heterocyclyl group. Q can be a monocyclic heteroaryl group of the formula:

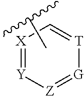

wherein X, Y, Z, G, and T are each, independently, C, CR⁵ or N, wherein each R⁵ is independently H, alkyl or amino. In one example, at least one of X, Y, Z, G, and T is N, such as wherein at least two of X, Y, Z, G, and T is N. R⁵ can be, for example, H or (C₁-C₃)alkyl. In some examples, R⁵ is H.

The group Q can be, for example, a group of the formula:

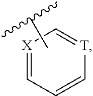

such as a group of the formula:

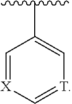

Thus, for example, at least one of X and T can be N; or X can be N and T can be CR⁵, such that the group Q is of the formula:

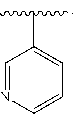 [SMILES: *c1cccnc1]

Alternatively, Q can be alkyl, such as (C₁-C₃)alkyl. Q can be substituted. Non-limiting examples of substituents that can be present on Q can be alkyl, halo, alkoxy, amino or aryl. Thus, for example, Q can be haloalkyl or alkoxyalkyl. Examples of haloalkyl include, mono-, di- and tri-fluoroalkyl. And examples of alkoxyalkyl include (C₁-C₃)alkyl-O—(C₁-C₃)alkyl, such as —CH₂OCH₃.

Examples of compounds of the formula (I) include the compounds of the formula (Ia)-(If) shown in Table A:

TABLE A

| | |
|---|---|
| 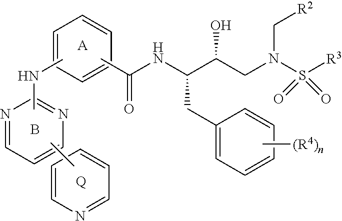 | (Ia) |
| 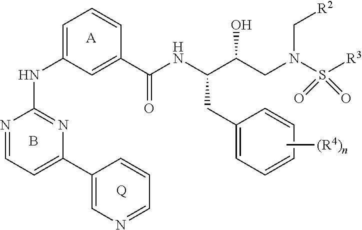 | (Id) |
| 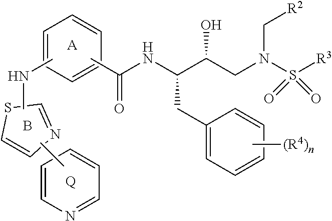 | (Ib) |
| 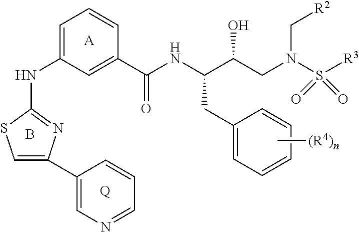 | (Ie) |
| 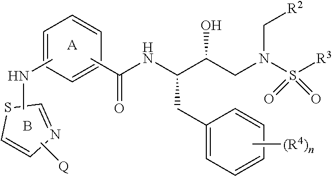 | (Ic) |
| wherein Q is alkyl as | |
| defined herein. | |
| 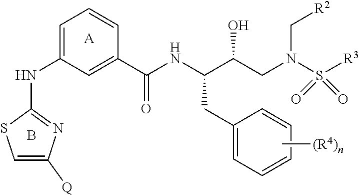 | (If) |
| wherein Q is alkyl as defined | |
| herein. | |

Although the groups A, B, and Q shown in Table A are shown without substituents, it should be understood that each group can be substituted.

In any of the compounds of the formulae (I) and (Ia)-(If), R³ can be aryl, such as an aryl group of any one of the formulae:

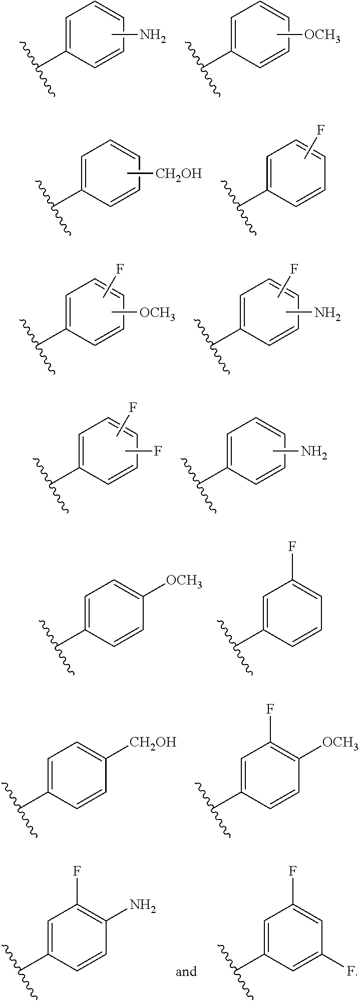

Alternatively, or in addition, in any of the compounds of the formulae (I) and (Ia)-(If), R² can be (C₁-C₆)alkyl or R² can be (C₁-C₃)alkyl, an example of each of which is (C₃)alkyl.

Examples of compounds of the formulae (I) and (Ia)-(If) include the following:

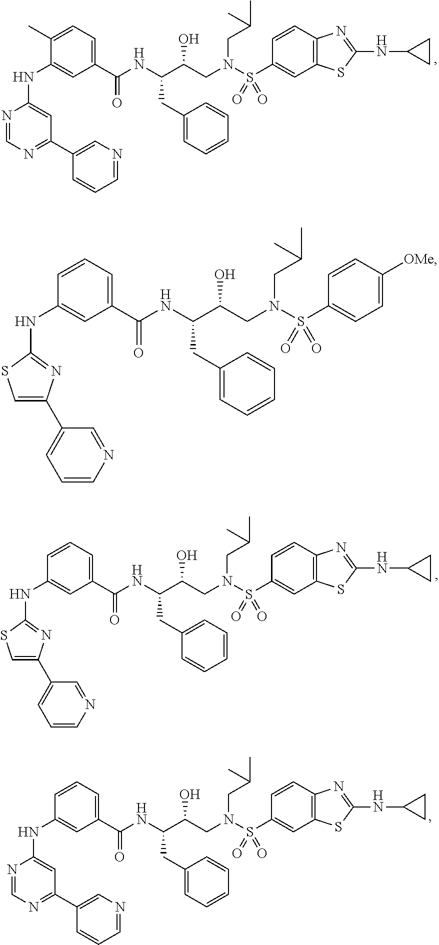 [SMILES: Cc1ccc(C(=O)N[C@@H](Cc2ccccc2)[C@H](O)CN(CC(C)C)S(=O)(=O)c2ccc3nc(NC4CC4)sc3c2)cc1Nc1cc(-c2cccnc2)ncn1 ; COc1ccc(S(=O)(=O)N(CC(C)C)C[C@@H](O)[C@H](Cc2ccccc2)NC(=O)c2cccc(Nc3nc(-c4cccnc4)cs3)c2)cc1 ; CC(C)CN(C[C@@H](O)[C@H](Cc1ccccc1)NC(=O)c1cccc(Nc2nc(-c3cccnc3)cs2)c1)S(=O)(=O)c1ccc2nc(NC3CC3)sc2c1 ; CC(C)CN(C[C@@H](O)[C@H](Cc1ccccc1)NC(=O)c1cccc(Nc2cc(-c3cccnc3)ncn2)c1)S(=O)(=O)c1ccc2nc(NC3CC3)sc2c1]

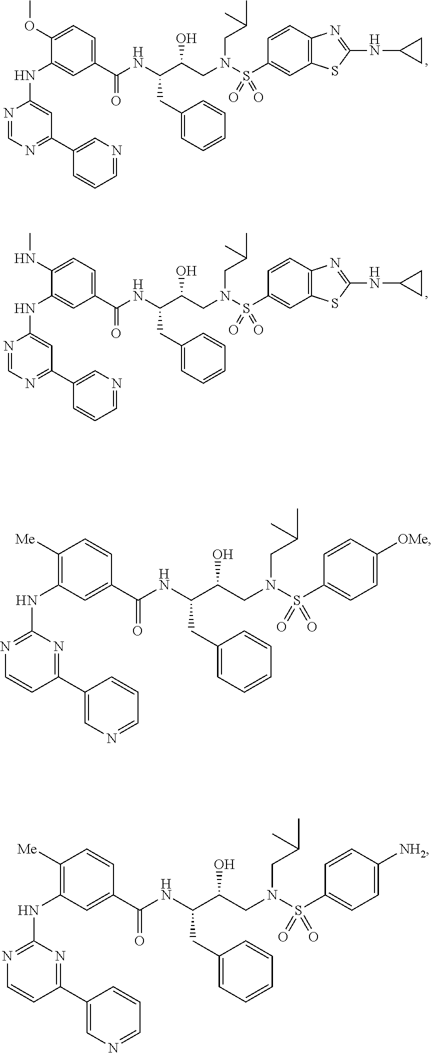 [SMILES: COc1ccc(C(=O)N[C@@H](Cc2ccccc2)[C@H](O)CN(CC(C)C)S(=O)(=O)c2ccc3nc(NC4CC4)sc3c2)cc1Nc1cc(-c2cccnc2)ncn1 ; CNc1ccc(C(=O)N[C@@H](Cc2ccccc2)[C@H](O)CN(CC(C)C)S(=O)(=O)c2ccc3nc(NC4CC4)sc3c2)cc1Nc1cc(-c2cccnc2)ncn1 ; COc1ccc(S(=O)(=O)N(CC(C)C)C[C@@H](O)[C@H](Cc2ccccc2)NC(=O)c2ccc(C)c(Nc3nccc(-c4cccnc4)n3)c2)cc1 ; Cc1ccc(C(=O)N[C@@H](Cc2ccccc2)[C@H](O)CN(CC(C)C)S(=O)(=O)c2ccc(N)cc2)cc1Nc1nccc(-c2cccnc2)n1]

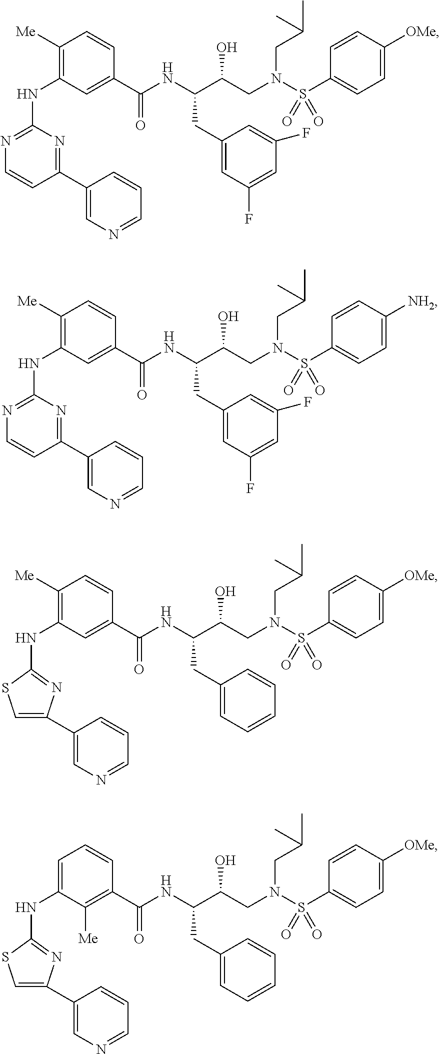 [SMILES: COc1ccc(S(=O)(=O)N(CC(C)C)C[C@@H](O)[C@H](Cc2cc(F)cc(F)c2)NC(=O)c2ccc(C)c(Nc3nccc(-c4cccnc4)n3)c2)cc1 ; Cc1ccc(C(=O)N[C@@H](Cc2cc(F)cc(F)c2)[C@H](O)CN(CC(C)C)S(=O)(=O)c2ccc(N)cc2)cc1Nc1nccc(-c2cccnc2)n1 ; COc1ccc(S(=O)(=O)N(CC(C)C)C[C@@H](O)[C@H](Cc2ccccc2)NC(=O)c2ccc(C)c(Nc3nc(-c4cccnc4)cs3)c2)cc1 ; COc1ccc(S(=O)(=O)N(CC(C)C)C[C@@H](O)[C@H](Cc2ccccc2)NC(=O)c2cccc(Nc3nc(-c4cccnc4)cs3)c2C)cc1]

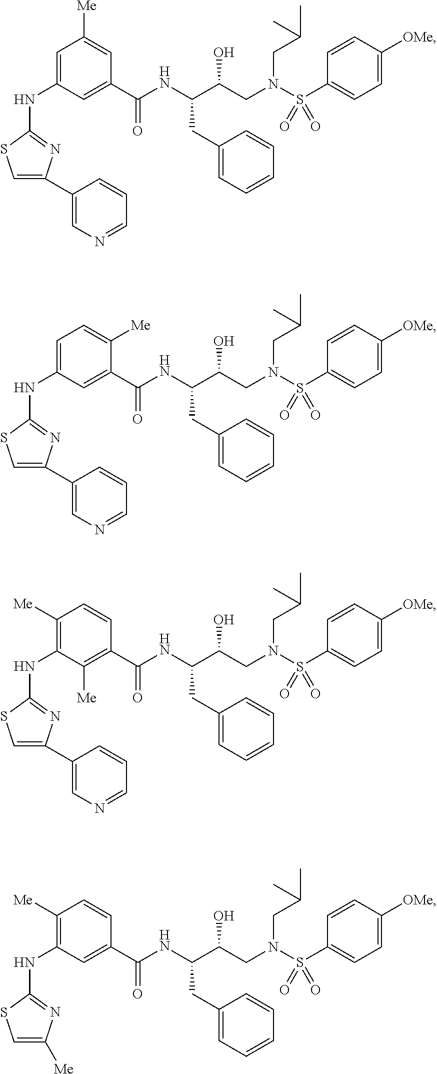 [SMILES: COc1ccc(S(=O)(=O)N(CC(C)C)C[C@@H](O)[C@H](Cc2ccccc2)NC(=O)c2cc(C)cc(Nc3nc(-c4cccnc4)cs3)c2)cc1 ; COc1ccc(S(=O)(=O)N(CC(C)C)C[C@@H](O)[C@H](Cc2ccccc2)NC(=O)c2cc(Nc3nc(-c4cccnc4)cs3)ccc2C)cc1 ; COc1ccc(S(=O)(=O)N(CC(C)C)C[C@@H](O)[C@H](Cc2ccccc2)NC(=O)c2ccc(C)c(Nc3nc(-c4cccnc4)cs3)c2C)cc1 ; COc1ccc(S(=O)(=O)N(CC(C)C)C[C@@H](O)[C@H](Cc2ccccc2)NC(=O)c2ccc(C)c(Nc3nc(C)cs3)c2)cc1]

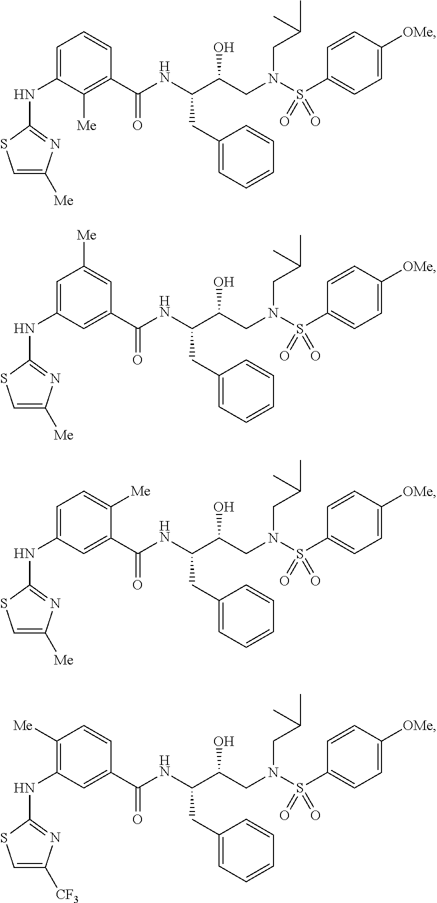

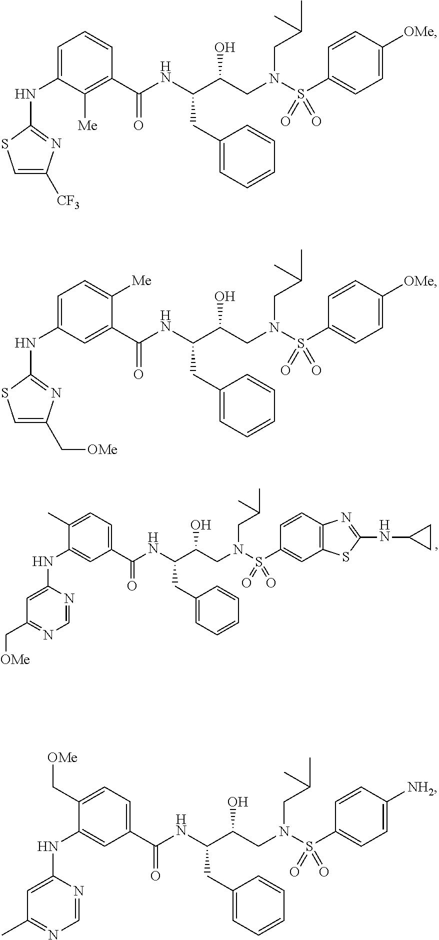 [SMILES: COc1ccc(S(=O)(=O)N(CC(C)C)C[C@@H](O)[C@H](Cc2ccccc2)NC(=O)c2cccc(Nc3nc(C(F)(F)F)cs3)c2C)cc1 ; COCc1csc(Nc2ccc(C)c(C(=O)N[C@@H](Cc3ccccc3)[C@H](O)CN(CC(C)C)S(=O)(=O)c3ccc(OC)cc3)c2)n1 ; COCc1cc(Nc2cc(C(=O)N[C@@H](Cc3ccccc3)[C@H](O)CN(CC(C)C)S(=O)(=O)c3ccc4nc(NC5CC5)sc4c3)ccc2C)ncn1 ; COCc1ccc(C(=O)N[C@@H](Cc2ccccc2)[C@H](O)CN(CC(C)C)S(=O)(=O)c2ccc(N)cc2)cc1Nc1cc(C)ncn1]

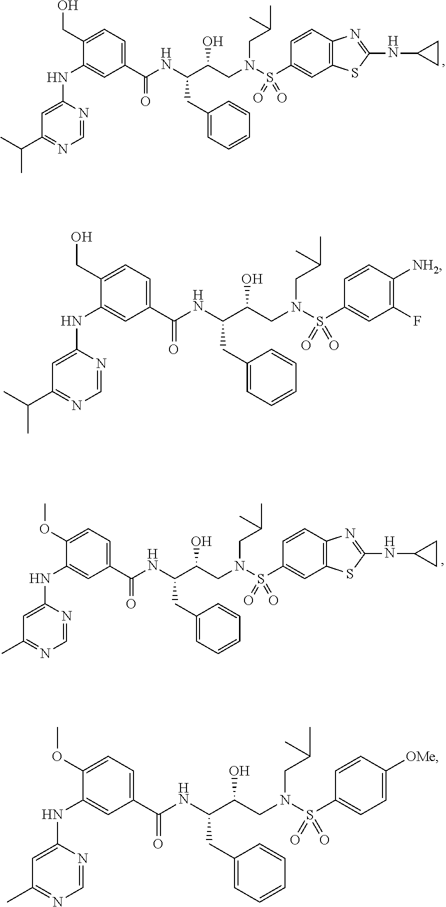 [SMILES: CC(C)CN(C[C@@H](O)[C@H](Cc1ccccc1)NC(=O)c1ccc(CO)c(Nc2cc(C(C)C)ncn2)c1)S(=O)(=O)c1ccc2nc(NC3CC3)sc2c1 ; CC(C)CN(C[C@@H](O)[C@H](Cc1ccccc1)NC(=O)c1ccc(CO)c(Nc2cc(C(C)C)ncn2)c1)S(=O)(=O)c1ccc(N)c(F)c1 ; COc1ccc(C(=O)N[C@@H](Cc2ccccc2)[C@H](O)CN(CC(C)C)S(=O)(=O)c2ccc3nc(NC4CC4)sc3c2)cc1Nc1cc(C)ncn1 ; COc1ccc(S(=O)(=O)N(CC(C)C)C[C@@H](O)[C@H](Cc2ccccc2)NC(=O)c2ccc(OC)c(Nc3cc(C)ncn3)c2)cc1]

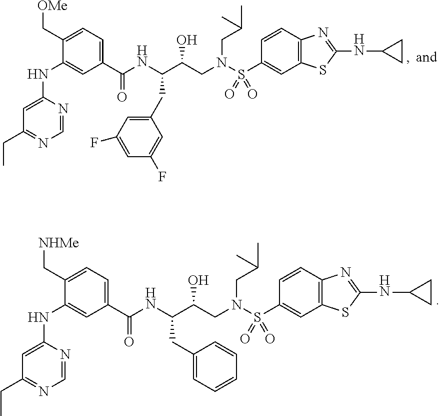 [SMILES: CCc1cc(Nc2cc(C(=O)N[C@@H](Cc3cc(F)cc(F)c3)[C@H](O)CN(CC(C)C)S(=O)(=O)c3ccc4nc(NC5CC5)sc4c3)ccc2COC)ncn1 ; CCc1cc(Nc2cc(C(=O)N[C@@H](Cc3ccccc3)[C@H](O)CN(CC(C)C)S(=O)(=O)c3ccc4nc(NC5CC5)sc4c3)ccc2CNC)ncn1]

Those of ordinary skill in the art will recognize that compounds described herein (e.g., the compounds of the formula (I) and (la)-(If)) contain chiral centers. All diastereomers of the compounds described herein are contemplated herein, as well as racemates. Thus, for example, the compounds of formula (I) and (la)-(If) are contemplated herein, as well as racemates.

The compounds can be synthesized in accordance with methods known in the art. Methods of synthesis are also exemplified herein (see Examples).

The present disclosure also provides a pharmaceutical composition comprising a compound of any of the preceding formulae and a pharmaceutically acceptable excipient. The present disclosure also provides a pharmaceutical composition comprising a therapeutically effective amount of one or more compounds of the formula (I) and (la)-(If), and a pharmaceutically acceptable excipient.

Pharmaceutical compositions contemplated herein are those comprising one or more compounds of the various embodiments of the present disclosure and one or more pharmaceutically acceptable excipients. A “pharmaceutical composition” refers to a chemical or biological composition suitable for administration to a subject (e.g., mammal). Such compositions can be specifically formulated for administration via one or more of a number of routes including, but not limited to, buccal, cutaneous, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. In addition, administration can by means of capsule, drops, foams, gel, gum, injection, liquid, patch, pill, porous pouch, powder, tablet, or other suitable means of administration.

A “pharmaceutical excipient” or a “pharmaceutically acceptable excipient” is a carrier, sometimes a liquid, in which an active therapeutic agent is formulated. The excipient generally does not provide any pharmacological activity to the formulation, though it can provide chemical and/or biological stability, and release characteristics. Examples of suitable formulations can be found, for example, in Remington, The Science and Practice of Pharmacy, 20th Edition, (Gennaro, A. R., Chief Editor), Philadelphia College of Pharmacy and Science, 2000, which is incorporated by reference in its entirety.

As used herein “pharmaceutically acceptable carrier” or “excipient” includes, but is not limited to, any and all solvents, dispersion media, coatings, antibacterial and antifungal agents, and isotonic and absorption delaying agents that are physiologically compatible. In one embodiment, the carrier is suitable for parenteral administration. Alternatively, the carrier can be suitable for intravenous, intraperitoneal, intramuscular, sublingual, or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well-known in the art. Except insofar as any conventional media or agent is incompatible with the active compound, use thereof in the pharmaceutical compositions is contemplated. Supplementary active compounds can also be incorporated into the compositions.

Pharmaceutical compositions can be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (e.g., glycerol, propylene glycol, and liquid polyethylene glycol), and suitable mixtures thereof. The proper fluidity can be maintained, for example, by the use of a coating such as lecithin, by the maintenance of the required particle size in the case of dispersion, and by the use of surfactants.

In many cases, it will be preferable to include isotonic agents, for example, sugars, polyalcohols such as mannitol, sorbitol, or sodium chloride in the composition. Prolonged absorption of injectable compositions can be brought about by including in the composition an agent which delays absorption, for example, monostearate salts and gelatin. Moreover, the compounds can be formulated in a time-release formulation, for example in a composition that includes a slow-release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, and polyglycolic copolymers (PLG). Many methods for the preparation of such formulations are known to those skilled in the art.

Oral forms of administration are also contemplated herein. The pharmaceutical compositions can be orally administered as a capsule (hard or soft), tablet (film coated, enteric coated or uncoated), powder or granules (coated or uncoated) or liquid (solution or suspension). The formulations can be conveniently prepared by any of the methods well-known in the art. The pharmaceutical compositions can include one or more suitable production aids or excipients including fillers, binders, disintegrants, lubricants, diluents, flow agents, buffering agents, moistening agents, preservatives, colorants, sweeteners, flavors, and pharmaceutically compatible carriers.

For each of the recited embodiments, the compounds can be administered by a variety of dosage forms as known in the art. Any biologically acceptable dosage form known to persons of ordinary skill in the art, and combinations thereof, are contemplated. Examples of such dosage forms include, without limitation, chewable tablets, quick-dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, gum, granules, particles, microparticles, dispersible granules, cachets, douches, suppositories, creams, topicals, inhalants, aerosol inhalants, patches, particle inhalants, implants, depot implants, ingestibles, injectables (including subcutaneous, intramuscular, intravenous, and intradermal), infusions, and combinations thereof.

Other compounds, which can be included by admixture, are, for example, medically inert ingredients (e.g., solid and liquid diluent), such as lactose, dextrose-saccharose, cellulose, starch or calcium phosphate for tablets or capsules; olive oil or ethyl oleate for soft capsules and water or vegetable oil for suspensions or emulsions; lubricating agents such as silica, talc, stearic acid, magnesium or calcium stearate and/or polyethylene glycols; gelling agents such as colloidal clays; thickening agents such as gum tragacanth or sodium alginate; binding agents such as starches, arabic gums, gelatin, methylcellulose, carboxymethylcellulose or polyvinylpyrrolidone; disintegrating agents such as starch, alginic acid, alginates or sodium starch glycolate; effervescing mixtures; dyestuff; sweeteners; wetting agents such as lecithin, polysorbates or laurylsulphates; and other therapeutically acceptable accessory ingredients, such as humectants, preservatives, buffers and antioxidants, which are known additives for such formulations.

Liquid dispersions for oral administration can be syrups, emulsions, solutions, or suspensions. The syrups can contain as a carrier, for example, saccharose or saccharose with glycerol and/or mannitol and/or sorbitol. The suspensions and the emulsions can contain a carrier, for example a natural gum, agar, sodium alginate, pectin, methylcellulose, carboxymethylcellulose, or polyvinyl alcohol.

The amount of active compound in a therapeutic composition according to various embodiments of the present invention can vary according to factors such as the disease state, age, gender, weight, patient history, risk factors, predisposition to disease, administration route, and pre-existing treatment regime (e.g., possible interactions with other medications) of the subject. Dosage regimens can be adjusted to provide the optimum therapeutic response. For example, a single bolus can be administered, several divided doses can be administered over time, or the dose can be proportionally reduced or increased as indicated by the exigencies of therapeutic situation.

A “dosage unit form” refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms is dictated by and directly dependent on the unique characteristics of the active compound, the particular therapeutic effect to be achieved, and the limitations inherent in the art of compounding such an active compound. In therapeutic use for treatment of conditions in mammals (e.g., humans) for which the compounds or appropriate pharmaceutical compositions thereof are effective, the compounds can be administered in an effective amount. The dosage can be a composition, a pharmaceutical composition, or any other composition.

For each of the recited embodiments, the dosage is typically administered once, twice, or thrice a day, although more frequent dosing intervals are possible. The dosage can be administered every day, every 2 days, every 3 days, every 4 days, every 5 days, every 6 days, and/or every 7 days (once a week). In one embodiment, the dosage can be administered daily for up to and including 30 days, preferably between 7-10 days. In another embodiment, the dosage can be administered twice a day for 10 days. If the patient requires treatment for a chronic disease or condition, the dosage can be administered for as long as signs and/or symptoms persist. The patient can require “maintenance treatment” where the patient is receiving dosages every day for months, years, or the remainder of his life. In addition, the composition can effect prophylaxis of recurring symptoms. For example, the dosage can be administered once or twice a day to prevent the onset of symptoms in patients at risk, especially for asymptomatic patients.

The compositions can be administered in any of the following routes: buccal, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. The preferred routes of administration are buccal and oral. The administration can be local, where the composition is administered directly, close to, in the locality, near, at, about, or in the vicinity of, the site(s) of disease, e.g., inflammation, or systemic, wherein the composition is given to the patient and passes through the body widely, thereby reaching the site(s) of disease. Local administration can be administration to, for example, tissue, organ, and/or organ system, which encompasses and/or is affected by the disease, and/or where the disease signs and/or symptoms are active or are likely to occur. Administration can be topical with a local effect, e.g., the composition is applied directly where its action is desired. Administration can be enteral with a systemic (non-local) effect, e.g., the composition is given via the digestive tract or another parenteral route.

The disclosure also relates to compositions comprising a therapeutically effective amount of one or more compounds (e.g. a compound of the formula (I) and (Ia)-(If)). Such compositions are useful in a method for treating an HIV (e.g., HIV-1) infection or AIDS, the method comprising administering a therapeutically effective amount of one or more compounds to a patient in need thereof.

The term “therapeutically effective amount” refers to that amount of one or more compounds (e.g. a compound of the formula (I) and (Ia)-(If)) that elicits a biological or medicinal response in a tissue system, animal or human, that is being sought by a researcher, veterinarian, medical doctor or other clinician, which includes alleviation of the symptoms of the disease or disorder being treated. In some embodiments, the therapeutically effective amount is that which may treat or alleviate the disease or symptoms of the disease at a reasonable benefit/risk ratio applicable to any medical treatment. However, it is to be understood that the total daily usage of the compounds and compositions may be decided by the attending physician within the scope of sound medical judgment. The specific therapeutically effective dose level for any particular patient will depend upon a variety of factors, including the condition being treated and the severity of the condition; activity of the specific compound employed; the specific composition employed; the age, body weight, general health, gender and diet of the patient; the time of administration, route of administration, and rate of excretion of the specific compound employed; the duration of the treatment; drugs used in combination or coincidentally with the specific compound employed; and like factors well known to the researcher, veterinarian, medical doctor or other clinician. It is also appreciated that the therapeutically effective amount can be selected with reference to any toxicity, or other undesirable side effect, that might occur during administration of one or more of the compounds.

The compounds can and do have an HIV-1 protease inhibition constant (Kᵢ) of from about 1 μM to about 200 nM (e.g., about 1 μM to about 100 nM, about 1 pM to about 100 μM, about 5 μM to about 100 μM, about 50 μM to about 250 μM, about 500 μM to about 100 nM or about 300 PM to about 75 nM). In addition or alternatively, the compounds can and do have an antiviral activity in vitro against a wild-type laboratory strain, HIV-1_{LAI}, with half-maximal inhibitory concentration (IC₅₀) of from about 1 nM to about 600 nM (e.g., about 1 nM to 600 nM, 1 nM to about 600 nM, 1 nM to 600 nM, about 1 nM to about 100 nM (e.g., about 1 to 100 nM, 1 to about 100 nM, or 1 to 100 nM), about 1 nM to about 200 nM (e.g., about 1 to 200 nM, 1 to about 200 nM, or 1 to 200 nM), about 100 nM to about 200 nM (e.g., about 1 to 200 nM, 1 to about 200 nM, or 1 to 200 nM), about 10 nM to about 50 nM (e.g., about 10 nM to 50 nM, 10 nM to about 50 nM, or 10 to 50 nM) or about 10 nM to about 500 nM (e.g., about 10 nM to 500 nM, 10 nM to about 500 nM, or 10 nM to 500 nM)). In addition or alternatively, the compounds can and do have antiviral activity in vitro against one or more darunavir-resistant HIV-1 variants (e.g., NL4-3R, DRV_{R}P20, DRV_{R}P30, and DRV_{R}P51) with an IC₅₀ of from about 1 nM to about 600 nM (e.g., about 1 nM to 600 nM, 1 nM to about 600 nM 1 nM to 600 nM, about 1 nM to about 100 nM (e.g., about 1 to 100 nM, 1 to about 100 nM, or 1 to 100 nM), about 1 nM to about 200 nM (e.g., about 1 nM to 200 nM, 1 nM to about 200 nM, or 1 nM to 200 nM), about 100 nM to about 200 nM (e.g., about 100 nM to 200 nM, 100 nM to about 200 nM, or 100 nM to 200 nM), 10 nM to about 50 nM (e.g., about 10 nM to 50 nM, 10 nM to about 50 nM, or 10 nM to 50 nM) or about 10 nM to about 500 nM (e.g., about 10 nM to 500 nM, 10 nM to about 500 nM, or 10 nM to 500 nM)). In addition or alternatively, the compounds can and do have antiviral activity in vitro against one or more darunavir-resistant HIV-1 variants (e.g., NL4-3R, DRV_{R}P20, DRV_{R}P30, and DRV_{R}P51) with an IC₅₀ of from about 50 μM to about 50 nM (e.g., about 50 μM to 50 nM, 50 PM to about 50 nM, 50 μM to 50 nM, about 1 μM to about 100 nM (e.g., about 1 μM to 100 nM, 1 μM to about 100 nM, or 1 μM to 100 nM), about 100 μM to about 50 nM (e.g., about 100 μM to 50 nM, 100 μM to about 50 nM, or 100 μM to 50 nM) or about 500 μM to about 10 nM (e.g., about 500 μM to 10 nM, 500 PM to about 10 nM, or 500 μM to 10 nM)). In addition or alternatively, the compounds can and do have antiviral activity in vitro against one or more darunavir-resistant HIV-1 proteases (e.g., NL4-3R, DRV_{R}P20, DRV_{R}P30, and DRV_{R}P51) with an IC₅₀ of from about 1 nM to about 600 nM (e.g., about 1 nM to 600 nM, 1 nM to about 600 nM, 1 nM to 600 nM, about 1 nM to about 100 nM (e.g., about 1 nM to 100 nM, 1 nM to about 100 nM, or 1 nM to 100 nM), about 1 nM to about 200 nM (e.g., about 1 nM to 200 nM, 1 nM to about 200 nM, or 1 nM to 200 nM), about 100 nM to about 200 nM (e.g., about 100 nM to 200 nM, 100 nM to about 200 nM, or 100 nM to 200 nM), 10 nM to about 50 nM (e.g., about 10 nM to 50 nM, 10 nM to about 50 nM, or 10 nM to 50 nM), or about 10 nM to about 500 nM (e.g., about 10 nM to 500 nM, 10 nM to about 500 nM, or 10 nM to 500 nM)).

The term “alkyl” refers to substituted or unsubstituted straight chain, branched and cyclic, saturated mono- or bi-valent groups, which can be referred to herein as “alkylene,” having from 1 to 20 carbon atoms, 10 to 20 carbon atoms, 12 to 18 carbon atoms, 6 to about 10 carbon atoms, 1 to 10 carbons atoms, 1 to 8 carbon atoms, 2 to 8 carbon atoms, 3 to 8 carbon atoms, 4 to 8 carbon atoms, 5 to 8 carbon atoms, 1 to 6 carbon atoms, 2 to 6 carbon atoms, 3 to 6 carbon atoms, or 1 to 3 carbon atoms. Examples of straight chain mono-valent (C₁-C₂₀)-alkyl groups include those with from 1 to 8 carbon atoms such as methyl (i.e., CH₃), ethyl, n-propyl, n-butyl, n-pentyl, n-hexyl, n-heptyl, and n-octyl groups. Examples of branched mono-valent (C₁-C₂₀)-alkyl groups include isopropyl, iso-butyl, sec-butyl, t-butyl, neopentyl, and isopentyl. Examples of straight chain bi-valent (C₁-C₂₀)alkyl groups include those with from 1 to 6 carbon atoms such as —CH₂—, —CH₂CH₂—, —CH₂CH₂CH₂—, —CH₂CH₂CH₂CH₂—, and —CH₂CH₂CH₂CH₂CH₂—. Examples of branched bi-valent alkyl groups include —CH(CH₃)CH₂— and —CH₂CH(CH₃)CH₂—. Examples of cyclic alkyl groups include cyclopropyl, cyclobutyl, cyclohexyl, cyclopently, cyclooctyl, bicyclo[1.1.1]pentyl, bicyclo[2.1.1]hexyl, and bicyclo[2.2.1]heptyl. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. In some embodiments, alkyl includes a combination of substituted and unsubstituted alkyl. As an example, alkyl, and also (C₁)alkyl, includes methyl and substituted methyl. As a particular example, (C₁)alkyl includes benzyl. As a further example, alkyl can include methyl and substituted (C₂-C₅)alkyl. Alkyl can also include substituted methyl and unsubstituted (C₂-C₈)alkyl. In some embodiments, alkyl can be methyl and C₂-C₈ linear alkyl. In some embodiments, alkyl can be methyl and C₂-C₈ branched alkyl. The term methyl is understood to be —CH₃, which is not substituted. The term methylene is understood to be —CH₂—, which is not substituted. For comparison, the term (C₁)alkyl is understood to be a substituted or an unsubstituted —CH₃ or a substituted or an unsubstituted —CH₂—. Representative substituted alkyl groups can be substituted one or more times with any of the groups listed herein, for example, cycloalkyl, heterocyclyl, aryl, amino, haloalkyl, hydroxy, cyano, carboxy, nitro, thio, alkoxy, and halogen groups. As further examples, representative substituted alkyl groups can be substituted with one or more fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. Examples of substituted alkyl groups include alkyl groups substituted with halo, amino, hydroxy, cyano, carboxy, nitro, thio and alkoxy. For example, representative substituted alkyl groups can be substituted with a fluoro group. In some embodiments, representative substituted alkyl groups can be substituted with one, two, three or more fluoro groups or they can be substituted with one, two, three or more non-fluoro groups. For example, alkyl can be trifluoromethyl, difluoromethyl, or fluoromethyl, or alkyl can be substituted alkyl other than trifluoromethyl, difluoromethyl or fluoromethyl. Alkyl can be haloalkyl or alkyl can be a substituted alkyl other than haloalkyl. The term “alkyl” also generally refers to alkyl groups that can comprise one or more heteroatoms in the carbon chain, including nitrogen and oxygen. Thus, for example, “alkyl” also encompasses groups such as —(CH₂)ₚO]_{q}H and the like.

The term “alkenyl” refers to substituted or unsubstituted straight chain, branched and cyclic, saturated mono- or bi-valent groups having at least one carbon-carbon double bond and from 2 to 20 carbon atoms, 10 to 20 carbon atoms, 12 to 18 carbon atoms, 6 to about 10 carbon atoms, 2 to 10 carbons atoms, 2 to 8 carbon atoms, 3 to 8 carbon atoms, 4 to 8 carbon atoms, 5 to 8 carbon atoms, 2 to 6 carbon atoms, 3 to 6 carbon atoms, 4 to 6 carbon atoms, 2 to 4 carbon atoms, or 2 to 3 carbon atoms. The double bonds can be trans or cis orientation. The double bonds can be terminal or internal. The alkenyl group can be attached via the portion of the alkenyl group containing the double bond, e.g., vinyl, propen-1-yl and buten-1-yl, or the alkenyl group can be attached via a portion of the alkenyl group that does not contain the double bond, e.g., penten-4-yl. Examples of mono-valent (C₂-C₂₀)-alkenyl groups include those with from 1 to 8 carbon atoms such as vinyl, propenyl, propen-1-yl, propen-2-yl, butenyl, buten-1-yl, buten-2-yl, sec-buten-1-yl, sec-buten-3-yl, pentenyl, hexenyl, heptenyl and octenyl groups. Examples of branched mono-valent (C₂-C₂₀)-alkenyl groups include isopropenyl, iso-butenyl, sec-butenyl, t-butenyl, neopentenyl, and isopentenyl. Examples of straight chain bi-valent (C₂-C₂₀) alkenyl groups include those with from 2 to 6 carbon atoms such as —CHCH—, —CHCHCH₂—, —CHCHCH₂CH₂—, and —CHCHCH₂CH₂CH₂—. Examples of branched bi-valent alkyl groups include —C(CH₃)CH— and —CHC(CH₃) CH₂—. Examples of cyclic alkenyl groups include cyclopentenyl, cyclohexenyl and cyclooctenyl. It is envisaged that alkenyl can also include masked alkenyl groups, precursors of alkenyl groups or other related groups. As such, where alkenyl groups are described it, compounds are also envisaged where a carbon-carbon double bond of an alkenyl is replaced by an epoxide or aziridine ring. Substituted alkenyl also includes alkenyl groups which are substantially tautomeric with a non-alkenyl group. For example, substituted alkenyl can be 2-aminoalkenyl, 2-alkylaminoalkenyl, 2-hydroxyalkenyl, 2-hydroxyvinyl, 2-hydroxypropenyl, but substituted alkenyl is also understood to include the group of substituted alkenyl groups other than alkenyl which are tautomeric with non-alkenyl containing groups. In some embodiments, alkenyl can be understood to include a combination of substituted and unsubstituted alkenyl. For example, alkenyl can be vinyl and substituted vinyl. For example, alkenyl can be vinyl and substituted (C₃-C₅)alkenyl. Alkenyl can also include substituted vinyl and unsubstituted (C₃-C₅)alkenyl. Representative substituted alkenyl groups can be substituted one or more times with any of the groups listed herein, for example, monoalkylamino, dialkylamino, cyano, acetyl, amido, carboxy, nitro, alkylthio, alkoxy, and halogen groups. As further examples, representative substituted alkenyl groups can be substituted one or more fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. In some embodiments, representative substituted alkenyl groups can be substituted from a set of groups including monoalkylamino, dialkylamino, cyano, acetyl, amido, carboxy, nitro, alkylthio and alkoxy, but not including halogen groups. Thus, in some embodiments, alkenyl can be substituted with a non-halogen group. In some embodiments, representative substituted alkenyl groups can be substituted with a fluoro group, a bromo group, a halogen other than bromo, or a halogen other than fluoro. For example, alkenyl can be 1-fluorovinyl, 2-fluorovinyl, 1,2-difluorovinyl, 1,2,2-trifluorovinyl, 2,2-difluorovinyl, trifluoropropen-2-yl, 3,3,3-trifluoropropenyl, 1-fluoropropenyl, 1-chlorovinyl, 2-chlorovinyl, 1,2-dichlorovinyl, 1,2,2-trichlorovinyl or 2,2-dichlorovinyl. In some embodiments, representative substituted alkenyl groups can be substituted with one, two, three or more fluoro groups or they can be substituted with one, two, three or more non-fluoro groups.

The term “alkynyl” refers to substituted or unsubstituted straight and branched chain alkyl groups, except that at least one triple bond exists between two carbon atoms. Thus, alkynyl groups have from 2 to 50 carbon atoms, 2 to 20 carbon atoms, 10 to 20 carbon atoms, 12 to 18 carbon atoms, 6 to about 10 carbon atoms, 2 to 10 carbons atoms, 2 to 8 carbon atoms, 3 to 8 carbon atoms, 4 to 8 carbon atoms, 5 to 8 carbon atoms, 2 to 6 carbon atoms, 3 to 6 carbon atoms, 4 to 6 carbon atoms, 2 to 4 carbon atoms, or 2 to 3 carbon atoms. Examples include, but are not limited to, ethynyl, propynyl, propyn-1-yl, propyn-2-yl, butynyl, butyn-1-yl, butyn-2-yl, butyn-3-yl, butyn-4-yl, pentynyl, pentyn-1-yl, hexynyl, Examples include, but are not limited to —C═CH, —C═C(CH₃), —C═C(CH₂CH₃), —CH₂C═CH, —CH₂C═C(CH₃), and —CH₂C═C(CH₂CH₃) among others.

The term “aryl” refers to substituted or unsubstituted univalent groups that are derived by removing a hydrogen atom from an arene, which is a cyclic aromatic hydrocarbon, having from 6 to 20 carbon atoms, 10 to 20 carbon atoms, 12 to 20 carbon atoms, 6 to about 10 carbon atoms or 6 to 8 carbon atoms. Examples of (C₆-C₂₀) aryl groups include phenyl, napthalenyl, azulenyl, biphenylyl, indacenyl, fluorenyl, phenanthrenyl, triphenylenyl, pyrenyl, naphthacenyl, chrysenyl, and anthracenyl groups. Other examples include substituted phenyl, substituted napthalenyl, substituted azulenyl, substituted biphenylyl, substituted indacenyl, substituted fluorenyl, substituted phenanthrenyl, substituted triphenylenyl, substituted pyrenyl, substituted naphthacenyl, substituted chrysenyl, and substituted anthracenyl groups. Examples also include unsubstituted phenyl, unsubstituted napthalenyl, unsubstituted azulenyl, unsubstituted biphenylyl, unsubstituted indacenyl, unsubstituted fluorenyl, unsubstituted phenanthrenyl, unsubstituted triphenylenyl, unsubstituted pyrenyl, unsubstituted naphthacenyl, unsubstituted chrysenyl, and unsubstituted anthracenyl groups. Aryl includes phenyl groups and also non-phenyl aryl groups. From these examples, it is clear that the term (C₆-C₂₀) aryl encompasses mono- and polycyclic (C₆-C₂₀) aryl groups, including fused and non-fused polycyclic (C₆-C₂₀) aryl groups.

The term “heterocyclyl” refers to substituted aromatic, unsubstituted aromatic, substituted non-aromatic, and unsubstituted non-aromatic rings containing 3 or more atoms in the ring, of which one or more is a heteroatom such as, but not limited to, N, O, and S. Thus, a heterocyclyl can be a cycloheteroalkyl or a heteroaryl, or if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₈), 3 to 6 carbon atoms (C₃-C₆) or 6 to 8 carbon atoms (C₆-C₈). A heterocyclyl group designated as a C₂-heterocyclyl can be a 5-membered ring with two carbon atoms and three heteroatoms, a 6-membered ring with two carbon atoms and four heteroatoms and so forth. Likewise, a C₄-heterocyclyl can be a 5-membered ring with one heteroatom, a 6-membered ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. Representative heterocyclyl groups include, but are not limited to. piperidynyl, piperazinyl, morpholinyl, furanyl, pyrrolidinyl, pyridinyl, pyrazinyl, pyrimidinyl, triazinyl, thiophenyl, tetrahydrofuranyl, pyrrolyl, oxazolyl, imidazolyl, triazyolyl, tetrazolyl, benzoxazolinyl, and benzimidazolinyl groups. For example, heterocyclyl groups include, without limitation:

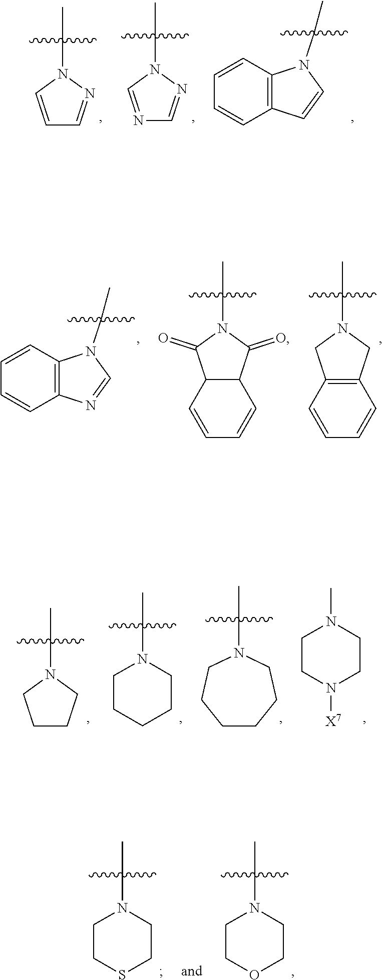

wherein X⁷ represents H, (C₁-C₂₀)alkyl, (C₆-C₂₀) aryl or an amine protecting group (e.g., a t-butyloxycarbonyl group) and wherein the heterocyclyl group can be substituted or unsubstituted. A nitrogen-containing heterocyclyl group is a heterocyclyl group containing a nitrogen atom as an atom in the ring. In some embodiments, the heterocyclyl is other than thiophene or substituted thiophene. In some embodiments, the heterocyclyl is other than furan or substituted furan.

The term “alkoxy” as used herein refers to an oxygen atom connected to an alkyl group, including a cycloalkyl group, as are defined herein. Examples of linear alkoxy groups include but are not limited to, methoxy, ethoxy, propoxy, butoxy, pentyloxy, hexyloxy, and the like. Examples of branched alkoxy include, but are not limited to, isopropoxy, sec-butoxy, tert-butoxy, isopentyloxy, isohexyloxy, and the like. Examples of cyclic alkoxy include, but are not limited to, cyclopropyloxy, cyclobutyloxy, cyclopentyloxy, cyclohexyloxy, and the like. An alkoxy group can include one to about 12-20 or about 12-40 carbon atoms bonded to the oxygen atom, and can further include double or triple bonds, and can also include heteroatoms. Thus, alkyoxy also includes an oxygen atom connected to an alkyenyl group and oxygen atom connected to an alkynyl group. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “aryloxy” refers to an oxygen atom connected to an aryl group as defined herein.

The terms “aralkyl” and “arylalkyl” refer to alkyl groups as defined herein in which a hydrogen or carbon bond of an alkyl group is replaced with a bond to an aryl group as defined herein. Representative aralkyl groups include benzyl, biphenylmethyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The terms “halo,” “halogen,” and “halide” group, by themselves or as part of another substituent, mean, unless otherwise stated, a fluorine, chlorine, bromine, or iodine atom.

The terms “amine” and “amino” refer to a substituent of the form —NH₂, —NHR, —NR², or —NR₃⁺, wherein each R is independently selected, and protonated forms of each, except for —NR₃⁺, which cannot be protonated. Accordingly, any compound substituted with an amino group can be viewed as an amine. An “amino group” can be a primary, secondary, tertiary, or quaternary amino group. An “alkylamino” group includes a monoalkylamino group, a dialkylamino group, and a trialkylamino group.

The term “acyl” refers to a group containing a carbonyl moiety wherein the group is bonded via the carbonyl carbon atom. The carbonyl carbon atom is also bonded to another carbon atom, which can be part of a substituted or unsubstituted alkyl, alkenyl, alkynyl, aryl, cycloalkyl, heterocyclyl, group or the like.

The term “formyl” refers to a group containing a carbonyl moiety wherein the group is bonded via the carbonyl carbon atom. The carbonyl carbon atom is also bonded to a hydrogen atom.

The term “alkoxycarbonyl” refers to a group containing a carbonyl moiety wherein the group is bonded via the carbonyl carbon atom. The carbonyl carbon atom is also bonded to an oxygen atom which is further bonded to an alkyl group. Alkoxycarbonyl also includes the group where a carbonyl carbon atom is also bonded to an oxygen atom which is further bonded to an alkyenyl group.

Alkoxycarbonyl also includes the group where a carbonyl carbon atom is also bonded to an oxygen atom which is further bonded to an alkynyl group. In a further case, which is included in the definition of alkoxycarbonyl, and included in the term “aryloxycarbonyl,” the carbonyl carbon atom is bonded to an oxygen atom, which is bonded to an aryl group instead of an alkyl group.

The term “arylcarbonyl” refers to a group containing a carbonyl moiety wherein the group is bonded via the carbonyl carbon atom. The carbonyl carbon atom is also bonded to an aryl group.

The term “alkylamido” refers to a group containing a carbonyl moiety wherein the group is bonded via the carbonyl carbon atom. The carbonyl carbon atom is also bonded to a nitrogen group which is bonded to one or more alkyl groups. In a further case, which is also an alkylamido as the term is defined herein, the carbonyl carbon atom is bonded to a nitrogen atom which is bonded to one or more aryl group instead of, or in addition to, the one or more alkyl group. In a further case, which is also an alkylamido as the term is defined herein, the carbonyl carbon atom is bonded to a nitrogen atom which is bonded to one or more alkenyl group instead of, or in addition to, the one or more alkyl and or/aryl group. In a further case, which is also an alkylamido as the term is defined herein, the carbonyl carbon atom is bonded to a nitrogen atom which is bonded to one or more alkynyl group instead of, or in addition to, the one or more alkyl, alkenyl and/or aryl group.

The term “carboxy” refers to a group containing a carbonyl moiety wherein the group is bonded via the carbonyl carbon atom. The carbonyl carbon atom is also bonded to a hydroxy group or oxygen anion so as to result in a carboxylic acid or carboxylate. Carboxy also includes both the protonated form of the carboxylic acid and the salt form. For example, carboxy can be understood as COOH or CO₂H.

The term “alkylthio” refers to a sulfur atom connected to an alkyl, alkenyl, or alkynyl group as defined herein.

The term “arylthio” refers to a sulfur atom connected to an aryl group as defined herein.

The term “alkylsulfonyl” refers to a sulfonyl group connected to an alkyl, alkenyl, or alkynyl group as defined herein.

The term “alkylsulfinyl” refers to a sulfinyl group connected to an alkyl, alkenyl, or alkynyl group as defined herein.

The term “dialkylaminosulfonyl” refers to a sulfonyl group connected to a nitrogen further connected to two alkyl groups, as defined herein, and which can optionally be linked together to form a ring with the nitrogen. This term also includes the group where the nitrogen is further connected to one or two alkenyl groups in place of the alkyl groups.

The term “dialkylamino” refers to an amino group connected to two alkyl groups, as defined herein, and which can optionally be linked together to form a ring with the nitrogen. This term also includes the group where the nitrogen is further connected to one or two alkenyl groups in place of the alkyl groups.

The term “dialkylamido” refers to an amido group connected to two alkyl groups, as defined herein, and which can optionally be linked together to form a ring with the nitrogen. This term also includes the group where the nitrogen is further connected to one or two alkenyl groups in place of the alkyl groups.

The term “substituted” refers to a group that is substituted with one or more groups including, but not limited to, the following groups: halogen (e.g., F, Cl, Br, and I), R, OR, OC(O)N(R)₂, CN, NO, NO₂, ONO₂, azido, CF₃, OCF₃, (CH₂)₀₋₄B(OR)₂, methylenedioxy, ethylenedioxy, (C₃-C₂₀) heteroaryl, N(R)₂, Si(R)₃, SR, SOR, SO₂R, SO₂N(R)₂, SO₃, SO₃R, (CH₂)₀₋₄P(O)(OR)₂, (CH₂)₀₋₄O(CH₂)₀₋₄P(O)(OR)₂, C(O)R, C(O)C(O)R, C(O)CH₂C(O)R, C(S)R, C(O)OR, OC(O)R, C(O)N(R)₂, C(O)N(R)OH, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O) N(OR)R, or C(═NOR) R wherein R can be hydrogen, (C₁-C₂₀)alkyl or (C₆-C₂₀) aryl. Substituted also includes a group that is substituted with one or more groups including, but not limited to, the following groups: fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. Where there are two or more adjacent substituents, the substituents can be linked to form a carbocyclic or heterocyclic ring. Such adjacent groups can have a vicinal or germinal relationship, or they can be adjacent on a ring in, e.g., an ortho-arrangement. Each instance of substituted is understood to be independent. For example, a substituted aryl can be substituted with bromo and a substituted heterocycle on the same compound can be substituted with alkyl. It is envisaged that a substituted group can be substituted with one or more non-fluoro groups. As another example, a substituted group can be substituted with one or more non-cyano groups. As another example, a substituted group can be substituted with one or more groups other than haloalkyl. As yet another example, a substituted group can be substituted with one or more groups other than tert-butyl. As yet a further example, a substituted group can be substituted with one or more groups other than trifluoromethyl. As yet even further examples, a substituted group can be substituted with one or more groups other than nitro, other than methyl, other than methoxymethyl, other than dialkylaminosulfonyl, other than bromo, other than chloro, other than amido, other than halo, other than benzodioxepinyl, other than polycyclic heterocyclyl, other than polycyclic substituted aryl, other than methoxycarbonyl, other than alkoxycarbonyl, other than thiophenyl, or other than nitrophenyl, or groups meeting a combination of such descriptions. Further, substituted is also understood to include fluoro, cyano, haloalkyl, tert-butyl, trifluoromethyl, nitro, methyl, methoxymethyl, dialkylaminosulfonyl, bromo, chloro, amido, halo, benzodioxepinyl, polycyclic heterocyclyl, polycyclic substituted aryl, methoxycarbonyl, alkoxycarbonyl, thiophenyl, and nitrophenyl groups.

The terms “salts” and “pharmaceutically acceptable salts” refer to derivatives of the compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound, which contains a basic or acidic moiety, by conventional chemical methods. In some instances, such salts can be prepared by reacting the free acid or base forms of these compounds with a stoichiometric (or larger) amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

The term “solvate” means a compound, or a salt thereof, that further includes a stoichiometric or non-stoichiometric amount of solvent bound by non-covalent intermolecular forces. Where the solvent is water, the solvate is a hydrate.

The term “prodrug” means a derivative of a compound that can hydrolyze, oxidize, or otherwise react under biological conditions (in vitro or in vivo) to provide an active compound, particularly a compound of the invention. Examples of prodrugs include, but are not limited to, derivatives and metabolites of a compound of the invention that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues.

Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

The term “subject” or “patient” refers to any organism to which a composition described herein can be administered, e.g., for experimental, diagnostic, prophylactic and/or therapeutic purposes. Subject refers to a mammal receiving the compositions disclosed herein or subject to disclosed methods. It is understood and herein contemplated that “mammal” includes, but is not limited to, humans, non-human primates, cows, horses, dogs, cats, mice, rats, rabbits, and guinea pigs.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

The terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading can occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

Those skilled in the art will appreciate that many modifications to the embodiments described herein are possible without departing from the spirit and scope of the present disclosure. Thus, the description is not intended and should not be construed to be limited to the examples given but should be granted the full breadth of protection afforded by the appended claims and equivalents thereto. In addition, it is possible to use some of the features of the present disclosure without the corresponding use of other features. Accordingly, the foregoing description of or illustrative embodiments is provided for the purpose of illustrating the principles of the present disclosure and not in limitation thereof and can include modification thereto and permutations thereof.

The disclosure relates to, among other things, the following enumerated Embodiments, which listing does not represent an order of importance:

Embodiment 1. A compound of the formula (I):

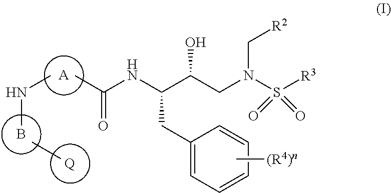

or a pharmaceutically acceptable salt thereof, wherein:
A is aryl;
B is heterocyclyl;
Q is alkyl or heterocyclyl;
R² is alkyl;
R³ is aryl, benzthiazole, benzoxazole, benzofuranyl or indolyl;
each R⁴ is, independently, alkyl, alkoxy, aryl, heterocyclyl, halo, hydroxy or amino; and
n is an integer from 0 to 3.

Embodiment 2. The compound of Embodiment 1, or a pharmaceutically acceptable salt thereof, wherein A is a monocyclic aryl group, which is optionally substituted.

Embodiment 3. The compound of Embodiment 2, or a pharmaceutically acceptable salt thereof, wherein A is substituted with an alkyl group.

Embodiment 4. The compound of Embodiment 1, or a pharmaceutically acceptable salt thereof, wherein A is phenyl.

Embodiment 5. The compound of Embodiment 1, or a pharmaceutically acceptable salt thereof, wherein B is a five- or six-membered monocyclic heterocyclyl group.

Embodiment 6. The compound of Embodiment 5, or a pharmaceutically acceptable salt thereof, wherein the monocyclic heterocyclic group has the formula:

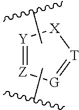

wherein X, Y, Z, G, and T are each, independently, C, CR⁵, N, NR⁶, S or O, wherein each R⁵ is independently H, alkyl or amino; and each R⁶ is independently H or alkyl.

Embodiment 7. The compound of Embodiment 6, or a pharmaceutically acceptable salt thereof, wherein X is S and G is N.

Embodiment 8. The compound of Embodiment 7, or a pharmaceutically acceptable salt thereof, wherein Z is CR⁵.

Embodiment 9. The compound of Embodiment 8, or a pharmaceutically acceptable salt thereof, wherein each R⁵ is independently H or (C₁-C₃)alkyl.

Embodiment 10. The compound of Embodiment 8, or a pharmaceutically acceptable salt thereof, wherein each R⁵ is H.

Embodiment 11. The compound of Embodiment 5, or a pharmaceutically acceptable salt thereof, wherein the monocyclic heterocyclic group has the formula:

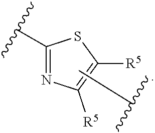

Embodiment 12. The compound of Embodiment 11, or a pharmaceutically acceptable salt thereof, wherein each R⁵ is H.

Embodiment 13. The compound of Embodiment 5, or a pharmaceutically acceptable salt thereof, wherein the monocyclic heterocyclic group has the formula:

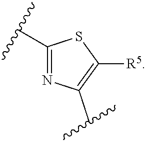

Embodiment 14. The compound of Embodiment 13, wherein the monocyclic heterocyclic group is attached to A and Q as shown:

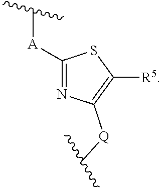

Embodiment 15. The compound of Embodiment 13 or 14, or a pharmaceutically acceptable salt thereof, wherein R⁵ is H.

Embodiment 16. The compound of Embodiment 5, or a pharmaceutically acceptable salt thereof, wherein the six-membered monocyclic heterocyclyl group has the formula:

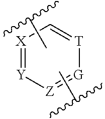

wherein X, Y, Z, G, and T are each, independently, C, CR⁵ or N, wherein each R⁵ is independently H, alkyl or amino.

Embodiment 17. The compound of Embodiment 16, or a pharmaceutically acceptable salt thereof, wherein at least one of X, Y, Z, G, and T is N.

Embodiment 18. The compound of Embodiment 16, or a pharmaceutically acceptable salt thereof, wherein at least two of X, Y, Z, G, and T is N.

Embodiment 19. The compound of Embodiment 16, or a pharmaceutically acceptable salt thereof, wherein if X, Y, Z, G, and T are CR⁵, each R⁵ is independently H or (C₁-C₃)alkyl.

Embodiment 20. The compound of Embodiment 19, or a pharmaceutically acceptable salt thereof, wherein each R⁵ is H.

Embodiment 21. The compound of Embodiment 16, or a pharmaceutically acceptable salt thereof, wherein the six-membered monocyclic heterocyclyl group has the formula:

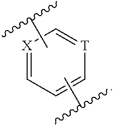

Embodiment 22. The compound of Embodiment 21, or a pharmaceutically acceptable salt thereof, wherein the six-membered monocyclic heterocyclyl group has the formula:

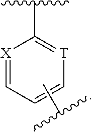

Embodiment 23. The compound of Embodiment 21 or 22, or a pharmaceutically acceptable salt thereof, wherein at least one of X and T is N.

Embodiment 24. The compound of Embodiment 23, or a pharmaceutically acceptable salt thereof, wherein X and T are N.

Embodiment 25. The compound of Embodiment 24, or a pharmaceutically acceptable salt thereof, wherein B is of the formula:

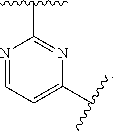 [SMILES: *c1ccnc(*)n1]

Embodiment 26. The compound of Embodiment 5, or a pharmaceutically acceptable salt thereof, wherein B is attached to A and Q as shown:

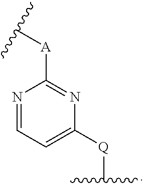

Embodiment 27. The compound of Embodiment 1, or a pharmaceutically acceptable salt thereof, wherein Q is a six-membered monocyclic heterocyclyl group.

Embodiment 28. The compound of Embodiment 27, or a pharmaceutically acceptable salt thereof, wherein the six-membered monocyclic heterocyclyl group has the formula:

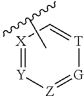

wherein X, Y, Z, G, and T are each, independently, C, CR⁵ or N, wherein each R⁵ is independently H, alkyl or amino.

Embodiment 29. The compound of Embodiment 28, or a pharmaceutically acceptable salt thereof, wherein at least one of X, Y, Z, G, and T is N.

Embodiment 30. The compound of Embodiment 29, or a pharmaceutically acceptable salt thereof, wherein at least two of X, Y, Z, G, and T is N.

Embodiment 31. The compound of Embodiment 28, or a pharmaceutically acceptable salt thereof, wherein if X, Y, Z, G, and T are CR⁵, each R⁵ is independently H or (C₁-C₃)alky.

Embodiment 32. The compound of Embodiment 31, or a pharmaceutically acceptable salt thereof, wherein each R⁵ is H.

Embodiment 33. The compound of Embodiment 1, or a pharmaceutically acceptable salt thereof, wherein Q is of the formula:

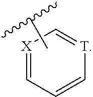

Embodiment 34. The compound of Embodiment 1, or a pharmaceutically acceptable salt thereof, wherein Q is of the formula:

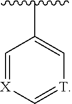

Embodiment 35. The compound of Embodiment 33 or 34, or a pharmaceutically acceptable salt thereof, wherein at least one of X and T is N.

Embodiment 36. The compound of Embodiment 33 or 34, or a pharmaceutically acceptable salt thereof, wherein X is N and T is CR⁵.

Embodiment 37. The compound of Embodiment 1, or a pharmaceutically acceptable salt thereof, wherein Q is of the formula:

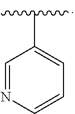 [SMILES: *c1cccnc1]

Embodiment 38. The compound of Embodiment 1, or a pharmaceutically acceptable salt thereof, wherein Q is alkyl, which is optionally substituted.

Embodiment 39. The compound of Embodiment 38, or a pharmaceutically acceptable salt thereof, wherein Q is (C₁-C₃)alkyl, which is optionally substituted.

Embodiment 40. The compound of Embodiment 38, or a pharmaceutically acceptable salt thereof, wherein Q is substituted with alkyl, halo, alkoxy, amino or aryl.

Embodiment 41. The compound of Embodiment 38, or a pharmaceutically acceptable salt thereof, wherein Q is haloalkyl or alkoxyalkyl.

Embodiment 42. The compound of Embodiment 38, or a pharmaceutically acceptable salt thereof, wherein Q is mono-, di- or tri-fluoroalkyl.

Embodiment 43. The compound of Embodiment 38, or a pharmaceutically acceptable salt thereof, wherein Q is (C₁-C₃)alkyl-O—(C₁-C₃)alkyl.

Embodiment 44. The compound of Embodiment 38, or a pharmaceutically acceptable salt thereof, wherein Q is-CH₂OCH₃.

Embodiment 45. The compound of Embodiment 1, or a pharmaceutically acceptable salt thereof, wherein R³ is aryl.

Embodiment 46. The compound of Embodiment 1, or a pharmaceutically acceptable salt thereof, wherein R³ is:

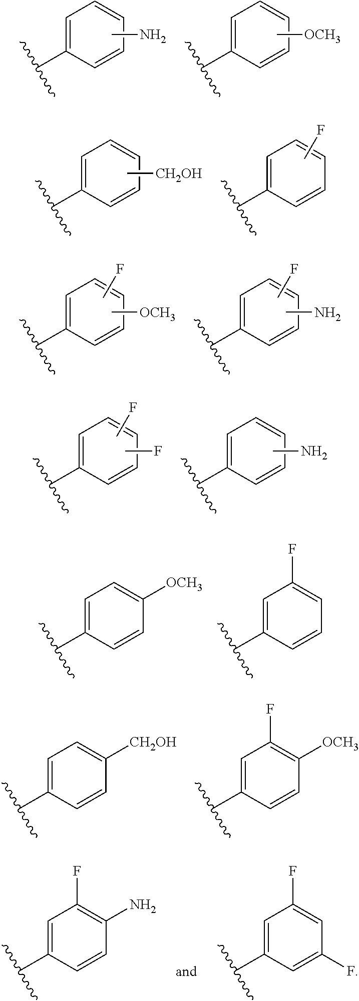

Embodiment 47. The compound of Embodiment 1, or a pharmaceutically acceptable salt thereof, wherein R² is (C₁-C₆)alkyl.

Embodiment 48. The compound of Embodiment 47, or a pharmaceutically acceptable salt thereof, wherein R² is (C₁-C₃)alkyl.

Embodiment 49. The compound of Embodiment 48, or a pharmaceutically acceptable salt thereof, wherein R² is (C₃)alkyl.

Embodiment 50. The compound of Embodiment 1, or a pharmaceutically acceptable salt thereof, wherein the compound has the formula:

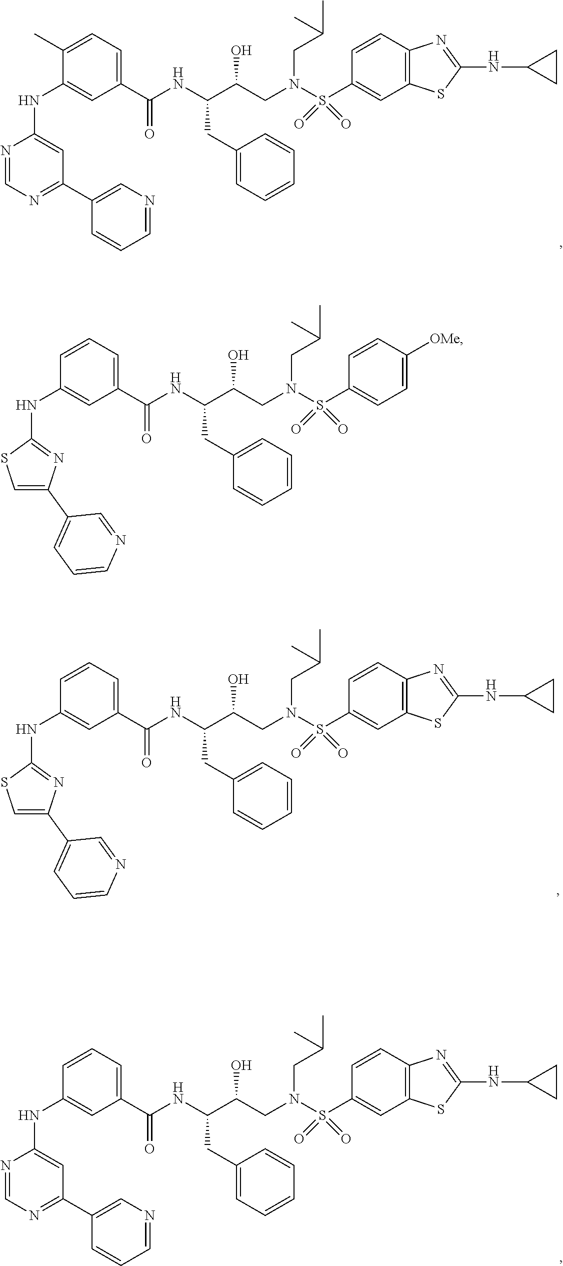 [SMILES: Cc1ccc(C(=O)N[C@@H](Cc2ccccc2)[C@H](O)CN(CC(C)C)S(=O)(=O)c2ccc3nc(NC4CC4)sc3c2)cc1Nc1cc(-c2cccnc2)ncn1 ; COc1ccc(S(=O)(=O)N(CC(C)C)C[C@@H](O)[C@H](Cc2ccccc2)NC(=O)c2cccc(Nc3nc(-c4cccnc4)cs3)c2)cc1 ; CC(C)CN(C[C@@H](O)[C@H](Cc1ccccc1)NC(=O)c1cccc(Nc2nc(-c3cccnc3)cs2)c1)S(=O)(=O)c1ccc2nc(NC3CC3)sc2c1 ; CC(C)CN(C[C@@H](O)[C@H](Cc1ccccc1)NC(=O)c1cccc(Nc2cc(-c3cccnc3)ncn2)c1)S(=O)(=O)c1ccc2nc(NC3CC3)sc2c1]

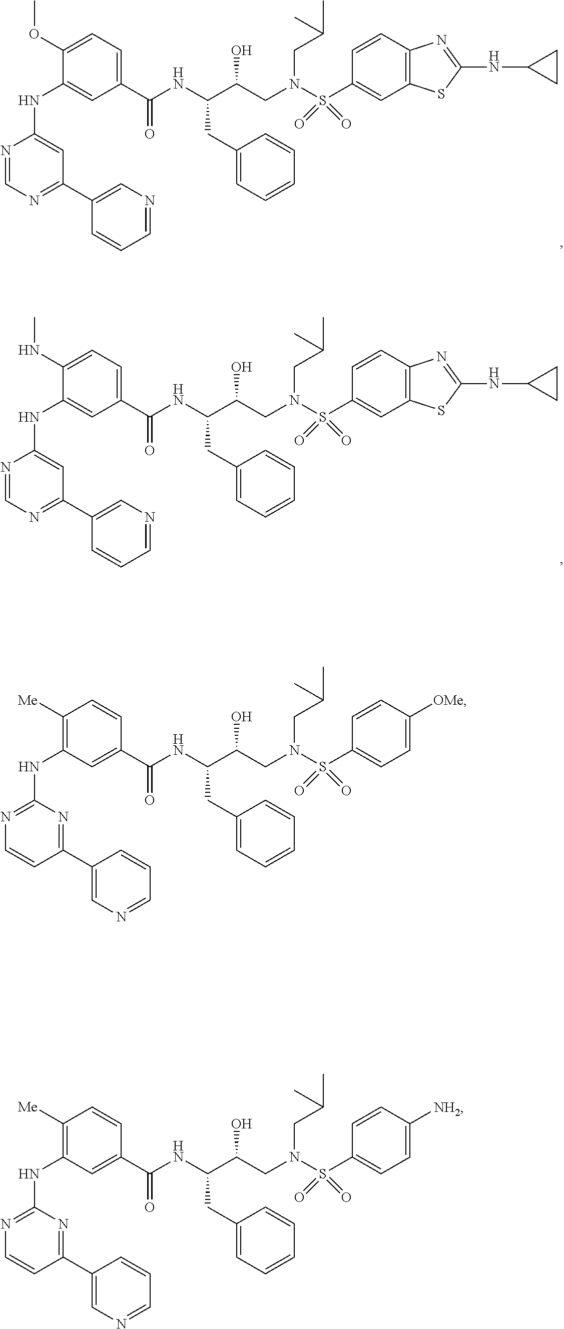 [SMILES: COc1ccc(C(=O)N[C@@H](Cc2ccccc2)[C@H](O)CN(CC(C)C)S(=O)(=O)c2ccc3nc(NC4CC4)sc3c2)cc1Nc1cc(-c2cccnc2)ncn1 ; CNc1ccc(C(=O)N[C@@H](Cc2ccccc2)[C@H](O)CN(CC(C)C)S(=O)(=O)c2ccc3nc(NC4CC4)sc3c2)cc1Nc1cc(-c2cccnc2)ncn1 ; COc1ccc(S(=O)(=O)N(CC(C)C)C[C@@H](O)[C@H](Cc2ccccc2)NC(=O)c2ccc(C)c(Nc3nccc(-c4cccnc4)n3)c2)cc1 ; Cc1ccc(C(=O)N[C@@H](Cc2ccccc2)[C@H](O)CN(CC(C)C)S(=O)(=O)c2ccc(N)cc2)cc1Nc1nccc(-c2cccnc2)n1]

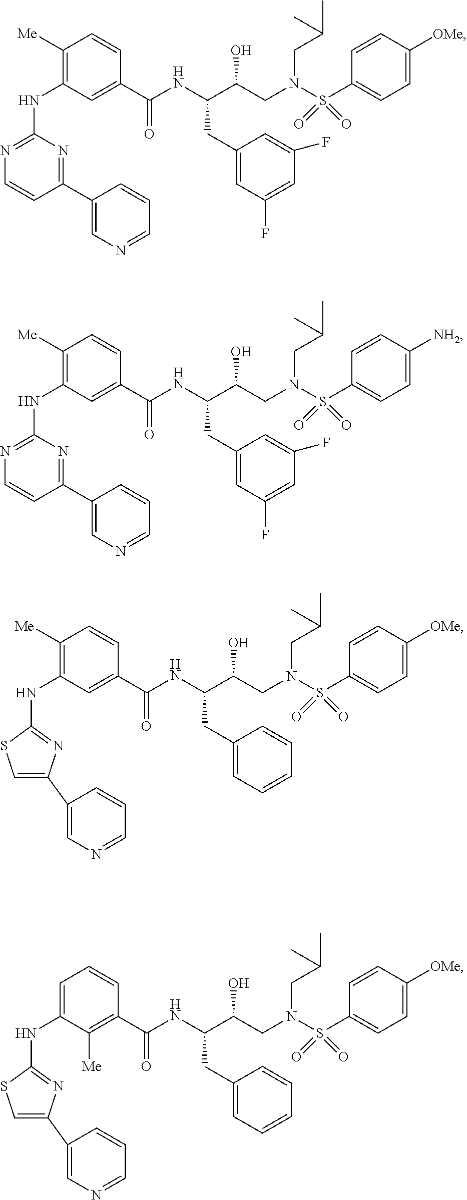 [SMILES: COc1ccc(S(=O)(=O)N(CC(C)C)C[C@@H](O)[C@H](Cc2cc(F)cc(F)c2)NC(=O)c2ccc(C)c(Nc3nccc(-c4cccnc4)n3)c2)cc1 ; Cc1ccc(C(=O)N[C@@H](Cc2cc(F)cc(F)c2)[C@H](O)CN(CC(C)C)S(=O)(=O)c2ccc(N)cc2)cc1Nc1nccc(-c2cccnc2)n1 ; COc1ccc(S(=O)(=O)N(CC(C)C)C[C@@H](O)[C@H](Cc2ccccc2)NC(=O)c2ccc(C)c(Nc3nc(-c4cccnc4)cs3)c2)cc1 ; COc1ccc(S(=O)(=O)N(CC(C)C)C[C@@H](O)[C@H](Cc2ccccc2)NC(=O)c2cccc(Nc3nc(-c4cccnc4)cs3)c2C)cc1]

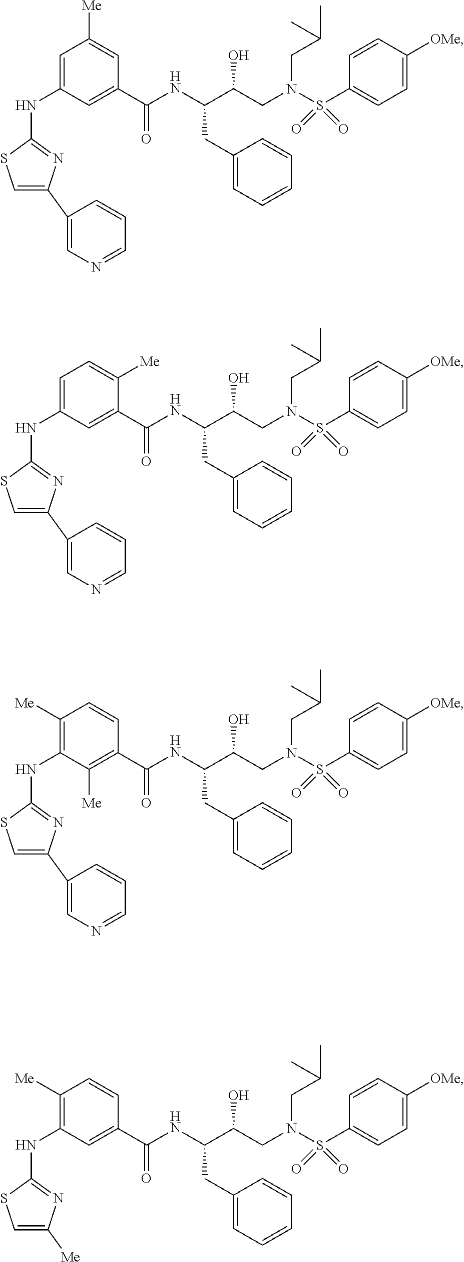 [SMILES: COc1ccc(S(=O)(=O)N(CC(C)C)C[C@@H](O)[C@H](Cc2ccccc2)NC(=O)c2cc(C)cc(Nc3nc(-c4cccnc4)cs3)c2)cc1 ; COc1ccc(S(=O)(=O)N(CC(C)C)C[C@@H](O)[C@H](Cc2ccccc2)NC(=O)c2cc(Nc3nc(-c4cccnc4)cs3)ccc2C)cc1 ; COc1ccc(S(=O)(=O)N(CC(C)C)C[C@@H](O)[C@H](Cc2ccccc2)NC(=O)c2ccc(C)c(Nc3nc(-c4cccnc4)cs3)c2C)cc1 ; COc1ccc(S(=O)(=O)N(CC(C)C)C[C@@H](O)[C@H](Cc2ccccc2)NC(=O)c2ccc(C)c(Nc3nc(C)cs3)c2)cc1]

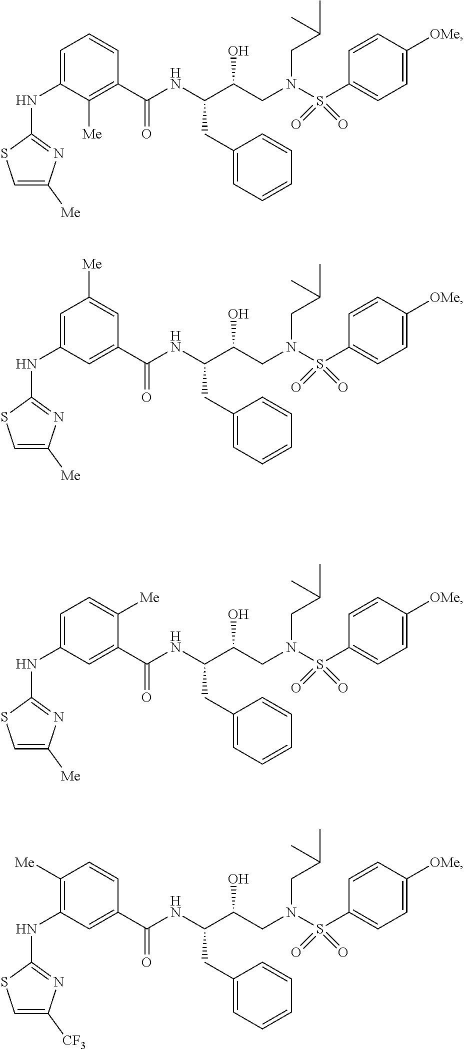 [SMILES: COc1ccc(S(=O)(=O)N(CC(C)C)C[C@@H](O)[C@H](Cc2ccccc2)NC(=O)c2cccc(Nc3nc(C)cs3)c2C)cc1 ; COc1ccc(S(=O)(=O)N(CC(C)C)C[C@@H](O)[C@H](Cc2ccccc2)NC(=O)c2cc(C)cc(Nc3nc(C)cs3)c2)cc1 ; COc1ccc(S(=O)(=O)N(CC(C)C)C[C@@H](O)[C@H](Cc2ccccc2)NC(=O)c2cc(Nc3nc(C)cs3)ccc2C)cc1 ; COc1ccc(S(=O)(=O)N(CC(C)C)C[C@@H](O)[C@H](Cc2ccccc2)NC(=O)c2ccc(C)c(Nc3nc(C(F)(F)F)cs3)c2)cc1]

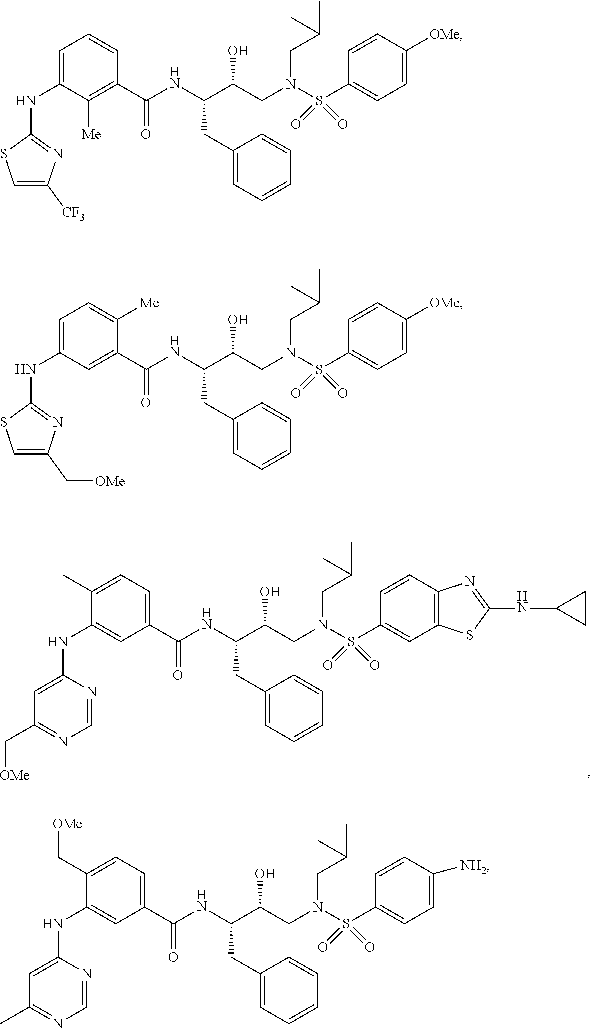 [SMILES: COc1ccc(S(=O)(=O)N(CC(C)C)C[C@@H](O)[C@H](Cc2ccccc2)NC(=O)c2cccc(Nc3nc(C(F)(F)F)cs3)c2C)cc1 ; COCc1csc(Nc2ccc(C)c(C(=O)N[C@@H](Cc3ccccc3)[C@H](O)CN(CC(C)C)S(=O)(=O)c3ccc(OC)cc3)c2)n1 ; COCc1cc(Nc2cc(C(=O)N[C@@H](Cc3ccccc3)[C@H](O)CN(CC(C)C)S(=O)(=O)c3ccc4nc(NC5CC5)sc4c3)ccc2C)ncn1 ; COCc1ccc(C(=O)N[C@@H](Cc2ccccc2)[C@H](O)CN(CC(C)C)S(=O)(=O)c2ccc(N)cc2)cc1Nc1cc(C)ncn1]

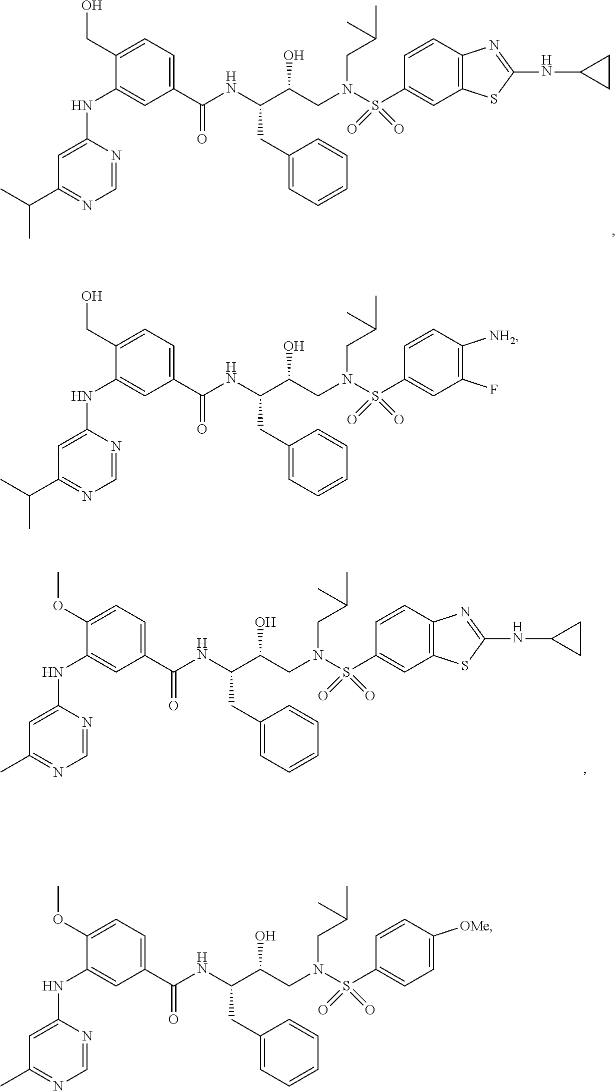 [SMILES: CC(C)CN(C[C@@H](O)[C@H](Cc1ccccc1)NC(=O)c1ccc(CO)c(Nc2cc(C(C)C)ncn2)c1)S(=O)(=O)c1ccc2nc(NC3CC3)sc2c1 ; CC(C)CN(C[C@@H](O)[C@H](Cc1ccccc1)NC(=O)c1ccc(CO)c(Nc2cc(C(C)C)ncn2)c1)S(=O)(=O)c1ccc(N)c(F)c1 ; COc1ccc(C(=O)N[C@@H](Cc2ccccc2)[C@H](O)CN(CC(C)C)S(=O)(=O)c2ccc3nc(NC4CC4)sc3c2)cc1Nc1cc(C)ncn1 ; COc1ccc(S(=O)(=O)N(CC(C)C)C[C@@H](O)[C@H](Cc2ccccc2)NC(=O)c2ccc(OC)c(Nc3cc(C)ncn3)c2)cc1]

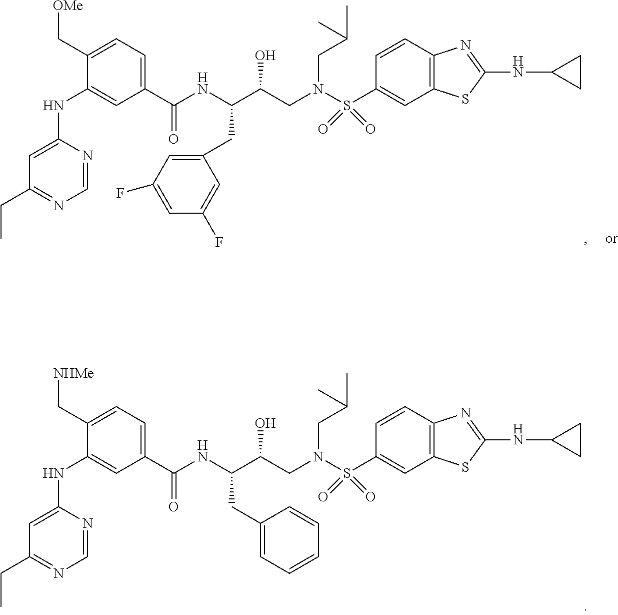 [SMILES: CCc1cc(Nc2cc(C(=O)N[C@@H](Cc3cc(F)cc(F)c3)[C@H](O)CN(CC(C)C)S(=O)(=O)c3ccc4nc(NC5CC5)sc4c3)ccc2COC)ncn1 ; CCc1cc(Nc2cc(C(=O)N[C@@H](Cc3ccccc3)[C@H](O)CN(CC(C)C)S(=O)(=O)c3ccc4nc(NC5CC5)sc4c3)ccc2CNC)ncn1]

Embodiment 51. A pharmaceutical composition comprising the compound of any of Embodiments 1 to 50, or a pharmaceutically acceptable salt or hydrate thereof, and one or more pharmaceutically acceptable excipients.

Embodiment 52. A method for treating an HIV infection or AIDS, the method comprising administering a therapeutically effective amount of one or more compounds of any of Embodiments 1 to 50, or a pharmaceutical composition comprising same, or a pharmaceutically acceptable salt thereof, and one or more pharmaceutically acceptable excipients, to a subject in need thereof.

Embodiment 53. The compound of any of Embodiments 1 to 50, or a pharmaceutically acceptable salt thereof, wherein the compound has an HIV-1 protease inhibition constant (Kᵢ) of about 1 μM to about 100 nM.

Embodiment 54. The compound of any of Embodiments 1 to 50, or a pharmaceutically acceptable salt thereof, wherein the compound has an antiviral activity in vitro against a wild-type laboratory strain, HIV-1_{LAI}, with a half-maximal inhibitory concentration (IC₅₀) of about 1 nM to about 600 nM.

Embodiment 55. The compound of any of Embodiments 1 to 50, or a pharmaceutically acceptable salt thereof, wherein the compound has an antiviral activity in vitro against a darunavir-resistant HIV-1 variant with an IC₅₀ of about 1 nM to about 600 nM.

Embodiment 56. The compound of any of Embodiments 1 to 50, or a pharmaceutically acceptable salt thereof, wherein the compound has an antiviral activity in vitro against a wild-type laboratory strain, HIV-1_{LAI}, and an antiviral activity in vitro against a darunavir-resistant HIV-1 variant with an IC₅₀ of about 1 nM to about 600 nM.

Embodiment 57. The compound of Embodiment 56, or a pharmaceutically acceptable salt thereof, wherein the darunavir-resistant HIV-1 variant is at least one of DRV_{R}P20, DRV_{R}P30, and DRV_{R}P51.

### EXAMPLES

The disclosure can be better understood by reference to the following examples which are offered by way of illustration. The disclosure is not limited to the examples given herein.

### Introduction

A series of HIV-1 protease inhibitors that incorporated substituted isophthalamide derivatives as the P2-ligands were reported recently. A representative example inhibitor 6 is shown in Scheme 2. These inhibitors were designed by taking advantage of the large hydrophobic pocket in the HIV-1 protease S1-S2 subsites. While the P2-ligands resemble 3-hydroxy-2-methyl benzamide inherent to nelfinavir 7, an FDA approved drug, X-ray structural studies of inhibitor-bound HIV-1 protease revealed that the 3-hydroxy benzylamide carbonyl involved in hydrogen bonding interaction with the backbone amide NH of Asp29. However, the oxazolylmethyl segment is mostly solvent exposed and does not participate in any polar interactions in the active site. Based upon this structural precedence, pyridyl pyrimidine and various alkyl, arylthiazolyl hetereocycles such as 8 and 9 in combination with hydroxyethylamine sulfonamide isosteres were investigated. Preliminary models of pyridyl pyrimidine derivatives, based upon the X-ray structure of nelfinavir-bound HIV-1 protease, revealed that pyridyl pyrimidine functionalities can form hydrogen bonds with backbone amide NHs in the S2 subsite. Also, appropriately functionalized alkyl or aryl substituents can effectively fill in the hydrophobic pocket in the S2-S3 subsites. There is no previous report of the use of pyridyl pyrimidine derived P2 ligands in the design of HIV-1 protease inhibitors.

The results discussed in Examples 1 and 2 relate to the design and synthesis of a new class of HIV-1 protease inhibitors incorporating the pyridyl pymiridine nilotinib subunit and aryl thiazole derivatives of dasatinib as the P2 ligands. The results in Examples 3 and 4 relate to the activity profiles of methyl thiazole derivatives, as well as the effect of methyl substitution on the benzamide ring. Various alkyl and aryl thiazoles were conveniently prepared using Hantzsch thiazole synthesis. Several compounds exhibited very potent enzyme inhibitory activity in low nanomolar to subnanomolar level. A number of compounds also displayed low nanomolar antiviral activity. In general, pyridylpymiridine benzamide derivatives provided the most promising results.

With reference to Tables 1 and 2 herein, compound 5a with a 4-methoxysulfonamide as the P2′-ligand turned out be the most potent compound, which exhibited an enzyme inhibitory K; of 28 PM and antiviral activity of 154 nM. Compound 5b with a 4-aminosulfonamide as the P2′-ligand displayed potent antiviral activity with IC₅₀ value of 66 nM. Incorporation of lipophilic 1,3-difluorophenyl P1-ligand did not improve antiviral activity. Among thiazole derived inhibitors, compound 5 h with a pyridyl thiazole as the P2 ligand showed the best result exhibiting an HIV-1 protease inhibitory Kᵢ of 8.7 nM and antiviral activity of 580 nM. Methyl thiazole-derived compounds showed low nanomolar HIV-1 protease activity but no appreciable antiviral activity.

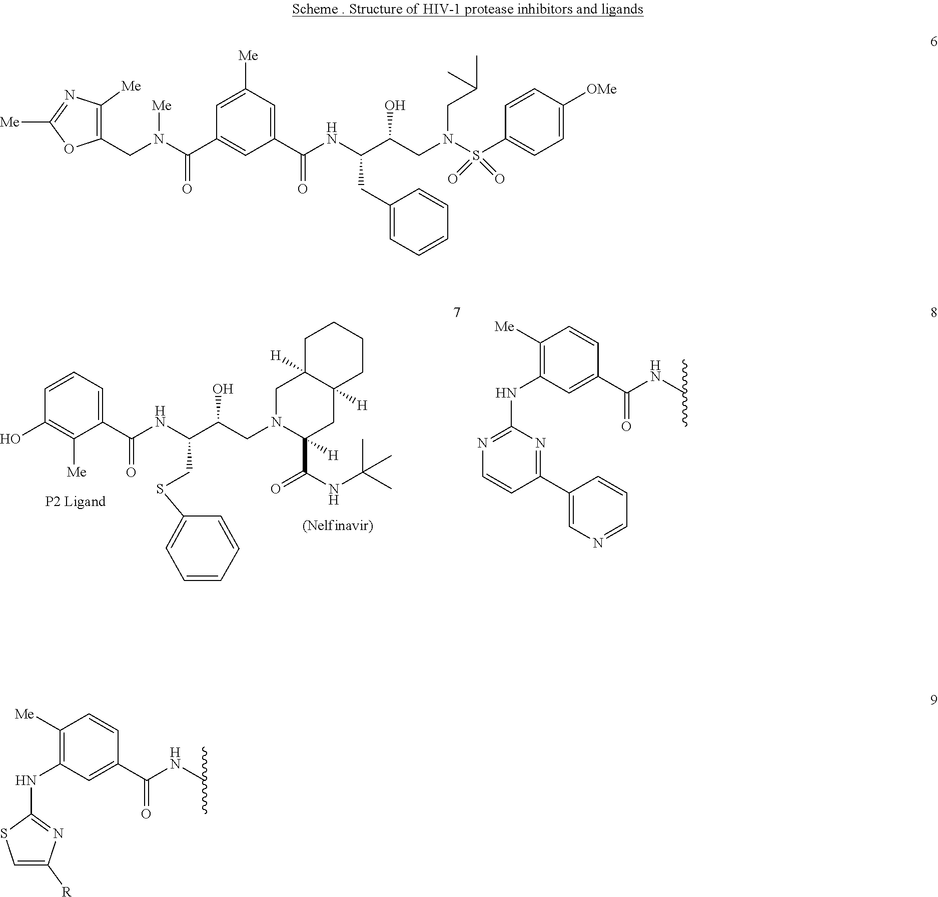

### Example 1: Synthesis of Nilotinib-Based HIV-1 Protease Inhibitors 5a-d

The synthesis of various nilotinib-based HIV-1 protease inhibitors 5a-d is shown in Scheme 3. Commercially available 4-methyl-3-[[4-(3-pyridinyl)-2-pyrimidinyl]amino]benzoic acid 10 was reacted with previously reported hydroxyethylene isosteres 11-14 in the presence of HATU and Et₃N in DMF at 23° C. for 24 h to afford the desired coupling products 5a-d in good yield (60-76%). The syntheses of various methyl-3-[[4-(3-pyridinyl)-2-thiazolyl]amino]benzoic acids and syntheses of inhibitors 5e-i are shown in Scheme 4. Hantzsch thiazole synthesis was utilized to obtain these desired pyridinyl-thiazolylamino benzoic acid derivatives. As shown, various methylsubstituted aminomethylbenzoates 15 were reacted with potassium thiocyante and acetyl chloride in acetone at 23° C. to 60° C. to provide thiourea derivatives 16. Exposure of 16 to potassium carbonate in methanol, followed by addition of α-bromo ketone 17 furnished pyridinylthiazole derivatives 18. Saponification of methyl esters 18 with 1 M LiOH in THE at 23° C. afforded ligand carboxylic acids 19a-e. Coupling of these ligand acids with hydroxyethylsulfonamide isosteric amine 11 provided inhibitors 5e-i in good yields.

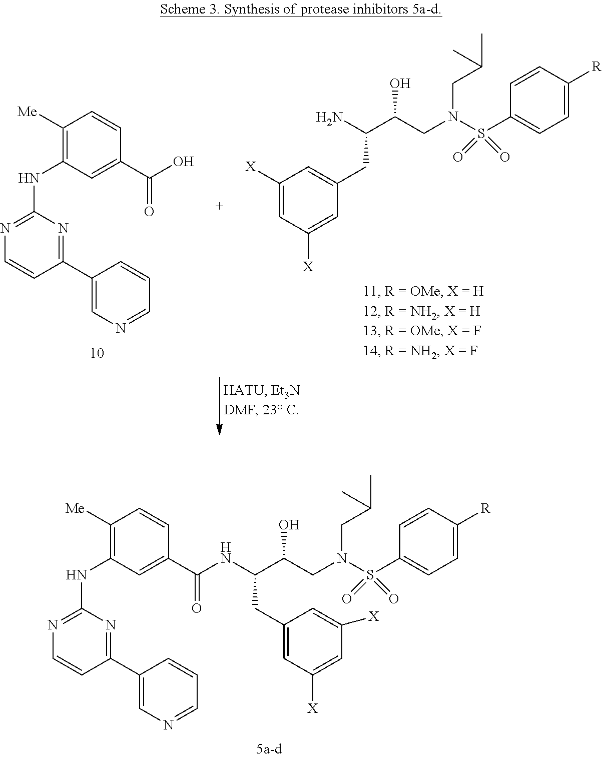

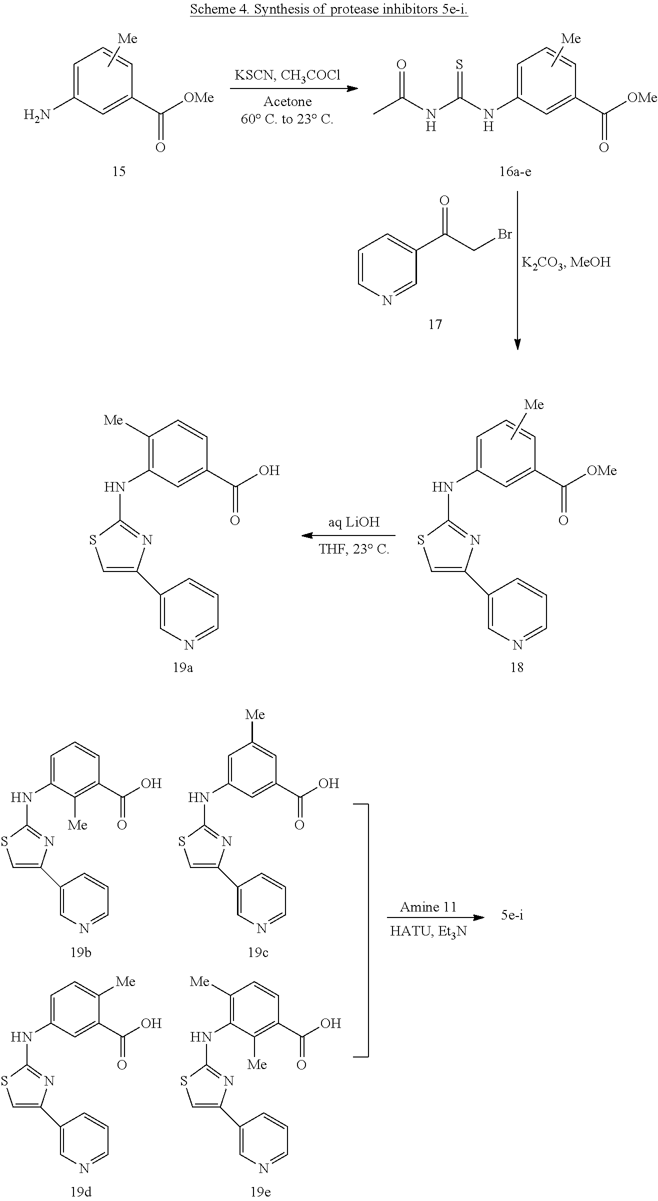

### Example 2: Synthesis of Various Methyl Substituted Thiazole Ligands and their Conversion to Inhibitors 5j-m

The synthesis of various methyl substituted thiazole ligands and their conversion to inhibitors 5j-m are shown in Scheme 5. Methyl thiazole derivatives 20a-d were synthesized utilizing an efficient one step procedure reported by Lagoja and Schantl. This procedure involves reaction of potassium thiocyanate with chloroacetone at 23° C. followed by addition of amino methyl esters 15a-d and heating to 60° C. in methanol for 6 h to provide 20a-d. The procedure resulted in good yields of these thiazole derivatives. However, yields are lower if the reactions are carried out longer than 6 h. Saponification of methyl esters 20 with 1 M LiOH in THF at 23° C. provided carboxylic acids 21a-d. Coupling of these ligand acids with amine 11 as described above provided inhibitors 5j-m in good yields.

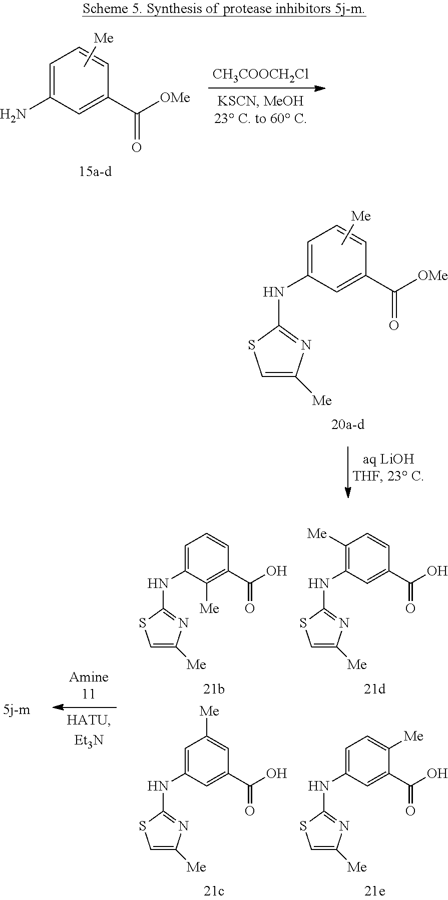

The synthesis of trifluoromethyl and methoxymethyl substituted thiazole ligand acids and their conversion to inhibitors 5n-p are shown in Scheme 6. For the synthesis of trifluoromethyl substituted ligand acids, thiourea derivatives 16a and 16b were reacted with trifluoromethyl ketone 22 to provide thiazole ester derivatives 23a and 23b. Saponification of the methyl esters furnished ligand acids 24a and 24b. Coupling of these acids with amine 11 resulted in inhibitors 5m and 5n, respectively. Methoxymethyl substituted thiazole ligand acid was synthesized from thiourea derivative 16d and the known α-bromo ketone 25, which was prepared using the reported procedure. This bromo ketone derivative is known to undergo Favorskii rearrangement upon heating, standing, or in the presence of base to provide the undesired secondary α-bromide. Therefore, freshly prepared bromo ketone was used. The reaction of 25 with thiourea 16d was carried out with lower equivalents of potassium carbonate. The thiazole derivative 26 was obtained in low yield possibly due the extreme base sensitivity. Saponification of ester 26 followed by coupling of the resulting acid 27 with amine 11 afforded inhibitor 5p.

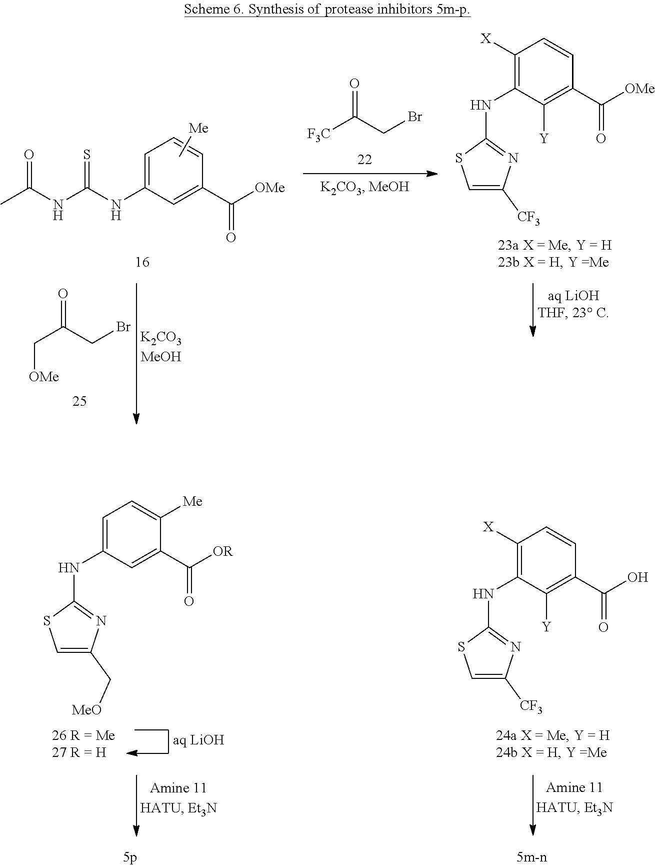

### Example 3: Study of Ability of the Nilotinib Subunit, Pyridinyl-Pyrimidinyl Amino Benzamides to Serve as P2 Ligands in the S2 Subsite of HIV-1 Protease

The ability of the nilotinib subunit, pyridinyl-pyrimidinyl amino benzamides to serve as P2 ligands in the S2 subsite of HIV-1 protease was investigated. The structure and activity of these inhibitors are shown in Table 1. The HIV-1 protease inhibitory activity of these compounds was evaluated in an enzyme-inhibitory assay reported by Toth and Marshall. As shown, compound 5a with a nilotinib pyridinyl-pyrimidinyl heterocycle as the P2 ligand and 4-methoxy sulfonamide as P2′-ligand exhibited very potent HIV-1 protease inhibitory activity with a Kᵢ of 0.028 nM. The corresponding 4-aminosulfonamide derivative 5b displayed nearly 5-fold reduction in inhibitory Kᵢ value (entry 2). We also determined the antiviral activity of these inhibitors in MT-2 human-T-lymphoid cells exposed to HIV_{LAI}. In this antiviral assay, compound 5a showed a potent antiviral IC₅₀ value of 154 nM. Interestingly, compound 5b showed a further improvement in antiviral activity compared to its 4-methoxy derivative, 5a. Compound 5c with 3,5-difluorophenylmethyl as the P1 ligand did not improve inhibitory activity and showed only a slight improvement in antiviral activity over compound 5a (entry 3). The corresponding 4-amino derivative 5d did not improve activity over compound 5b. Interestingly, compound 5d exhibited 3-fold reduction in antiviral activity over compound 5c (entries 3 and 4). We then examined a pyridyl thiazole heterocycle in place of the pyridyl pyrimidinyl scaffold in compounds 5a-d. Compound 5e, with a 4-methylbenzamide scaffold showed reduced inhibitory activity compared to its pyridylpyrimidine derivative 5a. This compound showed antiviral IC₅₀ value of 390 nM. The 2-methyl-3-aminothiozolyl derivative 5f displayed an enzyme inhibitory Kᵢ of 19 nM but did not exhibit any appreciable antiviral activity (entry 6). Compound 5g with a 3-aminothiazoloyl-5-methyl heterocycle did not improve any activity (entry 7). Interestingly, 3-aminothiazoloyl-6-methyl derivative 5 h showed an improvement in Kᵢ value (8.7 nM). Also, this compound showed an antiviral IC₅₀ value of 580 nM (entry 8). Compound 5i with a 2,4-dimethyl-3-aminothiazolyl derivative showed reduction in Kᵢ value, but maintained an antiviral IC₅₀ of 510 nM, comparable to derivative 5h (entries 8, 9).

### Example 4: Study of Effect of Alkyl Substitution on the 3-Thiazolyl Group as Well as Methyl Substitution on the Benzamide Scaffold

The effect of alkyl substitution on the 3-thiazolyl group as well as methyl substitution on the benzamide scaffold was investigated. The structure and activity of various derivatives are shown in Table 2. Compound 5j with a 3-methyl-thiazolylamino heterocycle showed a slight improvement in HIV-1 inhibitory activity (Kᵢ value 26 nM) over the corresponding 3-pyridine derivative 5e. However, it did not show any appreciable antiviral activity (entry 1). We have examined the effect of methyl substitution on the benzamide ring. Compounds 5k-m with 2-methyl, 5-methyl, and 6-methyl substitutions resulted in no improvement of activity over 4-methyl derivative 5j (entries 1-4). Also, substitution of a 2-trifluoromethyl group in compounds 5n and 50 did not improve any activity over their methyl derivatives (entries 5 and 6). Incorporation of a methoxy group on the 2-methyl derivative in compound 5p showed a slight improvement in inhibitory activity. This compound also exhibited antiviral activity IC₅₀ value of 630 nM (entry 7).

TABLE 1
Structure and activity of pyrimidine- and thiozole-derived protease
inhibitors

| Entry | Inhibitor | Kᵢ (nM) | IC₅₀ (nM) |
|---|---|---|---|
| 1. | 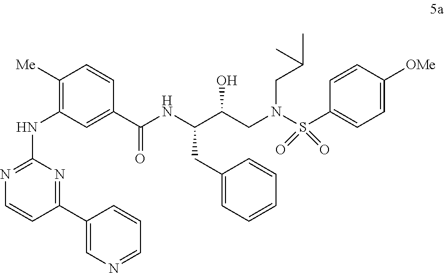 [SMILES: COc1ccc(S(=O)(=O)N(CC(C)C)C[C@@H](O)[C@H](Cc2ccccc2)NC(=O)c2ccc(C)c(Nc3nccc(-c4cccnc4)n3)c2)cc1] | 0.028 | 154 |
| | 5a | | |
| 2. | 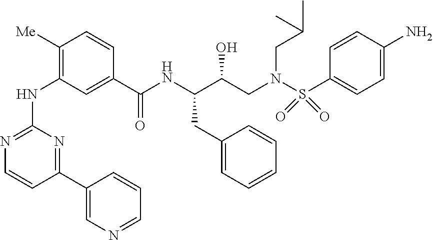 [SMILES: Cc1ccc(C(=O)N[C@@H](Cc2ccccc2)[C@H](O)CN(CC(C)C)S(=O)(=O)c2ccc(N)cc2)cc1Nc1nccc(-c2cccnc2)n1] | 0.17 | 66.8 |
| | 5b | | |
| 3. | 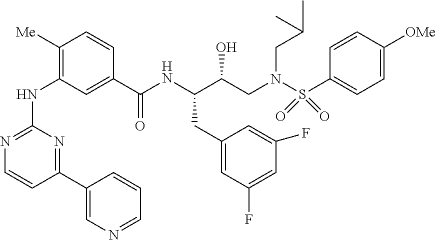 [SMILES: COc1ccc(S(=O)(=O)N(CC(C)C)C[C@@H](O)[C@H](Cc2cc(F)cc(F)c2)NC(=O)c2ccc(C)c(Nc3nccc(-c4cccnc4)n3)c2)cc1] | 0.58 | 124 |
| | 5c | | |
| 4. | 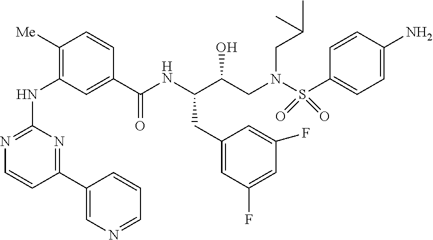 [SMILES: Cc1ccc(C(=O)N[C@@H](Cc2cc(F)cc(F)c2)[C@H](O)CN(CC(C)C)S(=O)(=O)c2ccc(N)cc2)cc1Nc1nccc(-c2cccnc2)n1] | 1.92 | 212 |
| | 5d | | |
| 5. | 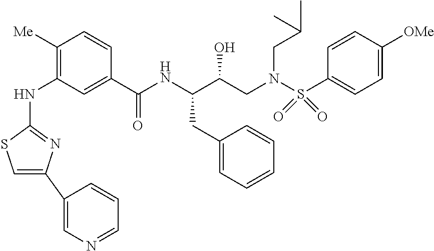 [SMILES: COc1ccc(S(=O)(=O)N(CC(C)C)C[C@@H](O)[C@H](Cc2ccccc2)NC(=O)c2ccc(C)c(Nc3nc(-c4cccnc4)cs3)c2)cc1] | 38.3 | 390 |
| | 5e | | |
| 6. | 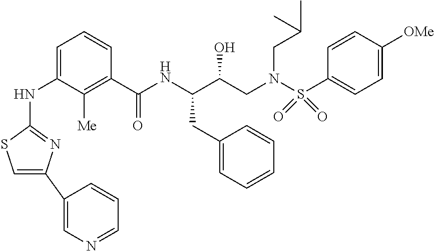 [SMILES: COc1ccc(S(=O)(=O)N(CC(C)C)C[C@@H](O)[C@H](Cc2ccccc2)NC(=O)c2cccc(Nc3nc(-c4cccnc4)cs3)c2C)cc1] | 19.3 | >1 |
| | 5f | | |
| 7. | 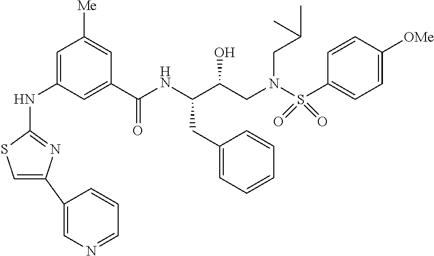 [SMILES: COc1ccc(S(=O)(=O)N(CC(C)C)C[C@@H](O)[C@H](Cc2ccccc2)NC(=O)c2cc(C)cc(Nc3nc(-c4cccnc4)cs3)c2)cc1] | 185.2 | >1 |
| | 5g | | |
| 8. | 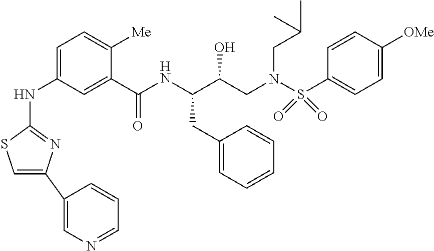 [SMILES: COc1ccc(S(=O)(=O)N(CC(C)C)C[C@@H](O)[C@H](Cc2ccccc2)NC(=O)c2cc(Nc3nc(-c4cccnc4)cs3)ccc2C)cc1] | 8.7 | 580 |
| | 5h | | |
| 9. | 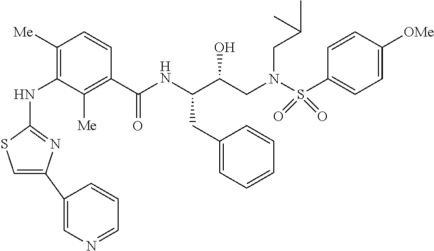 [SMILES: COc1ccc(S(=O)(=O)N(CC(C)C)C[C@@H](O)[C@H](Cc2ccccc2)NC(=O)c2ccc(C)c(Nc3nc(-c4cccnc4)cs3)c2C)cc1] | 122 | 510 |
| | 5i | | |
| 10. | 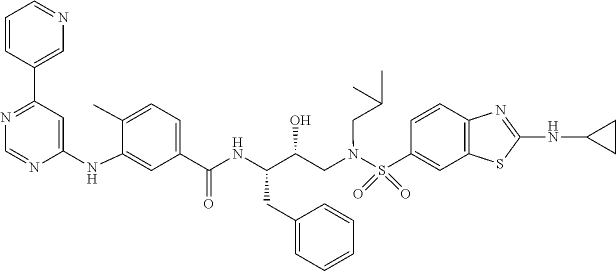 [SMILES: Cc1ccc(C(=O)N[C@@H](Cc2ccccc2)[C@H](O)CN(CC(C)C)S(=O)(=O)c2ccc3nc(NC4CC4)sc3c2)cc1Nc1cc(-c2cccnc2)ncn1] | Low nM | Low nM |
| 11. | 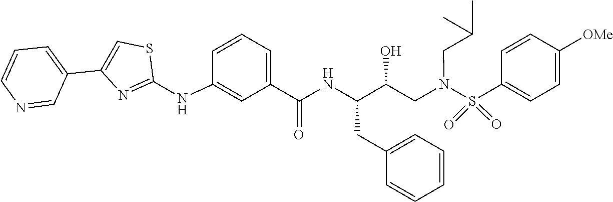 [SMILES: COc1ccc(S(=O)(=O)N(CC(C)C)C[C@@H](O)[C@H](Cc2ccccc2)NC(=O)c2cccc(Nc3nc(-c4cccnc4)cs3)c2)cc1] | Low nM | Low nM |
| 12. | 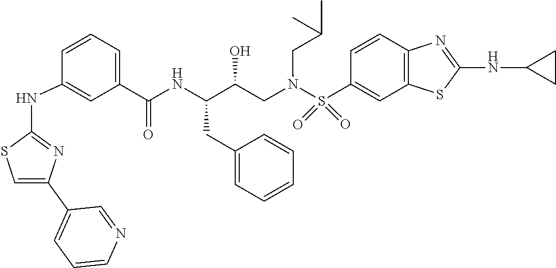 [SMILES: CC(C)CN(C[C@@H](O)[C@H](Cc1ccccc1)NC(=O)c1cccc(Nc2nc(-c3cccnc3)cs2)c1)S(=O)(=O)c1ccc2nc(NC3CC3)sc2c1] | Low nM | Low nM |

TABLE 2
Structure and activity of substituted thiazolyl-derived protease inhibitors

| Entry | Inhibitor | Kᵢ (nM) | IC₅₀ (nM) |
|---|---|---|---|
| 1. | 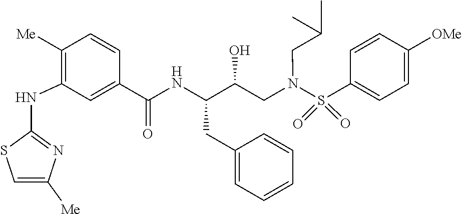 [SMILES: COc1ccc(S(=O)(=O)N(CC(C)C)C[C@@H](O)[C@H](Cc2ccccc2)NC(=O)c2ccc(C)c(Nc3nc(C)cs3)c2)cc1] | 26.5 | >1 |
| 2. | 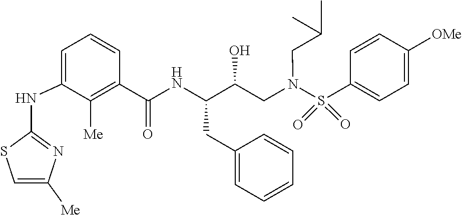 [SMILES: COc1ccc(S(=O)(=O)N(CC(C)C)C[C@@H](O)[C@H](Cc2ccccc2)NC(=O)c2cccc(Nc3nc(C)cs3)c2C)cc1] | 71.9 | >1 |
| 3. | 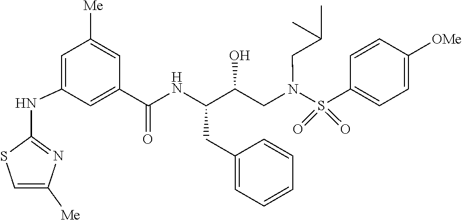 [SMILES: COc1ccc(S(=O)(=O)N(CC(C)C)C[C@@H](O)[C@H](Cc2ccccc2)NC(=O)c2cc(C)cc(Nc3nc(C)cs3)c2)cc1] | 80.0 | >1 |
| 4. | 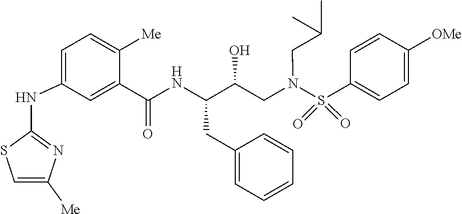 [SMILES: COc1ccc(S(=O)(=O)N(CC(C)C)C[C@@H](O)[C@H](Cc2ccccc2)NC(=O)c2cc(Nc3nc(C)cs3)ccc2C)cc1] | 75.9 | >1 |
| 5. | 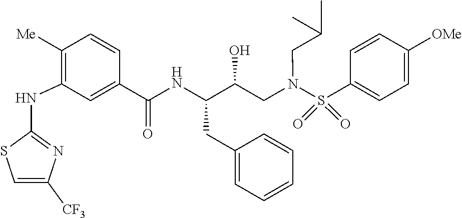 [SMILES: COc1ccc(S(=O)(=O)N(CC(C)C)C[C@@H](O)[C@H](Cc2ccccc2)NC(=O)c2ccc(C)c(Nc3nc(C(F)(F)F)cs3)c2)cc1] | 60.3 | >1 |
| 6. | 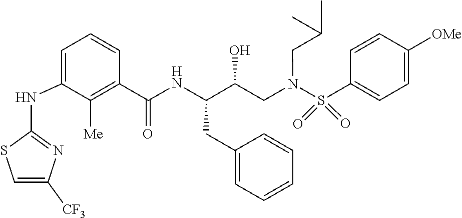 [SMILES: COc1ccc(S(=O)(=O)N(CC(C)C)C[C@@H](O)[C@H](Cc2ccccc2)NC(=O)c2cccc(Nc3nc(C(F)(F)F)cs3)c2C)cc1] | 72 | >1 |
| 7. | 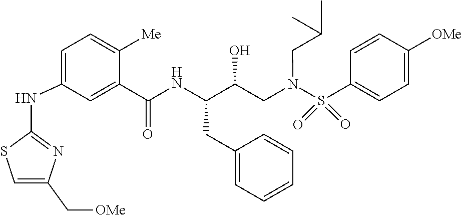 [SMILES: COCc1csc(Nc2ccc(C)c(C(=O)N[C@@H](Cc3ccccc3)[C@H](O)CN(CC(C)C)S(=O)(=O)c3ccc(OC)cc3)c2)n1] | 18.0 | 0.63 |

### Example 5: Detailed Syntheses of Compound Described Herein

### Experimental Methods

All moisture-sensitive reactions were carried out in oven-dried glassware under an argon atmosphere unless otherwise stated. Anhydrous solvents were obtained as follows: Tetrahydrofuran was distilled from sodium metal/benzophenone under argon. Dichloromethane was distilled from calcium hydride under argon. All other solvents were reagent grade. Column chromatography was performed using Silicycle Silia Flash F60 230-400 mesh silica gel. Thin layer chromatography was carried out using EMD Millipore TLC silica gel 60 F254 plates. ¹H NMR and ¹³C NMR spectra were recorded on a Bruker AV-III-400, Bruker DRX500, or Bruker AV-111-800. Low-resolution mass spectra were collected on an LCMS. High-resolution mass spectra were collected by the Purdue University Campus-Wide Mass Spectrometry Center. HPLC analysis and purification were done on an Agilent 1100 series instrument using a YMC-Pak ODS-A column of 4.6 mm i.d. for analysis and either 10 mm i.d. or 20 mm i.d. for purification. The purity of all test compounds was determined by HPLC analysis to be >90% pure.

N-((2S,3R)-3-hydroxy-4-((N-isobutyl-4-methoxyphenyl) sulfonamido)-1-phenylbutan-2-yl)-4-methyl-3-((4-(pyridin-3-yl)pyrimidin-2-yl)amino)benzamide (5a). To a stirring solution of commercially available carboxylic acid 10 (15 mg, 0.05 mmol) in DMF (1 mL) was added triethylamine (0.05 mL, 0.35 mmol), HATU (29 mg, 0.08 mmol), and amine 11 (0.06 mmol). The reaction was then stirred for 24 h at 23° C. The reaction mixture was concentrated under reduced pressure and purified via silica gel column chromatography (70% EtOAc/hexanes) to afford 5a (22 mg, 76%): ¹H NMR (500 MHz, CDCl₃) δ 9.30 (s, 1H), 8.73 (d, J=4.7 Hz, 1H), 8.62 (s, 1H), 8.51 (dd, J=5.2, 0.8 Hz, 1H), 8.35 (dt, J=7.9, 2.0 Hz, 1H), 7.72-7.66 (m, 2H), 7.45 (dd, J=8.0, 4.8 Hz, 1H), 7.32-7.10 (m, 7H), 6.95-6.89 (m, 2H), 6.54 (d, J=8.4 Hz, 1H), 4.42 (dt, J=7.4, 3.7 Hz, 1H), 4.08 (dt, J=8.0, 4.0 Hz, 1H), 3.82 (s, 3H), 3.22 (dd, J=15.0, 4.2 Hz, 1H), 3.14-3.00 (m, 3H), 2.94 (dd, J=13.5, 7.7 Hz, 1H), 2.84 (dd, J=13.4, 7.3 Hz, 1H), 2.38 (s, 3H), 1.90 (dq, J=13.7, 6.8 Hz, 1H), 0.85 (dd, J=11.7, 6.6 Hz, 6H); ¹³C NMR (125 MHz, CDCl₃) δ 168.10, 163.01, 162.64, 160.45, 159.31, 151.56, 148.44, 138.23, 137.76, 134.87, 133.12, 131.45, 130.88, 130.42, 129.61, 129.59, 128.67, 126.63, 124.05, 121.93, 119.21, 114.38, 108.69, 72.65, 58.68, 55.69, 54.55, 53.69, 35.02, 29.84, 27.25, 20.23, 20.19, 18.30; LRMS-ESI (m/z): [M+H] 695.3. HRMS (ESI) m/z: [M+H] calcd C₃₈H₄₂N₆O₅SH 695.30157; found 695.30138.

N-((2S,3R)-4-((4-amino-N-isobutylphenyl) sulfonamido)-3-hydroxy-1-phenylbutan-2-yl)-4-methyl-3-((4-(pyridin-3-yl)pyrimidin-2-yl)amino)benzamide (5b). Inhibitor 5b was synthesized according to the same procedure as inhibitor 5a utilizing carboxylic acid 10 (12 mg, 0.04 mmol), DMF (1 mL), DIPEA (0.04 mL, 0.24 mmol), HATU (20 mg, 0.05 mmol), and amine 12 (0.05 mmol). The crude product was purified via silica gel column chromatography (5% methanolic ammonia/CH₂Cl₂) to afford 5b (20 mg, 74%): ¹H NMR (500 MHZ, CDCl₃) δ 9.31 (s, 1H), 8.74 (d, J=4.8 Hz, 1H), 8.62 (s, 1H), 8.53 (d, J=5.1 Hz, 1H), 8.37 (dt, J=8.1, 2.0 Hz, 1H), 7.54 (d, J=8.4 Hz, 2H), 7.46 (dd, J=8.0, 4.8 Hz, 1H), 7.36-7.16 (m, 8H), 7.13 (s, 1H), 6.63 (d, J=8.4 Hz, 2H), 5.16 (d, J=9.0 Hz, 1H), 4.45 (td, J=7.3, 3.8 Hz, 1H), 4.18 (s, 2H), 4.10 (dt, J=8.2, 4.2 Hz, 1H), 3.30-3.04 (m, 4H), 2.88 (ddd, J=50.0, 13.4, 7.5 Hz, 2H), 2.40 (s, 3H), 1.95-1.89 (m, 1H), 0.88 (dd, J=10.3, 6.5 Hz, 6H); ¹³C NMR (125 MHz, CDCl₃) δ 168.07, 162.64, 160.49, 159.36, 151.64, 150.74, 148.51, 138.28, 137.75, 134.77, 133.12, 132.62, 131.59, 130.84, 129.61, 129.58, 128.62, 126.64, 126.57, 123.99, 121.91, 119.39, 114.17, 108.68, 72.74, 58.83, 54.49, 54.41, 53.79, 35.03, 27.28, 20.24, 18.28, 14.24; LRMS-ESI (m/z): [M+H] 680.3. HRMS (ESI) m/z: [M+H] calcd C₃₇H₄₁N₇O₄SH 680.30189; found 680.30245.

N-((2S,3R)-1-(3,5-difluorophenyl)-3-hydroxy-4-((N-isobutyl-4-methoxyphenyl) sulfonamido) butan-2-yl)-4-methyl-3-((4-(pyridin-3-yl)pyrimidin-2-yl)amino)benzamide (5c). Inhibitor 5c was synthesized according to the same procedure as inhibitor 5a utilizing carboxylic acid 10 (15 mg, 0.05 mmol), DMF (1 mL), DIPEA (0.05 mL, 0.29 mmol), HATU (25 mg, 0.06 mmol), and amine 13 (0.06 mmol). The crude product was purified via silica gel column chromatography (30% acetone/hexanes) to afford 5c (15 mg, 41%): ¹H NMR (500 MHZ, CDCl₃) δ 9.34 (d, J=2.3 Hz, 1H), 8.77-8.68 (m, 2H), 8.53 (d, J=5.1 Hz, 1H), 8.33 (dt, J=8.1, 1.9 Hz, 1H), 7.75-7.67 (m, 2H), 7.46 (dd, J=8.0, 4.8 Hz, 1H), 7.29 (dd, J=8.9, 7.1 Hz, 1H), 7.21 (dd, J=5.2, 1.9 Hz, 1H), 7.10 (s, 1H), 6.97-6.92 (m, 2H), 6.90-6.81 (m, 2H), 6.66-6.56 (m, 1H), 5.52 (s, 1H), 4.41 (dt, J=8.9, 4.7 Hz, 1H), 4.10 (s, 1H), 3.83 (s, 3H), 3.23-3.16 (m, 1H), 3.14-2.94 (m, 4H), 2.84 (dd, J=13.4, 7.1 Hz, 1H), 2.40 (s, 3H), 1.89 (dt, J=13.9, 6.9 Hz, 1H), 0.87 (dd, J=18.6, 6.5 Hz, 6H); ¹³C NMR (125 MHz, CDCl₃) δ 168.10, 164.12, 164.01, 163.11, 162.56, 160.40, 159.44, 151.64, 148.50, 142.63, 137.88, 134.77, 132.67, 131.02, 130.25, 129.59, 124.03, 121.89, 118.33, 114.44, 112.55, 112.36, 108.80, 102.14, 72.57, 58.86, 55.72, 53.99, 53.74, 34.72, 27.29, 20.22, 18.30; LRMS-ESI (m/z): [M+H] 731.2. HRMS (ESI) m/z: [M+H] calcd C₃₈H₄₀F₂N₆O₅SH 731.28272; found 731.28315.

N-((2S,3R)-4-((4-amino-N-isobutylphenyl) sulfonamido)-1-(3,5-difluorophenyl)-3-hydroxybutan-2-yl)-4-methyl-3-((4-(pyridin-3-yl)pyrimidin-2-yl)amino)benzamide (5d). Inhibitor 5d was synthesized according to the same procedure as inhibitor 5a utilizing carboxylic acid 10 (15 mg, 0.05 mmol), DMF (1 mL), triethylamine (0.05 mL, 0.29 mmol), HATU (24 mg, 0.06 mmol), and amine 14 (0.06 mmol). The crude product was purified via silica gel column chromatography (50% acetone/hexanes) to afford 5d (32 mg, 89%): ¹H NMR (500 MHz, CDCl₃) δ 9.32 (d, J=2.3 Hz, 1H), 8.74 (dd, J=4.8, 1.6 Hz, 1H), 8.67 (d, J=2.0 Hz, 1H), 8.52 (d, J=5.1 Hz, 1H), 8.33 (dt, J=8.1, 1.9 Hz, 1H), 7.57-7.51 (m, 2H), 7.45 (dd, J=8.0, 4.8 Hz, 1H), 7.29 (dd, J=10.4, 8.5 Hz, 1H), 7.21 (dd, J=5.2, 1.9 Hz, 1H), 7.10 (s, 1H), 6.87-6.83 (m, 2H), 6.66-6.57 (m, 3H), 5.37 (s, 1H), 4.41 (dd, J=7.3, 3.6 Hz, 1H), 4.13 (s, 2H), 4.08 (s, 1H), 3.18 (dd, J=15.0, 4.5 Hz, 1H), 3.14-2.88 (m, 4H), 2.80 (dd, J=13.4, 7.1 Hz, 1H), 2.39 (s, 3H), 1.94-1.82 (m, 1H), 0.87 (dd, J=16.1, 6.6 Hz, 6H); ¹³C NMR (125 MHZ, CDCl₃) δ 168.07, 162.61, 160.44, 159.41, 151.67, 150.76, 148.50, 142.60, 137.87, 134.77, 133.01, 132.66, 131.26, 131.00, 129.61, 126.61, 124.02, 121.90, 118.67, 114.23, 112.55, 112.36, 108.78, 102.10, 72.70, 59.02, 54.01, 53.82, 34.76, 29.84, 27.34, 20.24, 18.31; LRMS-ESI (m/z): [M+H] 716.3. HRMS (ESI) m/z: [M+H] calcd C₃₇H₃₉F₂N₇O₄SH 716.28306; found 716.28351.

N-((2S,3R)-3-hydroxy-4-((N-isobutyl-4-methoxyphenyl) sulfonamido)-1-phenylbutan-2-yl)-4-methyl-3-((4-(pyridin-3-yl) thiazol-2-yl)amino)benzamide (5e). To a stirring solution of 19a (16.5 mg, 0.05 mmol) in CH₂Cl₂ (1.0 mL) was added triethylamine (0.04 mL, 0.32 mmol) and HATU (26.0 mg, 0.07 mmol) at 23° C. To the reaction was then added 11 (0.058 mmol), and the reaction was stirred for 24 h. The reaction mixture was washed with 1 M citric acid and then successively with H₂O. The organic layer was then washed with brine, dried over Na₂SO₄, filtered and concentrated under reduced pressure. The crude product was purified via silica gel column chromatography (40-45% EtOAc/hexanes) to afford 5e (11 mg, 29.7%): ¹H NMR (500 MHZ, CDCl₃) δ 9.12 (d, J=1.8 Hz, 1H), 8.51 (d, J=5.9 Hz, 1H), 8.24 (s, 1H), 8.06 (d, J=8.0 Hz, 1H), 7.68 (d, J=8.8 Hz, 2H), 7.46 (bs, 1H), 7.33-7.23 (m, 6H), 7.16 (t, J=7.3 Hz, 1H), 6.95-6.87 (m, 3H), 6.72 (d, J=8.4 Hz, 1H), 4.46-4.38 (m, 1H), 4.1-4.06 (m, 1H), 3.83 (s, 3H), 3.22-3.04 (m, 4H), 2.95-2.78 (m, 2H), 2.34 (s, 3H), 1.93-1.81 (m, 1H), 0.85 (t, J=6.4 Hz, 6H); ¹³C NMR (125 MHZ, CDCl₃) δ 167.66, 165.70, 163.08, 148.60, 148.32, 147.55, 138.88, 138.16, 133.66, 133.45, 131.99, 131.37, 130.45, 130.22, 129.61, 129.58, 128.72, 126.69, 123.81, 122.70, 118.38, 114.42, 104.03, 72.89, 58.81, 55.72, 54.55, 53.78, 35.16, 27.30, 20.23, 18.01; LRMS-ESI (m/z): [M+H] 700.3. HRMS (ESI) m/z: [M+H] calcd C₃₇H₄₁N₅O₅S₂H 700.262739; found 700.26288.

N-((2S,3R)-3-hydroxy-4-((N-isobutyl-4-methoxyphenyl) sulfonamido)-1-phenylbutan-2-yl)-2-methyl-3-((4-(pyridin-3-yl) thiazol-2-yl)amino)benzamide (5f). Inhibitor 5f was synthesized according to the same procedure as inhibitor 5e utilizing 19b (14.0 mg, 0.05 mmol), CH₂Cl₂ (1.0 mL), triethylamine (0.04 mL, 0.27 mmol), HATU (22.0 mg, 0.06 mmol), and 11 (0.05 mmol). The crude product was purified via silica gel column chromatography (50-60% EtOAc/hexanes) to afford 5f (16.7 mg, 53.9%): ¹H NMR (500 MHz, CD₃OD) δ 9.01 (s, 1H), 8.44 (d, J=4.3 Hz, 1H), 8.26 (dt, J=8.0, 1.6 Hz, 1H), 7.88 (d, J=7.9 Hz, 1H), 7.78 (d, J=8.9 Hz, 2H), 7.45 (dd, J=8.0, 4.9 Hz, 1H), 7.33-7.23 (m, 4H), 7.24-7.16 (m, 3H), 7.06 (d, J=8.9 Hz, 2H), 6.86 (d, J=7.5 Hz, 1H), 4.34-4.29 (m, 1H), 3.94-3.86 (m, 1H), 3.84 (s, 3H), 3.60 (dd, J=14.9, 2.9 Hz, 1H), 3.14-3.04 (m, 2H), 2.93 (dd, J=13.6, 6.8 Hz, 1H), 2.82 (s, 1H), 2.65 (dd, J=13.9, 11.8 Hz, 1H), 2.08 (dt, J=14.2, 7.0 Hz, 1H), 1.90 (s, 3H), 0.92 (dd, J=30.3, 6.6 Hz, 6H); ¹³C NMR (125 MHz, CD₃OD) δ 172.75, 168.69, 164.55, 148.67, 148.64, 147.70, 141.13, 140.21, 139.89, 135.21, 132.73, 132.20, 130.64, 130.52, 129.38, 127.55, 127.36, 127.32, 125.28, 124.80, 123.94, 115.39, 105.60, 74.57, 58.93, 56.19, 55.63, 54.11, 36.88, 28.11, 20.56, 20.48, 14.72; LRMS-ESI (m/z): [M+H] 700.5. HRMS (ESI) m/z: [M+H] calcd C₃₇H₄₁N₅O₅S₂H 700.262739; found 700.26285.

N-((2S,3R)-3-hydroxy-4-((N-isobutyl-4-methoxyphenyl) sulfonamido)-1-phenylbutan-2-yl)-3-methyl-5-((4-(pyridin-3-yl) thiazol-2-yl)amino)benzamide (5g). Inhibitor 5g was synthesized according to the same procedure as inhibitor 5e utilizing 19c (23.0 mg, 0.07 mmol), CH₂Cl₂ (1.2 mL), triethylamine (0.06 mL, 0.1 mmol), HATU (36.0 mg, 0.1 mmol), and 11 (0.08 mmol). The crude product was purified via silica gel column chromatography (50-60% EtOAc/hexanes) to afford 5g (11.2 mg, 22%): ¹H NMR (500 MHz, CD₃OD) δ 9.09 (d, J=1.8 Hz, 1H), 8.44 (dd, J=4.9, 1.6 Hz, 1H), 8.34 (dt, J=8.0, 1.9 Hz, 1H), 8.18 (d, J=9.2 Hz, 1H), 7.91 (s, 1H), 7.67-7.59 (m, 3H), 7.46 (ddd, J=8.0, 4.9, 0.9 Hz, 1H), 7.31-7.26 (m, 3H), 7.21 (t, J=7.6 Hz, 2H), 7.14-7.10 (m, 2H), 6.89-6.83 (m, 2H), 4.22 (d, J=9.6 Hz, 1H), 3.99 (td, J=8.3, 2.7 Hz, 1H), 3.72 (s, 3H), 3.41 (dd, J=15.2, 2.7 Hz, 1H), 3.10 (dd, J=13.5, 8.7 Hz, 1H), 2.92 (dd, J=15.2, 8.7 Hz, 1H), 2.88-2.74 (m, 3H), 2.40 (s, 3H), 2.09-2.02 (m, 1H), 0.90 (dd, J=38.4, 6.6 Hz, 8H); ¹³C NMR (125 MHz, CD₃OD) δ 170.39, 165.74, 164.42, 148.84, 148.55, 147.67, 142.72, 140.37, 140.26, 136.85, 135.45, 132.72, 131.49, 130.61, 130.37, 129.29, 127.23, 125.40, 121.94, 121.85, 115.24, 114.79, 105.68, 74.78, 61.53, 59.28, 56.05, 54.59, 36.94, 28.02, 21.69, 20.50, 14.45; LRMS-ESI (m/z): [M+H] 700.5. HRMS (ESI) m/z: [M+H] calcd C₃₇H₄₁N₅O₅S₂H 700.262739; found 700.26397.

N-((2S,3R)-3-hydroxy-4-((N-isobutyl-4-methoxyphenyl) sulfonamido)-1-phenylbutan-2-yl)-2-methyl-5-((4-(pyridin-3-yl) thiazol-2-yl)amino)benzamide (5h). Inhibitor 5 h was synthesized according to the same procedure as inhibitor 5e utilizing 19d (33.1 mg, 0.11 mmol), CH₂Cl₂ (1.8 mL), triethylamine (0.09 mL, 0.64 mmol), HATU (52.0 mg, 0.14 mmol), and 11 (0.12 mmol). The crude product was purified via silica gel column chromatography (60% EtOAc/hexanes) to afford 5h (24.1 mg, 32%): ¹H NMR (500 MHZ, CDCl₃) δ 9.12 (s, 1H), 8.47 (s, 1H), 8.01 (d, J=7.8 Hz, 2H), 7.71 (d, J=8.9 Hz, 2H), 7.61 (s, 1H), 7.27 (dt, J=15.0, 7.4 Hz, 3H), 7.17 (dd, J=15.3, 8.1 Hz, 2H), 7.04 (d, J=8.3 Hz, 1H), 6.91 (d, J=8.9 Hz, 2H), 6.84 (s, 1H), 6.51 (d, J=9.0 Hz, 1H), 4.59-4.49 (m, 1H), 4.13 (td, J=7.4, 3.7 Hz, 1H), 3.80 (s, 3H), 3.29-3.10 (m, 3H), 3.02-2.90 (m, 3H), 2.03 (s, 3H), 1.92 (tt, J=14.9, 7.6 Hz, 1H), 0.89 (dd, J=13.4, 6.6 Hz, 6H). ¹³C NMR (125 MHz, CDCl₃) δ 169.94, 164.73, 163.05, 148.16, 147.88, 147.69, 138.20, 138.12, 136.87, 133.25, 131.83, 130.80, 130.48, 129.60, 128.66, 127.92, 126.64, 123.81, 119.87, 117.06, 114.43, 114.06, 103.61, 72.91, 58.61, 55.69, 53.72, 34.74, 29.84, 27.29, 20.25, 18.79; LRMS-ESI (m/z): [M+H] 700.5. HRMS (ESI) m/z: [M+H] calcd C₃₇H₄₁N₅O₅S₂H 700.262739; found 700.26323.

N-((2S,3R)-3-hydroxy-4-((N-isobutyl-4-methoxyphenyl) sulfonamido)-1-phenylbutan-2-yl)-2,4-dimethyl-3-((4-(pyridin-3-yl) thiazol-2-yl)amino)benzamide (5i). Inhibitor 5i was synthesized according to the same procedure as inhibitor 5e utilizing 19e (17.8 mg, 0.06 mmol), CH₂Cl₂ (0.9 mL), triethylamine (0.05 mL, 0.33 mmol), HATU (27.0 mg, 0.07 mmol), and 11 (0.06 mmol). The crude product was purified via silica gel column chromatography (2% MeOH/CH₂Cl₂) to afford 5i (7.5 mg, 19%): ¹H NMR (800 MHZ, CDCl₃) § 8.80 (s, 1H), 8.37 (s, 1H), 8.01 (d, J=7.8 Hz, 1H), 7.72 (d, J=8.9 Hz, 2H), 7.29 (m, 4H), 7.21 (t, J=7.1 Hz, 1H), 7.07 (d, J=7.8 Hz, 1H), 6.96 (dd, J=15.9, 8.3 Hz, 3H), 6.79 (d, J=20.7 Hz, 1H), 6.74 (s, 1H), 4.46-4.36 (m, 1H), 4.08-4.00 (m, 1H), 3.86 (s, 3H), 3.26-3.12 (m, 3H), 3.02 (dd, J=14.2, 10.3 Hz, 1H), 2.92 (ddd, J=38.8, 13.4, 7.5 Hz, 2H), 2.28 (s, 3H), 2.16 (s, 3H), 1.91 (dt, J=13.7, 6.9 Hz, 1H), 0.89 (dd, J=27.0, 6.6 Hz, 6H); ¹³C NMR (200 MHZ, CDCl₃) δ 170.60, 170.40, 163.21, 148.06, 146.48, 139.16, 138.13, 137.70, 136.74, 135.02, 133.98, 130.77, 130.04, 129.61, 129.57, 128.86, 128.78, 127.94, 126.80, 126.61, 123.87, 114.54, 114.08, 103.67, 73.27, 59.07, 55.78, 54.56, 53.86, 35.06, 27.48, 20.28, 20.11, 18.57, 15.04; LRMS-ESI (m/z): [M+H] 714.3. HRMS (ESI) m/z: [M+H] calcd C₃₈H₄₃N₅O₅S₂H 714.27839; found 714.27875.

N-((2S,3R)-3-hydroxy-4-((N-isobutyl-4-methoxyphenyl) sulfonamido)-1-phenylbutan-2-yl)-4-methyl-3-((4-methylthiazol-2-yl)amino)benzamide (5j). Inhibitor 5j was synthesized according to the same procedure as inhibitor 5e utilizing 21a (17.0 mg, 0.05 mmol), CH₂Cl₂ (0.9 mL), triethylamine (0.05 mL, 0.33 mmol), HATU (27.0 mg, 0.07 mmol), and 11 (0.06 mmol). The crude product was purified via silica gel column chromatography (35% EtOAc/hexanes) to afford 5j (13.2 mg, 38.8%): ¹H NMR (500 MHZ, CDCl₃) δ 7.72-7.69 (m, 2H), 7.33-7.17 (m, 5H), 7.11 (d, J=8.3 Hz, 1H), 7.01 (d, J=2.5 Hz, 1H), 6.99-6.94 (m, 2H), 6.19 (s, 1H), 6.06 (d, J=8.6 Hz, 1H), 4.41-4.35 (m, 1H), 4.20 (bs, 1H), 4.01-3.98 (m, 1H), 3.86 (s, 3H), 3.20-3.11 (m, 3H), 3.05-2.91 (m, 2H), 2.93-2.85 (m, 1H), 2.28 (s, 3H), 2.16 (s, 3H), 1.92-1.87 (m, 1H), 0.90 (dd, J=17.6, 6.6 Hz, 6H); ¹³C NMR (125 MHZ, CDCl₃) δ 170.08, 164.33, 163.21, 148.96, 138.33, 137.90, 136.77, 132.17, 130.51, 130.01, 129.61, 129.55, 128.84, 126.84, 120.00, 116.83, 114.53, 102.42, 73.08, 59.07, 55.78, 54.38, 53.85, 35.15, 27.48, 20.27, 20.09, 18.97, 17.46; LRMS-ESI (m/z): [M+H] 637.2. HRMS (ESI) m/z: [M+H] calcd C₃₃H₄₀N₄O₅S₂H 637.25184; found 637.25175. N-((2S,3R)-3-hydroxy-4-((N-isobutyl-4-methoxyphenyl) sulfonamido)-1-phenylbutan-2-yl)-2-methyl-3-((4-methylthiazol-2-yl)amino)benzamide (5k). Inhibitor 5k was synthesized according to the same procedure as inhibitor 5e utilizing 21b (20.1 mg, 0.08 mmol), CH₂Cl₂ (1.35 mL), triethylamine (0.07 mL, 0.49 mmol), HATU (40.0 mg, 0.11 mmol), and 11 (0.09 mmol). The crude product was purified via silica gel column chromatography (35% EtOAc/hexanes) to afford 5k (13.2 mg, 25.3%): ¹H NMR (500 MHZ, CDCl₃) δ 7.75-7.69 (m, 2H), 7.64 (d, J=8.1 Hz, 1H), 7.32-7.27 (m, 3H), 7.25-7.22 (m, 1H), 7.15 (t, J=7.9 Hz, 1H), 7.01-6.95 (m, 2H), 6.76 (dd, J=7.6, 1.2 Hz, 1H), 6.17 (d, J=1.3 Hz, 1H), 6.01 (d, J=8.7 Hz, 1H), 4.39 (dd, J=9.2, 4.8 Hz, 1H), 4.00 (dt, J=7.5, 4.5 Hz, 1H), 3.87 (d, J=1.6 Hz, 3H), 3.22-3.11 (m, 3H), 3.01-2.83 (m, 3H), 2.28 (d, J=1.1 Hz, 3H), 2.04 (s, 3H), 1.95-1.87 (m, 1H), 0.96-0.86 (m, 6H); ¹³C NMR (125 MHZ, CDCl₃) δ 170.37, 165.60, 163.24, 139.67, 138.16, 137.85, 129.97, 129.61, 129.50, 128.83, 127.03, 126.88, 126.79, 126.63, 122.29, 121.59, 114.55, 102.54, 73.14, 59.08, 55.78, 54.36, 53.85, 35.18, 27.48, 20.28, 20.08, 17.29, 14.09; LRMS-ESI (m/z): [M+H] 637.2. HRMS (ESI) m/z: [M+H] calcd C₃₃H₄₀N₄O₅S2H 637.25184; found 637.25154.

N-((2S,3R)-3-hydroxy-4-((N-isobutyl-4-methoxyphenyl) sulfonamido)-1-phenylbutan-2-yl)-3-methyl-5-((4-methylthiazol-2-yl)amino)benzamide (51). Inhibitor 51 was synthesized according to the same procedure as inhibitor 5e utilizing 21c (28.4 mg, 0.09 mmol), CH₂Cl₂ (1.5 mL), triethylamine (0.07 mL, 0.52 mmol), HATU (43.0 mg, 0.11 mmol), and 11 (0.10 mmol). The crude product was purified via silica gel column chromatography (35% EtOAc/hexanes) to afford 51 (10.2 mg, 18.5%): ¹H NMR (800 MHZ, CDCl₃) δ 7.68 (dq, J=9.0, 2.4, 1.6 Hz, 2H), 7.42 (s, 1H), 7.32-7.24 (m, 4H), 7.22-7.17 (m, 1H), 7.02 (s, 1H), 6.95-6.91 (m, 2H), 6.49 (d, J=8.3 Hz, 1H), 6.21 (s, 1H), 4.39 (dq, J=8.2, 3.0 Hz, 1H), 4.00 (dt, J=8.4, 4.3 Hz, 1H), 3.84 (s, 3H), 3.19 (dd, J=15.1, 4.1 Hz, 1H), 3.12-3.08 (m, 3H), 2.88 (d, J=7.5 Hz, 2H), 2.34 (s, 3H), 2.29 (s, 3H), 1.89-1.83 (m, 1H), 0.86 (dd, J=9.7, 6.6 Hz, 6H); ¹³C NMR (200 MHz, CDCl₃) δ 167.92, 164.05, 163.17, 147.93, 140.62, 140.18, 137.92, 135.50, 129.99, 129.59, 128.82, 126.82, 122.00, 121.67, 114.49, 113.83, 102.45, 72.94, 58.98, 55.75, 54.72, 53.68, 35.23, 27.37, 21.64, 20.24, 20.13, 17.14; LRMS-ESI (m/z): [M+H] 637.3. HRMS (ESI) m/z: [M+H] calcd C₃₃H₄₀N₄O₅S₂H 637.25184; found 637.25228.

N-((2S,3R)-3-hydroxy-4-((N-isobutyl-4-methoxyphenyl) sulfonamido)-1-phenylbutan-2-yl)-2-methyl-5-((4-methylthiazol-2-yl)amino)benzamide (5m). Inhibitor 5m was synthesized according to the same procedure as inhibitor 5e utilizing 21d (25.0 mg, 0.1 mmol), CH₂Cl₂ (0.17 mL), triethylamine (0.084 mL, 0.6 mmol), HATU (49.0 mg, 0.13 mmol), and 11 (0.11 mmol). The crude product was purified via silica gel column chromatography (35-40% EtOAc/hexanes) to afford 5m (13.4 mg, 25.8%): ¹H NMR (500 MHZ, CDCl₃) δ 7.74-7.66 (m, 2H), 7.33-7.17 (m, 5H), 7.11 (d, J=8.2 Hz, 1H), 7.01 (d, J=2.5 Hz, 1H), 6.99-6.93 (m, 2H), 6.19 (d, J=1.2 Hz, 1H), 6.07 (d, J=8.6 Hz, 1H), 4.37 (dq, J=9.4, 4.6 Hz, 1H), 3.99 (dd, J=7.4, 4.6 Hz, 1H), 3.86 (s, 3H), 3.22-3.09 (m, 3H), 3.06-2.82 (m, 3H), 2.28 (d, J=1.1 Hz, 3H), 2.16 (s, 3H), 1.90 (dt, J=13.8, 6.8 Hz, 1H), 0.90 (dd, J=17.8, 6.6 Hz, 6H); ¹³C NMR (125 MHZ, CDCl₃) δ 170.07, 164.43, 163.21, 138.27, 137.91, 136.78, 132.18, 130.58, 130.01, 129.61, 129.55, 128.83, 126.84, 120.02, 116.84, 114.53, 102.35, 73.08, 59.07, 55.78, 54.38, 53.85, 35.15, 29.85, 27.48, 20.27, 20.09, 18.97, 17.39; LRMS-ESI (m/z): [M+H] 637.3. HRMS (ESI) m/z: [M+H] calcd C₃₃H₄₀N₄O₅S₂H 637.25184; found 637.25186.

N-((2S,3R)-3-hydroxy-4-((N-isobutyl-4-methoxyphenyl) sulfonamido)-1-phenylbutan-2-yl)-4-methyl-3-((4-(trifluoromethyl) thiazol-2-yl)amino)benzamide (5n). Inhibitor 5n was synthesized according to the same procedure as inhibitor 5e utilizing 24a (24.9 mg, 0.079 mmol), CH₂Cl₂ (1.0 mL), triethylamine (0.066 mL, 0.472 mmol), HATU (39.0 mg, 0.102 mmol), and 11 (0.094 mmol). The crude product was purified via silica gel column chromatography (50% EtOAc/hexanes) to afford 5n (25.9 mg, 35%): 1H NMR (500 MHZ, CDCl₃) δ 7.89 (d, J=1.8 Hz, 1H), 7.79-7.69 (m, 3H), 7.32 (dd, J=9.9, 5.8 Hz, 7H), 7.12-6.96 (m, 3H), 6.57 (d, J=8.3 Hz, 1H), 4.43 (dd, J=7.9, 4.5 Hz, 1H), 4.09-4.03 (m, 1H), 3.90 (s, 3H), 3.30-3.10 (m, 4H), 2.92 (dd, J=7.6, 3.0 Hz, 2H), 2.37 (s, 3H), 1.96-1.87 (m, 1H), 0.90 (s, 7H). ¹³C NMR (126 MHz, CDCl₃) δ 167.96, 167.16, 163.07, 138.38, 137.82, 134.37, 133.41, 131.59, 129.90, 129.47, 129.43, 128.68, 126.68, 123.62, 121.39, 120.06, 119.24, 114.38, 109.74, 72.82, 58.82, 55.65, 54.67, 53.52, 45.95, 35.09, 30.98, 29.74, 27.25, 20.12, 20.01, 17.88, 8.64; LRMS-ESI (m/z): LRMS-ESI (m/z): [M+H] 691.2. HRMS (ESI) m/z: [M+H] calcd C₃₃H₃₇F₃N₄O₅S₂H 691.22357; found 691.22424.

N-((2S,3R)-3-hydroxy-4-((N-isobutyl-4-methoxyphenyl) sulfonamido)-1-phenylbutan-2-yl)-2-methyl-3-((4-(trifluoromethyl) thiazol-2-yl)amino)benzamide (50). Inhibitor 5o was synthesized according to the same procedure as inhibitor 5e utilizing 24b (20.1 mg, 0.063 mmol), CH₂Cl₂ (1.0 mL), triethylamine (0.05 mL, 0.378 mmol), HATU (31.1 mg, 0.08 mmol), and 11 (0.08 mmol). The crude product was purified via silica gel column chromatography (50% EtOAc/hexanes) to afford 50 (15.4 mg, 35.4%): 1H NMR (500 MHz, CDCl₃) δ 7.76-7.68 (m, 2H), 7.56-7.44 (m, 1H), 7.30 (d, J=6.3 Hz, 4H), 7.25-7.16 (m, 2H), 7.03-6.94 (m, 3H), 6.89 (dd, J=8.1, 2.0 Hz, 1H), 6.10 (dd, J=18.5, 8.8 Hz, 1H), 4.42 (s, 1H), 3.87 (s, 3H), 3.16 (ddd, J=14.6, 8.7, 5.8 Hz, 3H), 3.00-2.93 (m, 2H), 2.91-2.84 (m, 1H), 2.05 (d, J=10.2 Hz, 3H), 1.91 (t, J=7.0 Hz, 1H), 0.91 (dd, J=14.3, 6.5 Hz, 6H); ¹³C NMR (126 MHz, CDCl₃) δ 172.15, 171.31, 163.53, 137.95, 137.60, 136.98, 131.81, 131.54, 129.59, 129.33, 129.18, 129.02, 127.85, 127.27, 126.56, 126.29, 126.00, 114.70, 73.04, 58.97, 55.81, 54.60, 53.57, 53.11, 34.51, 31.09, 29.85, 27.44, 26.92, 20.15, 19.89, 19.46, 14.23. LRMS-ESI (m/z): [M+H] 691.2. HRMS (ESI) m/z: [M+H] calcd C₃₃H₃₇F₃N₄O₅S₂H 691.22357; found 691.22400.

N-((2S,3R)-3-hydroxy-4-((N-isobutyl-4-methoxyphenyl) sulfonamido)-1-phenylbutan-2-yl)-5-((4-(methoxymethyl) thiazol-2-yl)amino)-2-methylbenzamide (5p). Inhibitor 5p was synthesized according to the same procedure as inhibitor 5e utilizing 27 (17.4 mg, 0.06 mmol), CH₂Cl₂ (1.1 mL), triethylamine (0.05 mL, 0.38 mmol), HATU (31.0 mg, 0.08 mmol), and 11 (0.07 mmol). The crude product was purified via silica gel column chromatography (35% EtOAc/hexanes) to afford 5p (10.6 mg, 25.2%): ¹H NMR (800 MHZ, CDCl₃) δ 7.71 (d, J=8.8 Hz, 2H), 7.31-7.27 (m, 3H), 7.24-7.19 (m, 2H), 7.12 (d, J=8.1 Hz, 2H), 6.98-6.96 (m, 3H), 6.53 (s, 1H), 6.07 (d, J=8.7 Hz, 1H), 4.40 (s, 2H), 4.18 (s, 1H), 4.01 (d, J=6.1 Hz, 1H), 3.87 (s, 3H), 3.44 (s, 3H), 3.18-3.14 (m, 3H), 3.01 (dd, J=14.3, 10.0 Hz, 1H), 2.96-2.93 (m, 1H), 2.88 (d, J=6.4 Hz, 1H), 2.17 (s, 3H), 1.90 (dt, J=14.0, 6.8 Hz, 1H), 0.90 (dd, J=26.3, 6.6 Hz, 6H); ¹³C NMR (200 MHZ, CDCl₃) δ 169.96, 163.22, 138.07, 137.93, 136.87, 132.26, 130.04, 129.62, 129.55, 129.37, 128.85, 127.95, 126.85, 120.24, 117.16, 114.54, 114.08, 105.18, 73.09, 70.53, 59.06, 58.71, 55.79, 54.37, 53.83, 35.08, 27.48, 20.28, 20.10, 19.00; LRMS-ESI (m/z): [M+H] 667.2. HRMS (ESI) m/z: [M+H] calcd C₃₄H₄₂N₄O₆S₂H 667.26241; found 667.26216.

Methyl 3-(3-acetylthioureido)-4-methylbenzoate (16a). To a solution of potassium thiocyanate (292 mg, 3.0 mmol) in acetone (7.5 mL) was added acetyl chloride (0.14 mL, 2.0 mmol) dropwise. The reaction mixture was stirred at 60° C. for 1.5 h. To the reaction was then added methyl 3-amino-4-methylbenzoate (165 mg, 1.0 mmol) and the reaction was stirred at 23° C. for 4 h. The reaction was then filtered and concentrated under reduced pressure. The crude product was purified via silica gel column chromatography (25% ethyl acetate/hexanes) to afford the desired thiourea in quantitative yield as a white solid: ¹H NMR (500 MHz, CD₃OD) δ 8.27 (d, J=1.8 Hz, 1H), 7.85 (dd, J=8.0, 1.8 Hz, 1H), 7.39 (d, J=8.0 Hz, 1H), 3.90 (d, J=0.8 Hz, 3H), 2.33 (s, 3H), 2.19 (d, J=0.8 Hz, 3H); ¹³C NMR (125 MHZ, CDCl₃) δ 181.89, 173.93, 173.15, 168.03, 140.84, 138.52, 131.82, 129.59, 129.01, 52.64, 23.87, 18.30.

Methyl 3-(3-acetylthioureido)-2-methylbenzoate (16b). Thiourea 16b was synthesized according to the same procedure as thiourea 16a utilizing potassium thiocyanate (292 mg, 3.0 mmol), acetone (7.5 mL), acetyl chloride (0.14 mL, 2.0 mmol), methyl 3-amino-2-methylbenzoate (165 mg, 1.0 mmol). The crude mixture was purified via silica gel column chromatography (25% ethyl acetate/hexanes) to afford the desired thiourea in quantitative yield as a white solid: ¹H NMR (500 MHZ, CDCl₃) δ 9.39 (s, 1H), 7.85 (d, J=7.7 Hz, 1H), 7.69 (d, J=7.8 Hz, 1H), 7.31 (t, J=7.9 Hz, 1H), 3.90 (s, 3H), 2.50 (s, 3H), 2.22 (s, 3H); ¹³C NMR (125 MHZ, CDCl₃) δ 180.24, 171.67, 167.91, 137.46, 135.87, 131.83, 130.97, 130.17, 126.04, 52.29, 24.56, 15.37.

Methyl 3-(3-acetylthioureido)-5-methylbenzoate (16c). Thiourea 16c was synthesized according to the same procedure as thiourea 16a utilizing potassium thiocyanate (292 mg, 3.0 mmol), acetone (7.5 mL), acetyl chloride (0.14 mL, 2.0 mmol), methyl 3-amino-2-methylbenzoate (165 mg, 1.0 mmol). The crude mixture was purified via silica gel column chromatography (25% ethyl acetate/hexanes) to afford methyl 3-(3-acetylthioureido)-5-methylbenzoate (138 mg, 42.5%): ¹H NMR (500 MHZ, CDCl₃) § 12.31 (s, 1H), 8.90 (s, 1H), 3.91 (s, 3H), 2.42 (2, 3H), 2.22 (s, 3H); ¹³C NMR (125 MHZ, CDCl₃) δ 178.71, 171.20, 166.60, 139.36, 137.69, 131.01, 129.41, 128.89, 122.81, 52.42, 24.66, 21.44.

Methyl 5-(3-acetylthioureido)-2-methylbenzoate (16d). Thiourea 16d was synthesized according to the same procedure as thiourea 16a utilizing potassium thiocyanate (353 mg, 3.6 mmol), acetone (9 mL), acetyl chloride (0.17 mL, 2.4 mmol), 5-amino-2-methylbenzoate (200 mg, 1.2 mmol). The crude mixture was purified via silica gel column chromatography (25% ethyl acetate/hexanes) to afford methyl 5-(3-acetylthioureido)-2-methylbenzoate (181 mg, 56%): ¹H NMR (500 MHZ, CDCl₃) δ 8.87 (s, 1H), 8.08 (d, J=2.2 Hz, 1H), 7.76 (dd, J=8.3, 2.3 Hz, 1H), 7.28 (d, J=8.3 Hz, 1H), 3.90 (s, 3H), 2.60 (s, 3H), 2.22 (s, 3H); ¹³C NMR (125 MHZ, CDCl₃) δ 178.74, 171.18, 167.24, 139.40, 135.31, 132.35, 130.04, 128.05, 126.67, 52.18, 24.66, 21.57.

Methyl 3-(3-acetylthioureido)-2,4-dimethylbenzoate (16e). Thiourea 16e was synthesized according to the same procedure as thiourea 16a utilizing potassium thiocyanate (33 mg, 0.34 mmol), acetone (0.84 mL), acetyl chloride (0.02 mL, 0.22 mmol), and methyl 3-amino-2,4-dimethylbenzoate (0.02 mg, 0.11 mmol). The crude product was purified via silica gel column chromatography (30% ethyl acetate/hexanes) to afford methyl 3-(3-acetylthioureido)-2,4-methylbenzoate (26.7 mg, 85%): ¹H NMR (500 MHZ, CDCl₃) δ 9.91 (s, 1H), 7.82 (d, J=8.0 Hz, 1H), 7.17 (d, J=8.1 Hz, 1H), 3.88 (s, 3H), 2.48 (s, 3H), 2.31 (s, 3H), 2.22 (s, 3H); ¹³C NMR (125 MHZ, CDCl₃) δ 180.55, 172.07, 167.86, 140.28, 137.77, 136.42, 130.64, 129.18, 127.95, 52.09, 24.43, 18.90, 15.67; LRMS-ESI (m/z): [M+H]⁺ 281.0.

Methyl 4-methyl-3-((4-(pyridin-3-yl) thiazol-2-yl)amino)benzoate (18a). To a stirring solution of 16a (58 mg, 0.22 mmol) in dry methanol (1.3 mL) was added K₂CO₃ (182 mg, 1.32 mmol) and stirred for 5 minutes. To the reaction was then added 3-(bromoacetyl)pyridine·HBr (62 mg, 0.22 mmol) and the reaction was stirred for 6 h. The reaction mixture was then concentrated under reduced pressure and diluted with ethyl acetate and water. The aqueous layer was extracted with ethyl acetate (3×) and the combined organic layers were washed with brine, dried over Na₂SO₄, filtered, and concentrated under reduced pressure. The crude product was purified via silica gel column chromatography (50% ethyl acetate/hexanes) to afford 18a as a white solid (45 mg, 60%): ¹H NMR (500 MHz, CDCl₃) δ 9.14 (d, J=2.3 Hz, 1H), 8.55-8.47 (m, 2H), 8.11 (dt, J=7.9, 2.0 Hz, 1H), 7.76 (dd, J=7.9, 1.7 Hz, 1H), 7.59-7.51 (bs, 1H), 7.32-7.27 (m, 2H), 6.91 (s, 1H), 3.93 (s, 3H), 2.39 (s, 3H); ¹³C NMR (125 MHz, CDCl₃) δ 166.82, 166.18, 148.83, 148.60, 147.68, 138.90, 134.34, 133.43, 131.26, 130.53, 129.51, 125.63, 123.62, 121.52, 103.72, 52.36, 18.24; LRMS-ESI (m/z): [M+H]⁺ 326.1.

Methyl 4-methyl-3-((4-(pyridin-3-yl) thiazol-2-yl)amino)benzoate (18b). Thiazole 18b was synthesized according to the same procedure as thiazole 18a utilizing 16b (77.7 mg, 0.29 mmol), methanol (1 mL), K₂CO₃ (242 mg, 1.75 mmol), and 3-(bromoacetyl)pyridine·HBr (82 mg, 0.29 mmol). The crude product was then purified via silica gel column chromatography (50% ethyl acetate/hexanes) to afford 18b as a white solid (58 mg, 61%): ¹H NMR (500 MHz, CDCl₃) δ 9.29 (s, 1H), 8.64 (bs, 1H), 8.52 (d, J=4.0 Hz, 1H), 8.07 (dt, J=7.9, 1.7 Hz, 1H), 7.87 (d, J=7.4 Hz, 1H), 7.73 (d, J=7.7 Hz, 1H), 7.33 (t, J=7.9 Hz, 1H), 7.28 (dd, J=7.8, 4.8 Hz, 1H), 6.87 (s, 1H), 3.95 (s, 3H), 2.57 (s, 3H); ¹³C NMR (125 MHZ, CDCl₃) δ 168.27, 168.11, 148.53, 148.40, 147.64, 140.12, 133.29, 132.76, 132.29, 130.59, 127.34, 126.66, 126.62, 123.44, 103.29, 52.22, 15.01; LRMS-ESI (m/z): [M+H]⁺ 326.1.

Methyl 3-methyl-5-((4-(pyridin-3-yl) thiazol-2-yl)amino)benzoate (18c). Thiazole 18c was synthesized according to the same procedure as thiazole 18a utilizing 16c (57 mg, 0.215 mmol), methanol (0.77 mL), K₂CO₃ (178 mg, 1.29 mmol), and 3-(bromoacetyl)pyridine·HBr (60 mg, 0.215 mmol). The crude product was purified via silica gel column chromatography (50% EtOAc/hexanes) to afford 17c (60 mg, 86%): ¹H NMR (500 MHz, CDCl₃) δ 9.21-9.15 (m, 1H), 8.56 (d, J=4.9 Hz, 1H), 8.17 (dt, J=8.1, 1.9 Hz, 1H), 7.96 (s, 1H), 7.57 (s, 1H), 7.51 (s, 1H), 7.37 (dd, J=8.0, 4.8 Hz, 1H), 6.96 (s, 1H), 3.93 (s, 3H), 2.42 (s, 3H); ¹³C NMR (125 MHZ, CDCl₃) δ167.01, 164.66, 148.28, 147.27, 140.51, 139.89, 133.87, 131.38, 130.72, 124.95, 123.86, 123.13, 116.37, 113.27, 103.79, 52.44, 21.68; LRMS-ESI (m/z): [M+H]⁺ 326.1.

Methyl 2-methyl-5-((4-(pyridin-3-yl) thiazol-2-yl)amino)benzoate (18d). Thiazole 18d was synthesized according to the same procedure as thiazole 18a utilizing 16d (24 mg, 0.09 mmol), methanol (0.32 mL), K₂CO₃ (75 mg, 0.54 mmol), 3-(bromoacetyl)pyridine·HBr (25 mg, 0.09 mmol). The crude product was purified via silica gel column chromatography (50% ethyl acetate/hexanes) to afford 17d (65 mg, 66%): ¹H NMR (500 MHZ, CDCl₃) δ 9.14 (d, J=1.6 Hz, 1H), 8.56 (d, J=6.3 Hz, 1H), 8.21 (dt, J=7.9, 1.7 Hz, 1H), 8.06 (d, J=2.5 Hz, 1H), 7.56 (dd, J=8.2, 2.6 Hz, 1H), 7.41 (dd, J=7.8, 5.0 Hz, 1H), 7.26 (d, J=8.2 Hz, 3H), 6.95 (s, 1H), 3.93 (s, 3H), 2.58 (s, 3H); ¹³C NMR (125 MHZ, CDCl₃) δ 167.66, 165.14, 147.50, 146.52, 138.05, 135.32, 134.49, 132.96, 130.42, 128.73, 127.39, 124.10, 122.24, 120.78, 103.94, 52.21, 21.28; LRMS-ESI (m/z): [M+H]⁺ 326.2.

Methyl 2,4-dimethyl-3-((4-(pyridin-3-yl) thiazol-2-yl)amino)benzoate (18e). Thiazole 18e was synthesized according to the same procedure as thiazole 18a utilizing 16e (12.7 mg, 0.05 mmol), methanol (0.28 mL), K₂CO₃ (0.04 mg, 0.27 mmol), 3-(bromoacetyl)pyridine·HBr (0.01 mg, 0.05 mmol. The crude product was purified via silica gel column chromatography (50% ethyl acetate/hexanes) to afford 17e (12 mg, 78.4%): ¹H NMR (500 MHZ, CDCl₃) δ 9.07 (d, J=2.0 Hz, 1H), 8.50 (dd, J=4.8, 1.4 Hz, 1H), 8.05 (dt, J=7.9, 1.9 Hz, 1H), 7.82 (d, J=8.0 Hz, 1H), 7.22 (d, J=8.1 Hz, 1H), 6.77 (s, 1H), 3.90 (s, 3H), 2.58 (s, 3H), 2.38 (s, 3H); ¹³C NMR (125 MHZ, CDCl₃) δ 170.52, 167.98, 149.03, 148.44, 148.00, 147.03, 141.81, 139.24, 138.27, 133.86, 130.37, 130.02, 128.60, 123.76, 103.41, 52.20, 19.01, 15.76; LRMS-ESI (m/z): [M+H]⁺ 340.1.

Methyl 4-methyl-3-((4-methylthiazol-2-yl)amino)benzoate (20a). To a stirring solution of chloroacetone (0.1 mL, 1.2 mmol) and KSCN (175 mg, 1.8 mmol) at 23° C. in dry methanol (0.8 mL) was stirred for 1 h. To the reaction was then added methyl 3-amino-4methylbenzoate (200 mg, 1.2 mmol) and stirred at 60° C. for 5 h. The reaction was then cooled to 23° C., filtered and concentrated under reduced pressure. The crude product was purified via silica gel column chromatography (25% ethyl acetate/hexanes) to afford methyl 20a (55 mg, 17.5%): ¹H NMR (500 MHz, CDCl₃) § 8.24 (s, 1H), 7.74 (d, J=6.4 Hz, 1H), 7.30 (d, J=7.9 Hz, 1H), 6.20 (s, 1H), 3.91 (s, 3H), 2.37 (s, 3H), 2.31 (s, 3H); ¹³C NMR (125 MHz, CDCl₃) δ 166.61, 165.91, 146.99, 138.49, 134.55, 131.26, 129.45, 125.71, 120.77, 102.19, 52.24, 18.16, 16.76; LRMS-ESI (m/z): [M+H]⁺ 263.0.

Methyl 2-methyl-3-((4-methylthiazol-2-yl)amino)benzoate (20b). Thiazole 20b was synthesized according to the same procedure as thiazole 20a utilizing chloroacetone (0.1 mL, 1.2 mmol), KSCN (175 mg, 1.8 mmol), methanol (0.8 mL), and methyl 3-amino-2-methylbenzoate (200 mg, 1.2 mmol). The crude product was purified via silica gel column chromatography (25% EtOAc/hexanes) to afford methyl 20b (100 mg, 32%): ¹H NMR (500 MHZ, CDCl₃) δ 7.75 (dd, J=8.0, 1.8 Hz, 1H), 7.63 (dd, J=7.8, 1.4 Hz, 1H), 7.33-7.17 (m, 1H), 6.14 (dt, J=2.8, 1.1 Hz, 1H), 3.90 (d, J=0.9 Hz, 3H), 2.50 (s, 3H), 2.25 (dd, J=3.1, 1.2 Hz, 3H); ¹³C NMR (125 MHZ, CDCl₃) δ 168.45, 166.98, 148.90, 140.13, 132.37, 131.70, 126.81, 126.65, 125.38, 102.30, 52.28, 17.38, 14.89; LRMS-ESI (m/z): [M+H]⁺ 263.2.

Methyl 3-methyl-5-((4-methylthiazol-2-yl)amino)benzoate (20c). Thiazole 20c was synthesized according to the same procedure as thiazole 20a utilizing chloroacetone (0.05 mL, 0.61 mmol), KSCN (90 mg, 0.92 mmol), methanol (0.41 mL), methyl 3-amino-5-methylbenzoate (100 mg, 0.61 mmol). The crude product was purified via silica gel column chromatography (25% EtOAc/hexanes) to afford 20c (110 mg, 69%): ¹H NMR (500 MHZ, CDCl₃) § 7.80 (s, 1H), 7.53 (s, 1H), 7.40 (s, 1H), 6.19 (s, 1H), 3.90 (s, 3H), 2.37 (s, 3H), 2.30 (s, 3H); ¹³C NMR (125 MHZ, CDCl₃) δ 167.04, 165.14, 148.55, 141.04, 139.67, 131.22, 124.62, 123.37, 116.68, 102.11, 52.28, 21.50, 17.41; LRMS-ESI (m/z): [M+H] 263.1.

Methyl 2-methyl-5-((4-methylthiazol-2-yl)amino)benzoate (20d). Thiazole 20d was synthesized according to the same procedure as thiazole 20a utilizing chloroacetone (0.05 mL, 0.61 mmol), KSCN (90 mg, 0.92 mmol), methanol (0.41 mL), and methyl 3-amino-5-methylbenzoate (100 mg, 0.61 mmol). The crude product was purified via silica gel column chromatography (25% EtOAc/hexanes) to afford 20d (94 mg, 59%): ¹H NMR (500 MHZ, CDCl₃) δ 7.87 (d, J=2.6 Hz, 1H), 7.44 (dd, J=8.2, 2.6 Hz, 1H), 7.20 (d, J=8.3 Hz, 1H), 6.14 (s, 1H), 3.88 (s, 3H), 2.55 (s, 3H), 2.26 (s, 3H); ¹³C NMR (125 MHz, CDCl₃) δ 167.68, 166.06, 148.49, 138.75, 134.93, 132.77, 130.29, 122.78, 121.44, 101.60, 52.01, 21.17, 17.42; LRMS-ESI (m/z): [M+H] 263.1.

Methyl 4-methyl-3-((4-(trifluoromethyl) thiazol-2-yl)amino)benzoate (23a). Thiazole 23a was synthesized according to the same procedure as thiazole 18a utilizing 16a (150 mg, 0.636 mmol), methanol (3.25 mL), K₂CO₃ (528 mg, 3.816 mmol), 3-Bromo-1,1,1-trifluoroacetone (134 mg, 0.699 mmol). The crude product was purified via silica gel column chromatography (50% ethyl acetate/hexanes) to afford 23a (141 mg, 60% yield): ¹H NMR (500 MHZ, CDCl₃) δ 8.27 (s, 1H), 8.17 (d, J=1.7 Hz, 1H), 7.85 (dd, J=7.9, 1.7 Hz, 1H), 7.36 (d, J=7.9 Hz, 1H), 6.97 (s, 1H), 3.91 (s, 3H), 2.37 (s, 3H); ¹³C NMR (126 MHZ, CDCl₃) δ 169.53, 166.52, 141.01, 140.72, 138.41, 137.62, 131.62, 129.75, 127.51, 124.33, 121.42, 119.27, 109.39, 109.35, 52.38, 18.08. [M+H]⁺=317.2.

Methyl 2-methyl-3-((4-(trifluoromethyl) thiazol-2-yl)amino)benzoate (23b). Thiazole 23b was synthesized according to the same procedure as thiazole 18a utilizing 16b (90 mg, 0.338 mmol), methanol (1.20 mL), K₂CO₃ (280 mg, 2.025 mmol), 3-Bromo-1,1,1-trifluoroacetone (71 mg, 0.372 mmol). The crude product was purified via silica gel column chromatography (50% ethyl acetate/hexanes) to afford 23b (75.9 mg, 71% yield): ¹H NMR (400 MHZ, CDCl₃) δ 7.79 (dd, J=7.9, 1.3 Hz, 1H), 7.64 (dd, J=7.9, 1.4 Hz, 1H), 7.33 (td, J=7.9, 0.7 Hz, 1H), 6.96 (d, J=1.2 Hz, 1H), 3.93-3.90 (m, 3H), 2.53 (s, 3H); ¹³C NMR (101 MHZ, CDCl₃) δ 170.33, 167.98, 138.95, 134.64, 132.73, 129.18, 127.93, 127.06, 121.35, 118.67, 109.25, 52.39, 15.09. LRMS-ESI (m/z): [M+H]⁺ 317.1.

Methyl 5-((4-(methoxymethyl) thiazol-2-yl)amino)-2-methylbenzoate (26). Thiazole 26 was synthesized according to the same procedure as thiazole 18a utilizing 16d (12 mg, 0.05 mmol), methanol (0.45 mL), K₂CO₃ (6 mg mg, 0.05 mmol), and 1-bromo-3-methoxypropan-2-one (9 mg, 0.05 mmol). The crude product was purified via silica gel column chromatography (20% EtOAc/hexanes) to afford 26 (6 mg, 42%): ¹H NMR (500 MHZ, CDCl₃) δ 7.84 (d, J=2.6 Hz, 1H), 7.44 (dd, J=8.3, 2.6 Hz, 1H), 7.22 (d, J=8.3 Hz, 1H), 6.52 (s, 1H), 4.39 (s, 2H), 3.89 (s, 3H), 3.43 (s, 3H), 2.56 (s, 3H); ¹³C NMR (125 MHZ, CDCl₃) δ 167.66, 165.85, 149.51, 138.24, 135.28, 132.93, 130.46, 122.65, 121.04, 104.85, 70.51, 58.67, 52.14, 21.23; LRMS-ESI (m/z): [M+H]⁺ 293.1, [M+Na]⁺ 315.0.

4-Methyl-3-((4-(pyridin-3-yl) thiazol-2-yl)amino)benzoic acid (19a). To a stirring solution of 18a (0.028 g, 0.086 mmol) in 1 M LiOH (0.15 mL) was added a few drops of methanol and THF. The reaction was stirred until completion, and then the organic solvent was evaporated under reduced pressure. The aqueous layer was acidified to pH 3-5 utilizing 10% citric acid. The precipitate was collected via filtration and used without further purification.

2-Methyl-3-((4-(pyridin-3-yl) thiazol-2-yl)amino)benzoic acid (19b). Carboxylic acid 18b was synthesized according to the same procedure as carboxylic acid 19a utilizing 18b (53 mg, 0.16 mmol) and 1 M LiOH (0.5 mL).

3-Methyl-5-((4-(pyridin-3-yl) thiazol-2-yl)amino)benzoic acid (19c). Carboxylic acid 19c was synthesized according to the same procedure as carboxylic acid 19a utilizing 18c (0.02 g, 0.062 mmol) and 1 M LiOH (0.15 mL).

2-Methyl-5-((4-(pyridin-3-yl) thiazol-2-yl)amino)benzoic acid (19d). Carboxylic acid 19d was synthesized according to the same procedure as carboxylic acid 19a utilizing 18d (0.03 g, 0.09 mmol) and 1 M LiOH (0.28).

2,4-Dimethyl-3-((4-(pyridin-3-yl) thiazol-2-yl)amino)benzoic acid (19e). Carboxylic acid 19e was synthesized according to the same procedure as carboxylic acid 19a utilizing 18e (13 mg, 0.04 mmol) and 1 M LiOH (0.08). 4-Methyl-3-((4-methylthiazol-2-yl)amino)benzoic acid (21a).

Carboxylic acid 21a was synthesized according to the same procedure as carboxylic acid 19a utilizing 20a (0.05 mg, 0.18 mmol) and 1 M LiOH (0.55 mL).

3-Methyl-5-((4-methylthiazol-2-yl)amino)benzoic acid (21b). Carboxylic acid 21b was synthesized according to the same procedure as carboxylic acid 19a utilizing 20b (51.0 mg, 0.19 mmol) and 1 M LiOH (0.38 mL).

2-Methyl-3-((4-methylthiazol-2-yl)amino)benzoic acid (21c). Carboxylic acid 21c was synthesized according to the same procedure as carboxylic acid 19a utilizing 20c (19.4 mg, 0.074 mmol) and 1 M LiOH (0.15 mL).

2-Methyl-5-((4-methylthiazol-2-yl)amino)benzoic acid (21d). Carboxylic acid 21d was synthesized according to the same procedure as carboxylic acid 19a utilizing 20d (28.1 mg, 0.11 mmol) and 1 M LiOH (0.2 mL).

4-Methyl-3-((4-(trifluoromethyl) thiazol-2-yl)amino)benzoic acid (24a). Carboxylic acid 24a was synthesized according to the same procedure as carboxylic acid 19a utilizing 23a (0.280 g, 0.885 mmol) and 1 M LiOH (1.95 mL).

2-Methyl-3-((4-(trifluoromethyl) thiazol-2-yl)amino)benzoic acid (24b).

Carboxylic acid 24b was synthesized according to the same procedure as carboxylic acid 19a utilizing 23b (0.40 g, 0.127 mmol) and 1 M LiOH (1.55 mL).

5-((4-(Methoxymethyl) thiazol-2-yl)amino)-2-methylbenzoic acid (27).

Carboxylic acid 27 was synthesized according to the same procedure as carboxylic acid 19a utilizing 26 (22.0 mg, 0.08 mmol) and 1 M LiOH (0.23 mL).

1. A compound of the formula (I): 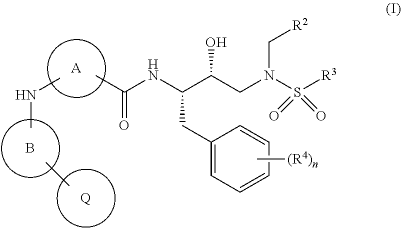
or a pharmaceutically acceptable salt thereof, wherein:
A is aryl;
B is heterocyclyl;
Q is alkyl or heterocyclyl;
R² is alkyl;
R³ is aryl, benzthiazole, benzoxazole, benzofuranyl or indolyl;
each R⁴ is, independently, alkyl, alkoxy, aryl, heterocyclyl, halo, hydroxy or amino; and
n is an integer from 0 to 3.

2. The compound of claim 1, or a pharmaceutically acceptable salt thereof, wherein A is a monocyclic aryl group, which is optionally substituted.

3.-5. (canceled)

6. The compound of claim 1, or a pharmaceutically acceptable salt thereof, wherein B has the formula: 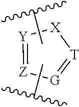
wherein X, Y, Z, G, and T are each, independently, C, CR⁵, N, NR⁶, S or O, wherein each R⁵ is independently H, alkyl or amino; and each R⁶ is independently H or alkyl.

7. The compound of claim 6, or a pharmaceutically acceptable salt thereof, wherein X is S and G is N.

8.-12. (canceled)

13. The compound of claim 6, or a pharmaceutically acceptable salt thereof, wherein B has the formula:

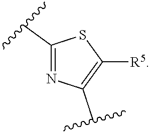

14. The compound of claim 6, wherein A, B, and Q are connected as shown:

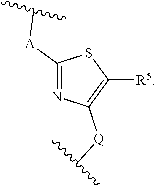

15. (canceled)

16. The compound of claim 1, or a pharmaceutically acceptable salt thereof, wherein B has the formula: 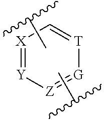
wherein X, Y, Z, G, and T are each, independently, C, CR⁵ or N, wherein each R⁵ is independently H, alkyl or amino.

17.-24. (canceled)

25. The compound of claim 16, or a pharmaceutically acceptable salt thereof, wherein B is of the formula:

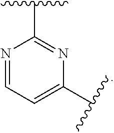 [SMILES: *c1ccnc(*)n1]

26. The compound of claim 16, or a pharmaceutically acceptable salt thereof, wherein A, B, and Q are connected as shown:

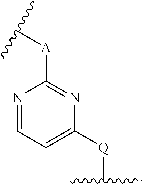

27. (canceled)

28. The compound of claim 1, or a pharmaceutically acceptable salt thereof, wherein Q has the formula: 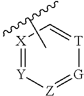
wherein X, Y, Z, G, and T are each, independently, C, CR⁵ or N, wherein each R⁵ is independently H, alkyl or amino.

29.-32. (canceled)

33. The compound of claim 1, or a pharmaceutically acceptable salt thereof, wherein Q is of the formula:

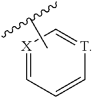

34.-36. (canceled)

37. The compound of claim 1, or a pharmaceutically acceptable salt thereof, wherein Q is of the formula:

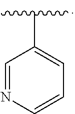 [SMILES: *c1cccnc1]

38. The compound of claim 1, or a pharmaceutically acceptable salt thereof, wherein Q is alkyl, which is optionally substituted.

39.-42. (canceled)

43. The compound of claim 38, or a pharmaceutically acceptable salt thereof, wherein Q is (C₁-C₃)alkyl-O—(C₁-C₃)alkyl.

44. (canceled)

45. The compound of claim 1, or a pharmaceutically acceptable salt thereof, wherein R³ is aryl.

46. The compound of claim 1, or a pharmaceutically acceptable salt thereof, wherein R³ is:

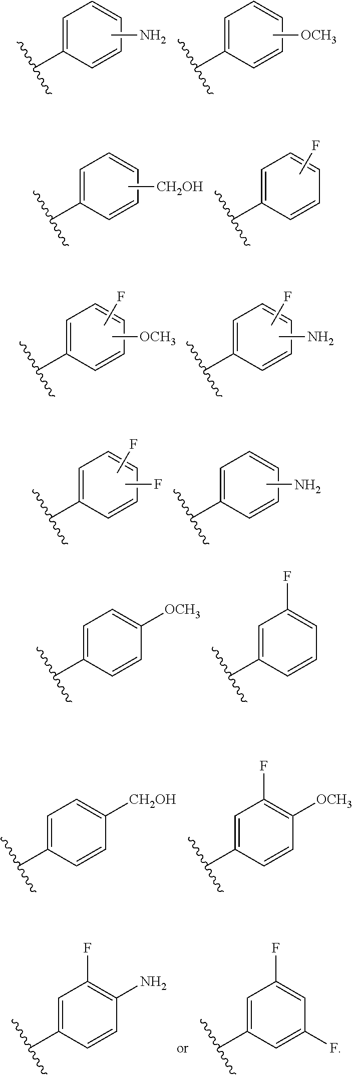

47. The compound of claim 1, or a pharmaceutically acceptable salt thereof, wherein R² is (C₁-C₆)alkyl.

48. (canceled)

49. (canceled)

50. The compound of claim 1, or a pharmaceutically acceptable salt thereof, wherein the compound has the formula:

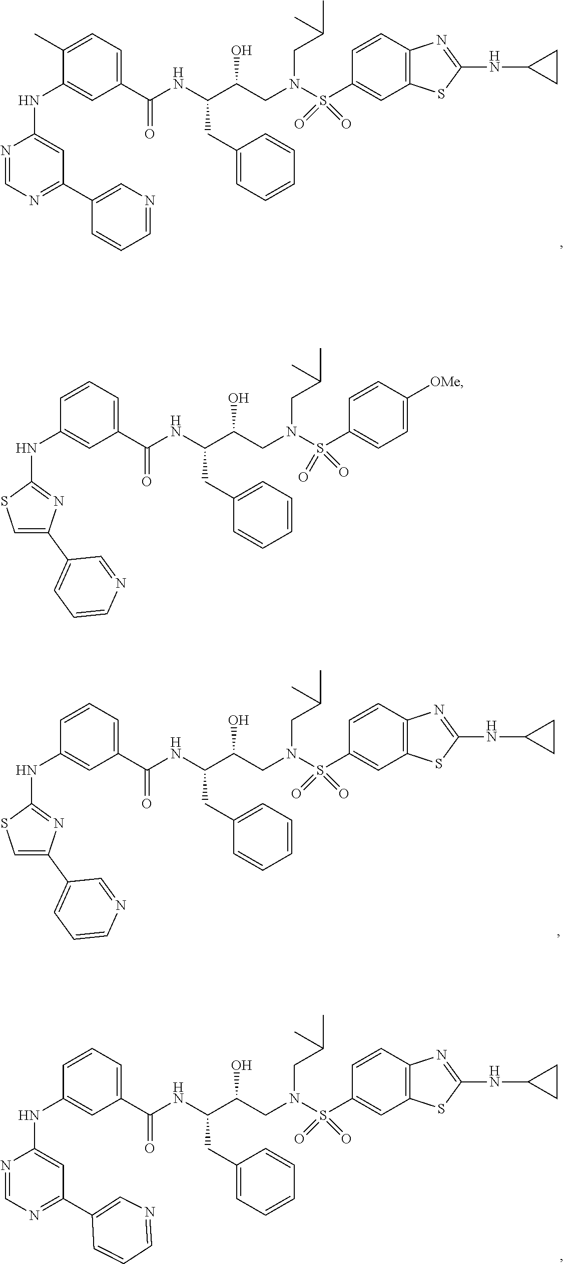 [SMILES: Cc1ccc(C(=O)N[C@@H](Cc2ccccc2)[C@H](O)CN(CC(C)C)S(=O)(=O)c2ccc3nc(NC4CC4)sc3c2)cc1Nc1cc(-c2cccnc2)ncn1 ; COc1ccc(S(=O)(=O)N(CC(C)C)C[C@@H](O)[C@H](Cc2ccccc2)NC(=O)c2cccc(Nc3nc(-c4cccnc4)cs3)c2)cc1 ; CC(C)CN(C[C@@H](O)[C@H](Cc1ccccc1)NC(=O)c1cccc(Nc2nc(-c3cccnc3)cs2)c1)S(=O)(=O)c1ccc2nc(NC3CC3)sc2c1 ; CC(C)CN(C[C@@H](O)[C@H](Cc1ccccc1)NC(=O)c1cccc(Nc2cc(-c3cccnc3)ncn2)c1)S(=O)(=O)c1ccc2nc(NC3CC3)sc2c1]

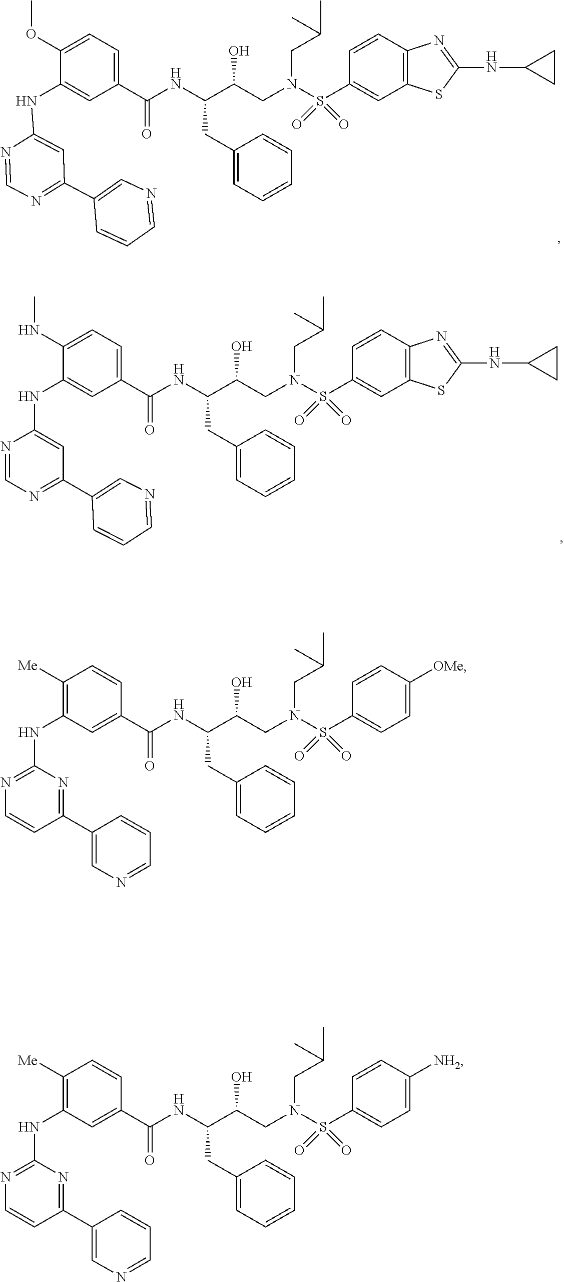 [SMILES: COc1ccc(C(=O)N[C@@H](Cc2ccccc2)[C@H](O)CN(CC(C)C)S(=O)(=O)c2ccc3nc(NC4CC4)sc3c2)cc1Nc1cc(-c2cccnc2)ncn1 ; CNc1ccc(C(=O)N[C@@H](Cc2ccccc2)[C@H](O)CN(CC(C)C)S(=O)(=O)c2ccc3nc(NC4CC4)sc3c2)cc1Nc1cc(-c2cccnc2)ncn1 ; COc1ccc(S(=O)(=O)N(CC(C)C)C[C@@H](O)[C@H](Cc2ccccc2)NC(=O)c2ccc(C)c(Nc3nccc(-c4cccnc4)n3)c2)cc1 ; Cc1ccc(C(=O)N[C@@H](Cc2ccccc2)[C@H](O)CN(CC(C)C)S(=O)(=O)c2ccc(N)cc2)cc1Nc1nccc(-c2cccnc2)n1]

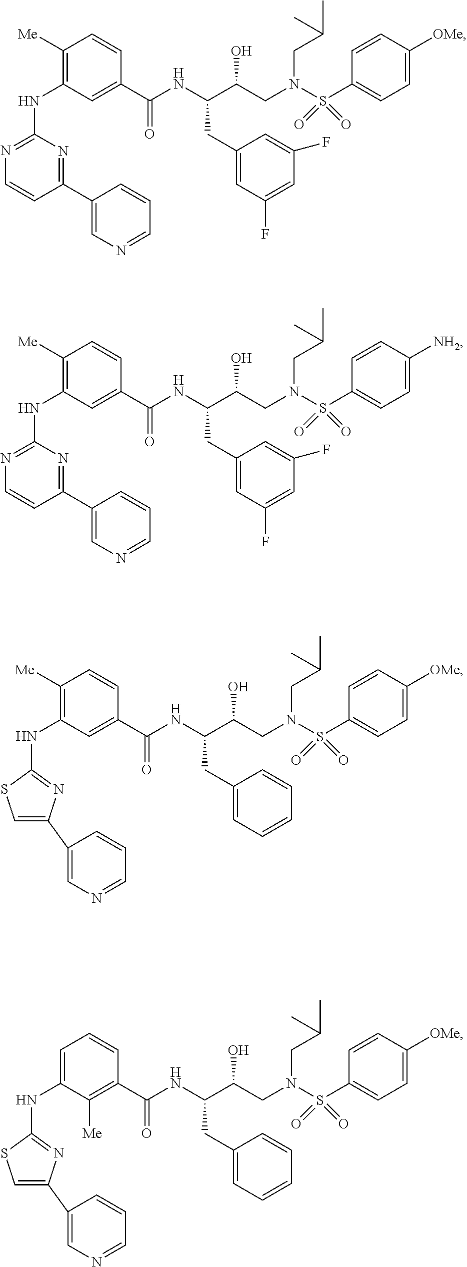 [SMILES: COc1ccc(S(=O)(=O)N(CC(C)C)C[C@@H](O)[C@H](Cc2cc(F)cc(F)c2)NC(=O)c2ccc(C)c(Nc3nccc(-c4cccnc4)n3)c2)cc1 ; Cc1ccc(C(=O)N[C@@H](Cc2cc(F)cc(F)c2)[C@H](O)CN(CC(C)C)S(=O)(=O)c2ccc(N)cc2)cc1Nc1nccc(-c2cccnc2)n1 ; COc1ccc(S(=O)(=O)N(CC(C)C)C[C@@H](O)[C@H](Cc2ccccc2)NC(=O)c2ccc(C)c(Nc3nc(-c4cccnc4)cs3)c2)cc1 ; COc1ccc(S(=O)(=O)N(CC(C)C)C[C@@H](O)[C@H](Cc2ccccc2)NC(=O)c2cccc(Nc3nc(-c4cccnc4)cs3)c2C)cc1]

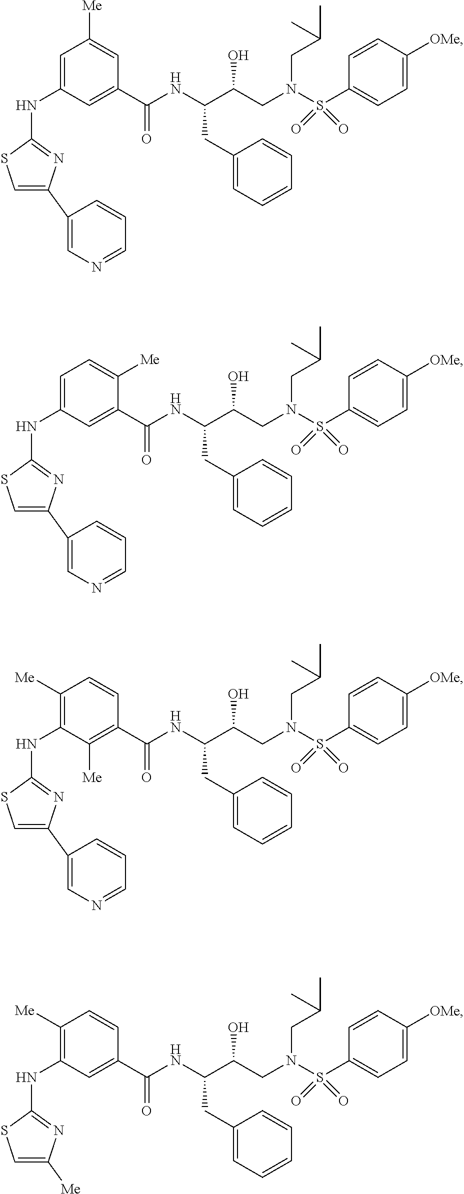 [SMILES: COc1ccc(S(=O)(=O)N(CC(C)C)C[C@@H](O)[C@H](Cc2ccccc2)NC(=O)c2cc(C)cc(Nc3nc(-c4cccnc4)cs3)c2)cc1 ; COc1ccc(S(=O)(=O)N(CC(C)C)C[C@@H](O)[C@H](Cc2ccccc2)NC(=O)c2cc(Nc3nc(-c4cccnc4)cs3)ccc2C)cc1 ; COc1ccc(S(=O)(=O)N(CC(C)C)C[C@@H](O)[C@H](Cc2ccccc2)NC(=O)c2ccc(C)c(Nc3nc(-c4cccnc4)cs3)c2C)cc1 ; COc1ccc(S(=O)(=O)N(CC(C)C)C[C@@H](O)[C@H](Cc2ccccc2)NC(=O)c2ccc(C)c(Nc3nc(C)cs3)c2)cc1]

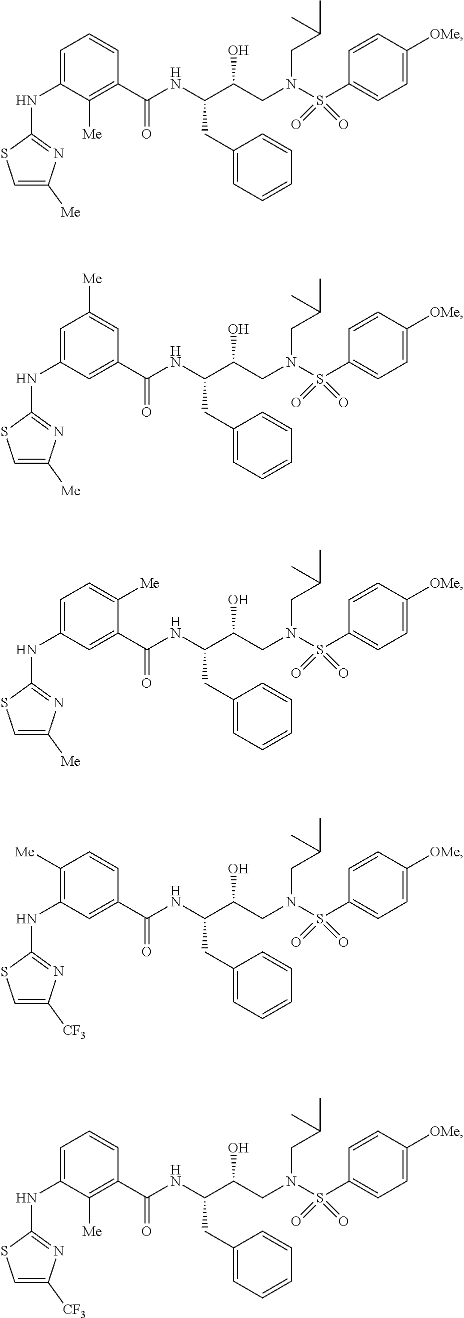 [SMILES: COc1ccc(S(=O)(=O)N(CC(C)C)C[C@@H](O)[C@H](Cc2ccccc2)NC(=O)c2cccc(Nc3nc(C)cs3)c2C)cc1 ; COc1ccc(S(=O)(=O)N(CC(C)C)C[C@@H](O)[C@H](Cc2ccccc2)NC(=O)c2cc(C)cc(Nc3nc(C)cs3)c2)cc1 ; COc1ccc(S(=O)(=O)N(CC(C)C)C[C@@H](O)[C@H](Cc2ccccc2)NC(=O)c2cc(Nc3nc(C)cs3)ccc2C)cc1 ; COc1ccc(S(=O)(=O)N(CC(C)C)C[C@@H](O)[C@H](Cc2ccccc2)NC(=O)c2ccc(C)c(Nc3nc(C(F)(F)F)cs3)c2)cc1 ; COc1ccc(S(=O)(=O)N(CC(C)C)C[C@@H](O)[C@H](Cc2ccccc2)NC(=O)c2cccc(Nc3nc(C(F)(F)F)cs3)c2C)cc1]

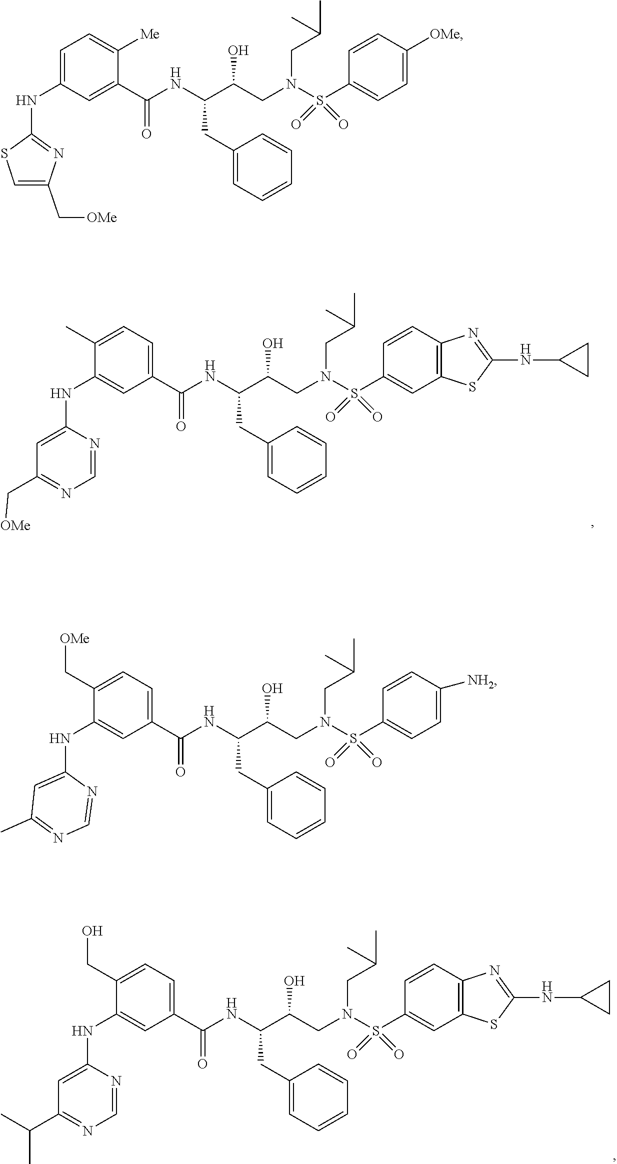 [SMILES: COCc1csc(Nc2ccc(C)c(C(=O)N[C@@H](Cc3ccccc3)[C@H](O)CN(CC(C)C)S(=O)(=O)c3ccc(OC)cc3)c2)n1 ; COCc1cc(Nc2cc(C(=O)N[C@@H](Cc3ccccc3)[C@H](O)CN(CC(C)C)S(=O)(=O)c3ccc4nc(NC5CC5)sc4c3)ccc2C)ncn1 ; COCc1ccc(C(=O)N[C@@H](Cc2ccccc2)[C@H](O)CN(CC(C)C)S(=O)(=O)c2ccc(N)cc2)cc1Nc1cc(C)ncn1 ; CC(C)CN(C[C@@H](O)[C@H](Cc1ccccc1)NC(=O)c1ccc(CO)c(Nc2cc(C(C)C)ncn2)c1)S(=O)(=O)c1ccc2nc(NC3CC3)sc2c1]

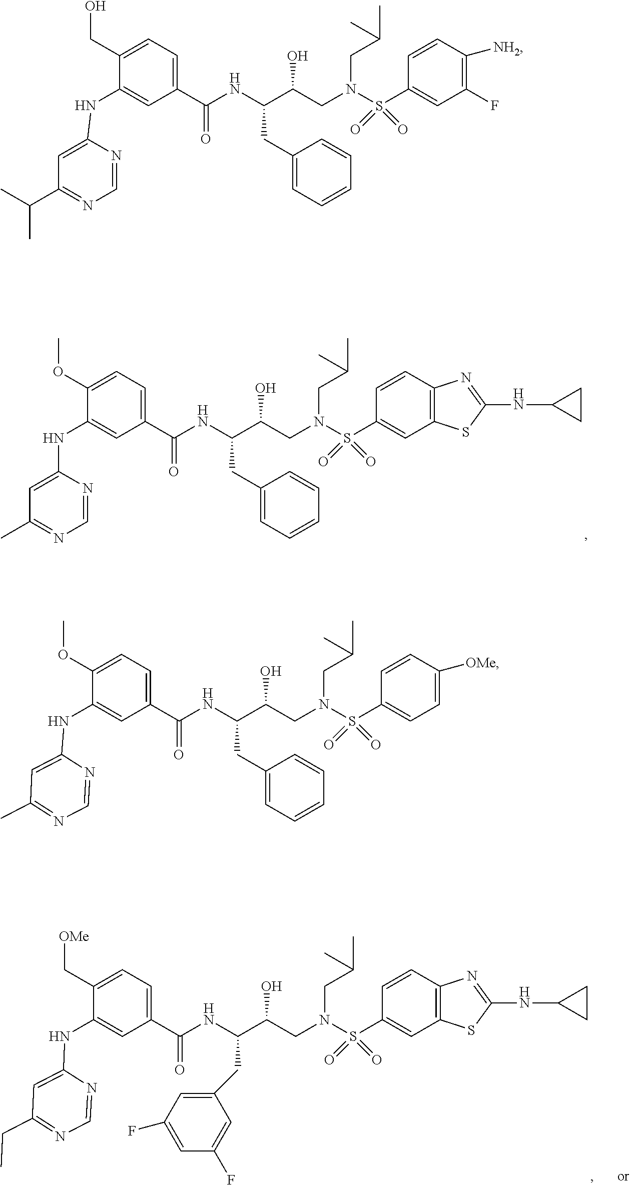 [SMILES: CC(C)CN(C[C@@H](O)[C@H](Cc1ccccc1)NC(=O)c1ccc(CO)c(Nc2cc(C(C)C)ncn2)c1)S(=O)(=O)c1ccc(N)c(F)c1 ; COc1ccc(C(=O)N[C@@H](Cc2ccccc2)[C@H](O)CN(CC(C)C)S(=O)(=O)c2ccc3nc(NC4CC4)sc3c2)cc1Nc1cc(C)ncn1 ; COc1ccc(S(=O)(=O)N(CC(C)C)C[C@@H](O)[C@H](Cc2ccccc2)NC(=O)c2ccc(OC)c(Nc3cc(C)ncn3)c2)cc1 ; CCc1cc(Nc2cc(C(=O)N[C@@H](Cc3cc(F)cc(F)c3)[C@H](O)CN(CC(C)C)S(=O)(=O)c3ccc4nc(NC5CC5)sc4c3)ccc2COC)ncn1]

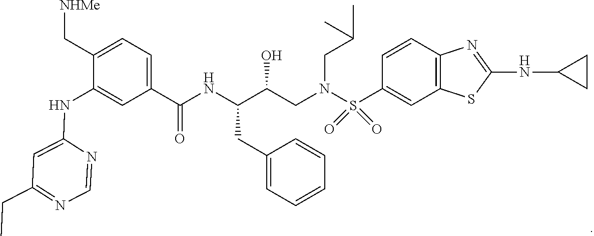 [SMILES: CCc1cc(Nc2cc(C(=O)N[C@@H](Cc3ccccc3)[C@H](O)CN(CC(C)C)S(=O)(=O)c3ccc4nc(NC5CC5)sc4c3)ccc2CNC)ncn1]

51. A pharmaceutical composition comprising the compound of claim 1, or a pharmaceutically acceptable salt or hydrate thereof, and one or more pharmaceutically acceptable excipients.

52. A method for treating an HIV infection or AIDS, the method comprising administering a therapeutically effective amount of one or more compounds of claim 1, or a pharmaceutical composition comprising same, or a pharmaceutically acceptable salt thereof, and one or more pharmaceutically acceptable excipients, to a subject in need thereof.

53.-57. (canceled)
